(19) United States
(12) Patent Application Publication
(10) Pub. No.: US 2023/0008870 A1
(43) Pub. Date: Jan. 12, 2023

(54) METHODS AND COMPOSITIONS FOR MONITORING AND DIAGNOSING HEALTHY AND DISEASE STATES

(71) Applicants: University of Utah Research Foundation, Salt Lake City, UT (US); Duke University, Durham, NC (US)

(72) Inventors: Jesse Worth Rowley, Salt Lake City, UT (US); Matthew Rondina, Salt Lake City, UT (US); Deepak Voora, Durham, NC (US)

(21) Appl. No.: 17/786,397

(22) PCT Filed: Dec. 18, 2020
(86) PCT No.: PCT/US20/66190

§ 371 Date: Jun. 16, 2022

Related U.S. Application Data
(60) Provisional application No. 62/951,004, filed on Dec. 20, 2019.

(51) Int. Cl. C12Q 1/6809; G01N 33/53; C12Q 1/6837; G16B 20/00; C12Q 1/6869

(52) U.S. Cl. CPC C12Q 1/6809; G01N 33/5308; C12Q 1/6837; G16B 20/00; C12Q 1/6869; G01N 2800/222; C12Q 2600/158; C12Q 2600/166

(57) ABSTRACT

Disclosed herein are methods for making a transcriptome-wide expression profile of a biological sample and identifying biomarkers that can be used to diagnose, monitor the onset, monitor the progression, and assess the recovery of a disease in a subject. The biomarkers can also be used to establish and evaluate treatment regimens.

### CROSS REFERENCE TO RELATED APPLICATIONS

This application claims the benefit of the filing date of U.S. Provisional Application 62/951,004, which was filed on Dec. 20, 2019. The content of this earlier filed application is hereby incorporated by reference herein in its entirety.

### STATEMENT REGARDING FEDERALLY FUNDED RESEARCH

This invention was made with government support under grant numbers HL144957 and AG048022 awarded by the National Institutes of Health. The government has certain rights in the invention.

### INCORPORATION OF THE SEQUENCE LISTING

The present application contains a sequence listing that is submitted via EFS-Web concurrent with the filing of this application, containing the file name “21101_0410P1_SL.txt” which is 4,096 bytes in size, created on Dec. 6, 2020, and is herein incorporated by reference in its entirety.

### FIELD OF THE INVENTION

The present disclosure relates to compositions and methods relating to a healthy gene expression signature reference for platelets that can be used for the monitoring and diagnosing of healthy and disease states in a subject. The healthy gene expression signature reference for platelets can be used to screen, diagnose, monitor the onset, monitor the progression, and identify and diagnose disease states or can be used as a prognostic indicator. The healthy gene expression signature reference for platelets can also be used to establish and evaluate treatment plans.

### BACKGROUND

Current gene expression diagnostics rely on using large cross-sectional cohorts that include healthy controls to account for noise from between and within-individual variation. No reference is available for expected variation in platelets from healthy individuals over time. Cross-sectional studies require large numbers of healthy controls to indirectly account for within-individual variation. These cross sectional studies do not directly correct for within individual variation or for genetic variants that can significantly influence gene expression. More precise “healthy” references are needed for diagnostic testing.

### BRIEF DESCRIPTION OF THE DRAWINGS

FIGS. 1A-F show within and between individual stability of platelet RNA expression over 4 months (cohort 1) and 4 years (cohort 2). FIGS. 1A and 1D show unsupervised clustering and heatmaps of total RNA expression in platelets from the samples in (FIG. 1A cohort 1 and (FIG. 1D) cohort 2. The histograms to the left of each heatmap show the distribution of distances between the pairs of samples, and the darkness of blue indicates the degree of similarity between pairs of samples. Samples that cluster as neighbors in the heatmap dendrograms reflect transcriptomes with the highest similarity. Nearest neighbor self-pairs are highlighted in yellow and gray, whereas nearest neighbor non-self-pairs are highlighted in orange. FIGS. 1B and 1E show an example individual correlation plots of the transcripts in (FIG. 1B) cohort 1 or (FIG. 1E) cohort 2. Each data point represents the regularized, log-transformed expression level (RLD) of a single transcript from the specified donor at time 0 (x axis) versus 0, 2 wk, 4 months, or 4 years (y axis) within the same individual (top panels) or a different individual (bottom panels). Points are heat-colored according to density. P values are from Pearson correlation. FIGS. 1C and 1F shows boxplots summarizing the RNA expression Pearson correlation between within versus between-individual pairs at (FIG. 1C) time 0 and 4 months or (FIG. 1F) in aggregate at all-time points tested (left) or at the individually specified time points (right). With regards to specified time points in FIG. 1F, note that the average within-individual correlation did not significantly decrease as samples taken farther apart were compared. For example, there was not a significant difference when comparing the average within-individual correlation of TO versus 2 weeks with the average within-individual correlation of TO versus 4 years. Boxplots for cohort 1 (FIG. 1C) are shown before and after adjusting for age, sex, and race, whereas they are not adjusted for cohort 2 (FIG. 1F), because of the smaller sample size. P values are from Wilcox test, adjusted.

FIGS. 2A-D show the comparison between cohorts of the within and total variation of each transcript in platelets. The mean within and total individual variation (standard deviation, SD) was calculated from the regularized log transformed expression (RLD) for each transcript. FIGS. 2A and 2C show that (FIG. 2A) within or (FIG. 2C) total individual variation of each transcript in cohort 1 (x-axis) plotted against the respective variation of each transcript in cohort 2 (y-axis). The horizontal and vertical lines at 0.5 marks an arbitrary threshold of variation used for the Venn diagrams in FIG. 2B and FIG. 2D. FIGS. 2B and 2D show Venn diagrams of the overlap in the transcripts with highest B) within and D) total variation. Listed below each Venn are the significantly enriched GO terms for the transcripts overlapping both cohorts. FDR=Benjamin False Discovery Rate calculated by David (Huang D W, et al. Nat Protoc. 2009; 4:44-57).

FIGS. 3A-F show the transcripts ranked by repeatability are enriched in heritable traits and eQTLs. FIG. 3A shows a table of transcripts with the highest repeatability in cohort 1 RNA-seq data, and their reported association with race, sex, or eQTLs in PRAX1 (Simon L M, et al. Am J Hum Genet. 2016; 98:883-97; and Simon L M, et al. Blood. 2014; 123(16):e37-45) microarray data. Associations with FDR<1e-4 are highlighted in pink. NS=not significant. FIG. 3B shows a correlation plot of RNA expression (log normalized) of MFN2 at time 0 (x-axis) and 4 months (y-axis). Points are colored according to rs1474868 genotype (ND=not determined). Above is a density histogram showing a bimodal distribution according to genotype. Bimodal P value from Hartigan's diptest for multi-modality. FIG. 3C shows, top: enrichment plots for the presence of eQTLs ranked according to different measures: within variation, mean expression abundance, total variation, or repeatability. The axis below the plot indicates the gene rank according to each measure, and indicates the value of the repeatability measure (the values of the other measures are not noted on the axis). Genes with a known eQTL are in red, those without are in blue. Thus, genes with the highest repeatability are nearly 100% eQTL genes, whereas those with the lowest repeatability are nearly 0%. FIG. 3C shows, bottom: plot of cumulative enrichment scores for each metric. FIG. 3D shows the odds ratios for the likelihood of identifying an eQTL for genes at the indicated repeatability thresholds compared to the same number of genes ranked by total variation. FIG. 3E shows boxplots of LINC01089 expression according to rs1168863 genotype in cohort 1 at time 0 and 4 months and in the NL cohort (Best M G, et al. Cancer Cell. 2017; 32:238-252). *P values adjusted for age, sex, and cohort 1) race or cohort 2) population structure (inferred genetic ancestry (Purcell S, et al. Am J Hum Genet. 2007; 81:559-75; and Chang C C, et al. Gigascience. 2015; 4:7)). FIG. 3F shows boxplots demonstrating allelic imbalance of rs1168863 within heterozygotes in cohort 1 and the NL cohort. The proportion of RNA-seq reads with A nucleotide versus T nucleotide was calculated and plotted for each heterozygote individual.

FIGS. 4A-C show within and between individual stability of exon skipping in platelets. FIG. 4A shows a schematic of how exon skipping events are defined. Percent exon Spliced In (PSI) is calculated using splice junction reads and is the ratio of exon inclusion junction reads over total junction reads. FIG. 4B shows correlation plots the PSI for the exon skipping events within (left panel) and between (right panel) individuals. Each point represents a single exon skipping event from the specified donor at time 0 (x axis) versus 0 or 4 months (y axis). FIG. 4C shows boxplots summarizing Pearson correlations of within versus between-individual pairs when analyzing PSI of all exon skipping events at time 0 and 4 months, and after adjusting for age, sex, and race. *Wilcox test, adjusted.

FIGS. 5A-D show repeatability of Exon 14 skipping in SELP and association with race. FIG. 5A shows a table of the most repeatable exon skipping events in platelets. FIG. 5B shows representative IGV plots of sequencing reads from two different individuals at time 0 and 4 months, showing the differential distribution of reads between individuals that align to or skip exon 14 of SELP. The histograms indicate the cumulative abundance of reads that aligned to each exon. A subset of individual reads is shown below each histogram that indicate split splice junction reads by thin lines (absent in read) that connect to thick lines (mapped portion of read). Red and blue reads are splice junction reads that align to or skip exon 14 respectively. FIG. 5C shows a correlation plot of SELP exon 14 PSI. Each point represents the PSI for an individual donor at time 0 (x-axis) and 4 months (y-axis). Donors represented in the IGV plots in B are labeled in red text. FIG. 5D shows a boxplot of SELP exon 14 mean PSI according to race at T=0 and T=4 months.

FIGS. 6A-C show rs6128 is a platelet SELP exon 14 splice QTL. FIG. 6A shows a close up IGV plot showing read distribution across SELP exon 14 for top) an individual with rs6128 A/A and relatively high levels of exon skipping reads or bottom) an individual with rs6128 (T/T) variant. The C->T change does not change amino acid sequence, but alters exonic splicing silencer and enhancer sites as predicted by Ex-Skip (Raponi M, et al. Hum Mutat. 2011; 32:436-444). FIG. 6B shows a boxplot of SELP exon 14 mean PSI according to rs6128 genotype inferred from RNA-seq in cohort 1 at time 0 and 4 months. FIG. 6C shows a boxplot of SELP exon 14 mean PSI according to rs6128 genotype inferred from RNA-seq data published in the NL cohort. *P values adjusted for age, sex, and FIG. 6B) race or FIG. 6C) population structure (inferred genetic ancestry (Purcell S, et al. Am J Hum Genet. 2007; 81:559-75; and Chang C C, et al. Gigascience. 2015; 4:7)).

FIGS. 7A-D show rs6128 directly regulates exon 14 skipping in SELP and alters the ratio of surface to soluble P-selectin protein expression. FIG. 7A is a schematic of mini-gene constructs of SELP that include the ORF of SELP, and the introns flanking exon 14. The C/C and T/T constructs vary by a single nucleotide at rs6128. Constructs were cloned into vectors with 2 different promoters (CMV or MSCV). After transfection into HEK 293 cells, the introns are spliced out and exon 14 is variably spliced out (skipped). The extent of exon 14 skipping is measured by PCR via exon 14 flanking primers that generate two PCR products of different sizes. FIG. 7B shows RT-PCR analysis of SELP exon 14 skipping following transfection of HEK 293 cells with rs6128 C/C or T/T vectors. Shown is a representative result from 5 independent experiments. Below are bar graphs and standard error summary of PSI calculated according to densitometry analysis of the exon 14 inclusion band (upper band) divided by the sum of the upper and lower bands (total). *paired t-test, n=5 independent experiments. FIG. 7C shows the flow cytometry analysis of P-selectin surface expression following transfection of HEK 293 cells with rs6128 C/C or T/T vectors. Top is a representative histogram overlay of P-selectin surface expression 24 hours after transfection with CMV promoter empty vector, rs6128 C/C, or T/T. Below are bar graph and standard error summaries of the fold change (normalized to transfection) of surface P-selectin MFI following transfection. *paired T test, n=5-6 pairs per group. FIG. 7D shows ELISA analysis of soluble P-selectin in supernatants of HEK 293 cells following transfection with rs6128 C/C or T/T vectors. *paired T test, n=12-14 pairs per group.

FIGS. 8A-F show within and between individual stability of platelet non-coding RNA expression over 4 months (cohort 1) and 4 years (cohort 2). FIGS. 8A and 8D show unsupervised clustering and heatmaps of non-coding RNA expression in platelets from the samples in (FIG. 8A) cohort 1 and (FIG. 8D) cohort 2. The histograms to the left of each heatmap show the distribution of distances between the pairs of samples, and the darkness of blue indicates the degree of similarity between pairs of samples. Samples that cluster as neighbors in the heatmap dendrograms reflect non-coding transcriptomes with the highest similarity. Nearest neighbor self-pairs are highlighted in yellow and gray, whereas nearest neighbor non-self-pairs are highlighted in orange. FIGS. 8B and 8E show example individual correlation plots of non-coding transcripts in (FIG. 8B) cohort 1 or (FIG. 8E) cohort 2. Each data point represents the regularized, log-transformed expression level (RLD) of a single non-coding transcript from the specified donor at time 0 (x axis) versus 0, 2 wk, 4 months, or 4 years (y axis) within the same individual (top panels) or a different individual (bottom panels). Points are heat-colored according to density. FIGS. 8C and 8F show boxplots summarizing the non-coding RNA expression Pearson correlation between all within versus between-individual pairs at (FIG. 8C) time 0 and 4 months or (FIG. 8F) in aggregate at time points (left) or at the individually specified time points (right). In FIG. 8C, boxplots for cohort 1 are shown before and after adjusting for age, sex, and race, whereas unadjusted are shown for cohort 2 (because of smaller sample size). P values from Wilcox test, adjusted.

FIGS. 9A-F show a comparison of the within-individual and total variation of each transcript in platelets. The mean within and total individual variation (standard deviation, SD) was calculated from the regularized log transformed expression (RLD) for each transcript. FIGS. 9A-B show normalized expression (x-axis) plotted against within individual variation for each transcript (y-axis) for (FIG. 9A) cohort 1 and (FIG. 9B) cohort 2. FIGS. 9C-D show normalized expression (x-axis) plotted against total variation for each transcript (y-axis) for (FIG. 9C) cohort 1 and (FIG. 9D) cohort 2. FIGS. 9E-F show total individual variation of each transcript (x-axis) plotted against the within-individual variation (y-axis) of each transcript for (FIG. 9E) cohort 1 and (FIG. 9F) cohort 2. Labeled points are representative transcripts with low-within and high total individual variation.

FIG. 10 shows variance partition analysis of platelet gene expression. Violin plots showing the distribution of the percent of variance for each transcript (y-axis) attributable to the indicated covariates (x-axis). The width of the violin indicates the probability density of transcripts at each y-value. Boxplots indicate median and interquartile range, and outliers are plotted as individual points. For example, sex explains less than 50% of the variation for most transcripts, except for the Y chromosome genes EIF1AY, TMSB4Y, and UTY which vary almost exclusively according to sex. The plot on the far right indicates that for most transcripts (>50%), differences between individuals explain the majority of variation.

FIGS. 11A-D show tables of transcripts with (FIG. 11A) the highest expression, (FIG. 11B) lowest within-individual variation, (FIG. 11C) highest total variation, or (FIG. 11D) highest repeatability (low within, high between individual variation) in cohort 1 RNA-seq data, and their reported association with race, sex, or eQTLs in PRAX1 microarray data. Associations with FDR<1e-4 are highlighted in pink. NS=not significant.

FIG. 12 shows that for transcripts with a reported eQTL in PRAX1, the FDR is associated with repeatability. On the x-axis is the lowest reported log FDR (i.e. −125=10⁻¹²⁵) for eQTLs associated with each transcript. On the y-axis is 1-repeatability (cohort 1) for each transcript.

FIG. 13 shows unsupervised clustering and heatmap based on the Exon PSI for all 245 identified exon skipping events in platelets within the 31 individuals in cohort 1 at T=0 and T=4 months. The histograms to the left show the distribution of distances between all pairs of samples, and the darkness of blue indicates the degree of similarity between pairs of samples. Samples that cluster as neighbors in the heatmap dendrograms reflect samples with the highest similarity in exon skipping levels. Nearest neighbor self-pairs are highlighted in yellow. Bars to the left are colored according to sequencing batch or lane.

FIG. 14 shows PCR confirmation of SELP exon 14 skipping in platelets. Platelet RNA from 5 different individuals was reverse transcribed and cDNA amplified with primers flanking exon 14 of SELP. Bands were extracted and sequenced by Sanger sequencing to confirm sequence.

FIGS. 15A-B show exon 14 skipping of SELP remains associated with rs6128 in disease. Boxplots of SELP exon 14 mean PSI according to rs6128 genotype inferred from RNA-seq in healthy and disease samples reported in the NL cohort when analyzed in (FIG. 15A) aggregate or (FIG. 15B) according to disease. In FIG. 15B, diseases with multiple samples of at least 2 different genotypes are shown. *p adjusted for age, sex, smoking, hospital, and storage time.

FIG. 16 shows that rs6128 directly regulates exon 14 skipping in P-selectin protein. Western blot analysis of P-selectin (antibody to c-terminal DYK tag) in HEK 293 cells following transfection of rs6128 C/C or T/T SELP (CMV promoter) constructs. Shown is a representative blot from 4 independent experiments. Below are bar graphs and standard error summary of PSI calculated according to densitometry analysis of the exon 14 inclusion band (upper band) divided by the sum of the upper and lower bands (total).*paired t-test, n=4 independent experiments. Note that anti-DYK antibody detected 2 distinct bands that differ by ˜19 kDa whereas exon 14 encodes 40 amino acids (<5 kDa). The difference is from the heavy glycosylation of exon 14 as deglycosylation of lysates with PNGase rendered the bands indistinguishable in size.

FIGS. 17A-C show comparison of RNA-seq with genomic variant calls and population stratification. FIG. 17A shows allele frequencies of the 641 variants tested for eQTL presence as called by RNA-seq in the NL cohort (x-axis) versus allele frequencies as reported in the Netherlands genome database (GoNL (Genome of the Netherlands Consortium, Francioli L C, Menelaou A, et. al. Nat Genet. 2014; 46:818-825)); Pearson cor=0.93 (p<2.2e-16). Allele frequencies for cohort 1 compared to those reported in 1000 genomes database (Gibbs R A, et al. Nature. 2015; 526:68-74) were also assessed but are not shown: cor=0.87 (p<2.2e-16) for white individuals in cohort 1 RNA-seq calls vs European superpopulation genomes; cor=0.89 (p<2.2e-16) for black/African American individuals in cohort1 RNA-seq calls vs African superpopulation genomes. FIG. 17B shows PCA analysis comparing allele frequencies of the 641 variants as called by RNA-seq in Black/African American (AA) or white individuals in cohort 1, or the NL cohort, with allele frequencies reported in GoNL and 1000 genomes database superpopulations: East Asian (EAS), South Asian (SAS), Ad Mixed American (AMR), European (EUR), or African (AFR). FIG. 17C shows MDS analysis (Purcell S, et al. Am J Hum Genet. 2007; 81:559-75; and Chang C C, et al. Gigascience. 2015; 4:7) of population structure for each individual in cohort 1 and the NL cohort anchored to individuals from 1000 genomes. 1994 variants co-identified in the RNA-seq cohorts and 1000 genomes were used in the analysis. Results indicate that cohort 1 white individuals and the NL cohort RNA-seq individuals cluster almost exclusively with the EUR genome superpopulation, whereas the cohort 1 black/African American and other/unknown RNA-seq individuals cluster with the AFR superpopulation, and suggest admixture. The top three MDS components are plotted. Zoom-boxes are included for clarity where there is a high density of cohort1 white, NL cohort, and EUR individuals.

### SUMMARY

Disclosed herein are methods for making a transcriptome-wide expression profile of a biological sample, said methods comprising: a) measuring the expression levels of one or more genes present in a first biological sample, wherein said measuring comprises RNA sequencing; b) determining the expression levels of the genes from the measured expression levels obtained in step a); c) combining the results of steps a) and b) to produce a transcriptome-wide expression profile; and d) providing the transcriptome-wide expression profile as a data set, wherein the first biological sample consists of isolated platelets.

### DETAILED DESCRIPTION

The present disclosure can be understood more readily by reference to the following detailed description of the invention, the figures and the examples included herein.

Before the present methods and gene expression panels are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In some aspects, a subject is a mammal. In some aspects, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for a disease, such as, for example, prior to the administering step.

In some aspects, the terms “patient,” “subject,” “individual,” and the like are used interchangeably herein, and refer to any animal, or cells thereof whether in vitro or in situ, amenable to the methods described herein. In some aspects, the patient, subject or individual is a human.

As used herein, the term “comprising” can include the aspects “consisting of” and “consisting essentially of.”

As used herein, the term “normal” or “healthy” refers to an individual, a sample or a subject that does not have a disease or disorder or does not have an increased susceptibility of developing a disease or disorder.

As used herein, the term “susceptibility” refers to the likelihood of a subject being clinically diagnosed with a disease. For example, a human subject with an increased susceptibility for a disease can refer to a human subject with an increased likelihood of a subject being clinically diagnosed with a disease.

As used herein, the term “polypeptide” refers to any peptide, oligopeptide, polypeptide, gene product, expression product, or protein. A polypeptide is comprised of consecutive amino acids. The term “polypeptide” encompasses naturally occurring or synthetic molecules. As used herein, the term “amino acid sequence” refers to a list of abbreviations, letters, characters or words representing amino acid residues.

As used herein, the terms “peptide,” “polypeptide,” and “protein” are used interchangeably, and refer to a compound comprising amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the term “gene” refers to a region of DNA encoding a functional RNA or protein. “Functional RNA” refers to an RNA molecule that is not translated into a protein. Generally, the gene symbol is indicated by using italicized styling while the protein symbol is indicated by using non-italicized styling.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

Nucleic acids may also include any polymer or oligomer of pyrimidine and purine bases, preferably cytosine, thymine, and uracil, and adenine and guanine, respectively. Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferably at least 8, 15 or 25 nucleotides in length, but may be up to 50, 100, 1000, or 5000 nucleotides long or a compound that specifically hybridizes to a polynucleotide. Polynucleotides include sequences of deoxyribonucleic acid (DNA) or ribonucleic acid (RNA) or mimetics thereof which may be isolated from natural sources, recombinantly produced or artificially synthesized. A further example of a polynucleotide of the present invention may be a peptide nucleic acid (PNA). (See U.S. Pat. No. 6,156,501 which is hereby incorporated by reference in its entirety.) The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this disclosure. It will be understood that when a nucleotide sequence is represented herein by a DNA sequence (e.g., A, T, G, and C), this also includes the corresponding RNA sequence (e.g., A, U, G, C) in which “U” replaces “T”.

As used herein, “polynucleotide” includes cDNA, RNA, DNA/RNA hybrid, antisense RNA, ribozyme, genomic DNA, synthetic forms, and mixed polymers, both sense and antisense strands, and may be chemically or biochemically modified to contain non-natural or derivatized, synthetic, or semi-synthetic nucleotide bases. Also, contemplated are alterations of a wild type or synthetic gene, including but not limited to deletion, insertion, substitution of one or more nucleotides, or fusion to other polynucleotide sequences.

By “isolated polypeptide” or “purified polypeptide” is meant a polypeptide (or a fragment thereof) that is substantially free from the materials with which the polypeptide is normally associated in nature. The polypeptides of the invention, or fragments thereof, can be obtained, for example, by extraction from a natural source (for example, a mammalian cell), by expression of a recombinant nucleic acid encoding the polypeptide (for example, in a cell or in a cell-free translation system), or by chemically synthesizing the polypeptide. In addition, polypeptide fragments may be obtained by any of these methods, or by cleaving full length polypeptides.

By “isolated nucleic acid” or “purified nucleic acid” is meant DNA that is free of the genes that, in the naturally-occurring genome of the organism from which the DNA of the invention is derived, flank the gene. The term therefore includes, for example, a recombinant DNA which is incorporated into a vector, such as an autonomously replicating plasmid or virus; or incorporated into the genomic DNA of a prokaryote or eukaryote (e.g., a transgene); or which exists as a separate molecule (for example, a cDNA or a genomic or cDNA fragment produced by PCR, restriction endonuclease digestion, or chemical or in vitro synthesis). It also includes a recombinant DNA which is part of a hybrid gene encoding additional polypeptide sequence. The term “isolated nucleic acid” also refers to RNA, e.g., an mRNA molecule that is encoded by an isolated DNA molecule, or that is chemically synthesized, or that is separated or substantially free from at least some cellular components, for example, other types of RNA molecules or polypeptide molecules.

By “specifically binds” is meant that an antibody recognizes and physically interacts with its cognate antigen and does not significantly recognize and interact with other antigens; such an antibody may be a polyclonal antibody or a monoclonal antibody, which are generated by techniques that are well known in the art.

By “probe,” “primer,” or oligonucleotide is meant a single-stranded DNA or RNA molecule of defined sequence that can base-pair to a second DNA or RNA molecule that contains a complementary sequence (the “target”). The stability of the resulting hybrid depends upon the extent of the base-pairing that occurs. The extent of base-pairing is affected by parameters such as the degree of complementarity between the probe and target molecules and the degree of stringency of the hybridization conditions. The degree of hybridization stringency is affected by parameters such as temperature, salt concentration, and the concentration of organic molecules such as formamide, and is determined by methods known to one skilled in the art. Probes or primers specific for a particular nucleic acid (for example, genes and/or mRNAs) can have at least 80%-90% sequence complementarity, preferably at least 91%-95% sequence complementarity, more preferably at least 96%-99% sequence complementarity, and most preferably 100% sequence complementarity to the region of the nucleic acid to which they hybridize. Probes, primers, and oligonucleotides may be detectably-labeled, either radioactively, or non-radioactively, by methods well-known to those skilled in the art. Probes, primers, and oligonucleotides are used for methods involving nucleic acid hybridization, such as: nucleic acid sequencing, reverse transcription and/or nucleic acid amplification by the polymerase chain reaction, single stranded conformational polymorphism (SSCP) analysis, restriction fragment polymorphism (RFLP) analysis, Southern hybridization, Northern hybridization, in situ hybridization, electrophoretic mobility shift assay (EMSA).

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences.

The term “primer” refers to an oligonucleotide capable of acting as a point of initiation of synthesis along a complementary strand when conditions are suitable for synthesis of a primer extension product. The synthesizing conditions include the presence of four different deoxyribonucleotide triphosphates and at least one polymerization-inducing agent such as reverse transcriptase or DNA polymerase. These are present in a suitable buffer, which may include constituents which are co-factors or which affect conditions such as pH and the like at various suitable temperatures. A primer is preferably a single strand sequence, such that amplification efficiency is optimized, but double stranded sequences can be utilized.

By “specifically hybridizes” is meant that a probe, primer, or oligonucleotide recognizes and physically interacts (that is, base-pairs) with a substantially complementary nucleic acid (for example, a nucleic acid of interest) under high stringency conditions, and does not substantially base pair with other nucleic acids.

By “high stringency conditions” is meant conditions that allow hybridization comparable with that resulting from the use of a DNA probe of at least 40 nucleotides in length, in a buffer containing 0.5 M NaHPO₄, pH 7.2, 7% SDS, 1 mM EDTA, and 1% BSA (Fraction V), at a temperature of 65° C., or a buffer containing 48% formamide, 4.8×SSC, 0.2 M Tris-Cl, pH 7.6, 1×Denhardt's solution, 10% dextran sulfate, and 0.1% SDS, at a temperature of 42° C. Other conditions for high stringency hybridization, such as for PCR, Northern, Southern, or in situ hybridization, DNA sequencing, etc., are well-known by those skilled in the art of molecular biology. (See, for example, F. Ausubel et al., Current Protocols in Molecular Biology, John Wiley & Sons, New York, N.Y., 1998).

The term “abnormal” is used to refer to organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “amplification” refers to the operation by which the number of copies of a target nucleotide sequence present in a sample is multiplied.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab and F(ab)2, as well as single chain antibodies (scFv), heavy chain antibodies, such as camelid antibodies, synthetic antibodies, chimeric antibodies, and humanized antibodies (Harlow et al., 1999, Using Antibodies: A Laboratory Manual, Cold Spring Harbor Laboratory Press, N.Y.; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

As used herein, an “immunoassay” refers to any binding assay that uses an antibody capable of binding specifically to a target molecule to detect and quantify the target molecule.

The term “coding sequence,” as used herein, refers to a sequence of a nucleic acid or its complement, or a part thereof that can be transcribed and/or translated to produce the mRNA and/or the polypeptide or a fragment thereof. Coding sequences include exons in a genomic DNA or immature primary RNA transcripts, which are joined together by the cell's biochemical machinery to provide a mature mRNA. The anti-sense strand is the complement of such a nucleic acid, and the coding sequence can be deduced therefrom. In contrast, the term “non-coding sequence,” as used herein, refers to a sequence of a nucleic acid or its complement, or a part thereof that is not translated into amino acid in vivo, or where tRNA does not interact to place or attempt to place an amino acid. Non-coding sequences include both intron sequences in genomic DNA or immature primary RNA transcripts, and gene-associated sequences such as promoters, enhancers, silencers, and the like.

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the term “diagnosis” refers to the determination of the presence of a disease or disorder. In some aspects, methods for making a diagnosis are provided which permit determination of the presence of a particular disease or disorder.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the Tm of the formed hybrid, and the G:C ratio within the nucleic acids. A single molecule that contains pairing of complementary nucleic acids within its structure is said to be “self-hybridized.” A single DNA molecule with internal complementarity could assume a variety of secondary structures including loops, kinks or, for long stretches of base pairs, coils.

“Instructional material,” as that term is used herein, includes a publication, a recording, a diagram, or any other medium of expression which can be used to communicate the usefulness of the nucleic acid, peptide, and/or compound of the invention in the kit for identifying, diagnosing or alleviating or treating the various diseases or disorders recited herein. Optionally, or alternately, the instructional material may describe one or more methods of identifying, diagnosing or alleviating the diseases or disorders in a cell or a tissue of a subject. The instructional material of the kit may, for example, be affixed to a container that contains one or more components of the invention or be shipped together with a container that contains the one or more components of the invention. Alternatively, the instructional material may be shipped separately from the container with the intention that the recipient uses the instructional material and the components cooperatively.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “label” as used herein refers to a detectable compound or composition that is conjugated directly or indirectly to a probe to generate a “labeled” probe. The label may be detectable by itself (e.g., radioisotope labels or fluorescent labels) or, in the case of an enzymatic label, may catalyze chemical alteration of a substrate compound or composition that is detectable (e.g., avidin-biotin). In some instances, primers can be labeled to detect a PCR product.

The terms “microarray” and “array” refers broadly to “DNA microarrays,” “DNA chip(s),” “protein microarrays” and “protein chip(s)” and encompasses all art-recognized solid supports, and all art-recognized methods for affixing nucleic acid, peptide, and polypeptide molecules thereto. Preferred arrays typically comprise a plurality of different nucleic acid or peptide probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 5,800,992, 6,040,193, 5,424,186 and Fodor et al., 1991, Science, 251:767-777, each of which is incorporated by reference in its entirety for all purposes. Arrays may generally be produced using a variety of techniques, such as mechanical synthesis methods or light directed synthesis methods that incorporate a combination of photolithographic methods and solid phase synthesis methods. Techniques for the synthesis of these arrays using mechanical synthesis methods are described in, e.g., U.S. Pat. Nos. 5,384,261, and 6,040,193, which are incorporated herein by reference in their entirety for all purposes. Although a planar array surface is preferred, the array may be fabricated on a surface of virtually any shape or even a multiplicity of surfaces. Arrays may be nucleic acids on beads, gels, polymeric surfaces, fibers such as fiber optics, glass or any other appropriate substrate. (See U.S. Pat. Nos. 5,770,358, 5,789,162, 5,708,153, 6,040,193 and 5,800,992, which are hereby incorporated by reference in their entirety for all purposes.) Arrays may be packaged in such a manner as to allow for diagnostic use or can be an all-inclusive device; e.g., U.S. Pat. Nos. 5,856,174 and 5,922,591 incorporated in their entirety by reference for all purposes. Arrays are commercially available from, for example, Affymetrix (Santa Clara, Calif.) and Applied Biosystems (Foster City, Calif.), and are directed to a variety of purposes, including genotyping, diagnostics, mutation analysis, marker expression, and gene expression monitoring for a variety of eukaryotic and prokaryotic organisms. The number of probes on a solid support may be varied by changing the size of the individual features. In some aspects, the feature size is 20 by 25 microns square, in other aspects, features may be, for example, 8 by 8, 5 by 5 or 3 by 3 microns square, resulting in about 2,600,000, 6,600,000 or 18,000,000 individual probe features.

Assays for amplification of the known sequence are also disclosed. For example primers for PCR may be designed to amplify regions of the sequence. For RNA, a first reverse transcriptase step may be used to generate double stranded DNA from the single stranded RNA. The array may be designed to detect sequences from an entire genome; or one or more regions of a genome, for example, selected regions of a genome such as those coding for a protein or RNA of interest; or a conserved region from multiple genomes; or multiple genomes, arrays and methods of genetic analysis using arrays is described in Cutler, et al., 2001, Genome Res. 11(11): 1913-1925 and Warrington, et al., 2002, Hum Mutat 19:402-409 and in US Patent Pub No 20030124539, each of which is incorporated herein by reference in its entirety.

As used herein, the term “polymerase chain reaction” (“PCR”) refers to the method of K. B. Mullis (U.S. Pat. Nos. 4,683,195 4,683,202, and 4,965,188, hereby incorporated by reference), which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. As used herein, the terms “PCR product,” “PCR fragment,” “amplification product” or “amplicon” refer to the resultant mixture of compounds after two or more cycles of the PCR steps of denaturation, annealing and extension are complete. These terms encompass the case where there has been amplification of one or more segments of one or more target sequences.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “amplification” refers to the operation by which the number of copies of a target nucleotide sequence present in a sample is multiplied.

Platelets are abundant, accessible blood cells that are increasingly used in gene expression studies. Like nucleated cells, platelets possess a diverse portfolio of RNAs, including coding mRNAs, small non-coding RNAs, lncRNAs, and others (Rowley J W, et al. Blood. 2011; 118:e101-e111; Bray P F, et al. BMC Genomics. 2013; 14:1; and Gnatenko D V, et al. Blood. 2003; 101:2285-931-3). Yet their anucleate nature offers advantages over nucleated cells for studying gene expression. For one, ex vivo handling (cell isolation method, processing time, buffers, etc.) of nucleated cells can immediately affect the expression of thousands of transcripts (Beliakova-Bethell N, et al. Cytometry A. 2014; 85:94-104; Bhattacharjee J, et al. F1000Research. 2017; 6:2045; and Baechler E C, et al. Genes Immun. 2004; 5:347-534-6). On the other hand, platelets are transcriptionally unaffected by isolation (Angénieux C, et al. PLoS One. 2016; 11:e0148064; and Best M G, et. al. Cancer Cell. 2015; 28:666-676), allowing capture of the native in vivo gene expression signature. These attractive features render platelets an excellent choice for RNA diagnostics and gene expression studies.

Platelets have been used in GWAS, gene-phenotype (Kondkar A A, et al. J Thromb Haemost. 2010; 8:369-78; and Edelstein L C, et al. Nat Med. 2013; 19:1609-16), diagnostic (Best M G, et al. Cancer Res. 2018; 78:3407-3412), and differential expression studies with an emphasis on RNA abundance to elucidate mediators of platelet reactivity in health and disease (Schubert S, et al. Blood. 2014; 124:493-502). Genetic modifiers of RNA abundance in platelets, called expression quantitative trait loci (eQTL), have also been described (Simon L M, et al. Am J Hum Genet. 2016; 98:883-97; and Kong X, et al. Thromb Haemost. 2017; 117:962-970). eQTLs are DNA sequence variants associated with gene expression that affect nearby (cis-) or remote (trans-) genes in a cell type specific manner. eQTLs are particularly important in genetic studies because they provide an intermediate and mechanistic link between a phenotype and gene association.

Beyond RNA abundance, it is now known that platelets and megakaryocytes harbor alternative structural features of RNA, including alternative start and stop sites, and alternative splicing (Schubert S, et al. Blood. 2014; 124:493-502), that diversify the transcriptome and proteome (Nassa G, et al. Sci Rep. 2018; 8:498; and Schwertz H, et al. J Exp Med. 2006; 203:2433-40) and alter cellular function. In platelets, activation induces RNA splicing, and thereby modulates functional protein expression (Nassa G, et al. Sci Rep. 2018; 8:498; and Denis M M, et al. Cell. 2005; 122:379-91). It is probable that genetic variants called splice QTLs (sQTLs) also influence basal and activation dependent RNA splicing levels in platelets. However, sQTLs for platelets have not yet been described.

Other major knowledge gaps exist regarding RNA abundance and structure in platelets. Most platelet studies have been cross-sectional, examining gene expression at a single time point. Yet, gene expression can vary both between-individuals and within-individuals over time. Hormonal changes, circadian rhythm, inflammation, diet, and aging are examples of environmental cues that might alter gene expression within healthy individuals (Bryois J, et al. Genome Res. 2017; 27:545-552; Waaseth M, et al. BMC Med Genomics. 2011; 4:29; and Arnardottir E S, et al. Sleep. 2014; 37:1589-600). Such normal changes in gene expression can mask the ability to detect signal in differential gene expression, diagnostic, and genetic studies, and confound their analysis. Thus, understanding within individual versus between individual variation in gene expression is important for the design and interpretation of gene expression studies, and can be used to prioritize candidates in genetic studies.

With regard to genetic studies, several reports have suggested using repeatability to identify eQTL genes (Barendse W. BMC Genomics. 2011; 12:232; Carlborg O, et al. Bioinformatics. 2004; 21:2383-93; and Hoffman G E, Schadt E E. BMC Bioinformatics. 2016; 17:48321-23). In vivo repeatability can only be calculated from multiple samplings from the same individual, and refers to the proportion of variation attributed to between-individual versus within-individual variation (Lessells C M, Boag P T. Auk. 1987; 104:116-121). In the straightforward view, repeatability sets an upper-bound to broad sense heritability (Dohm M R. Funct Ecol. 2002; 16:273-280): if between individual differences are not repeatable because of low between, and/or high within-individual variation, there will be insufficient power to detect heritable, genetic signal. For this reason, it has been recommended to measure the repeatability of a trait before performing GWAS21. Carlborg et. al. (Carlborg O, et al. Bioinformatics. 2004; 21:2383-93) found that censoring mouse eQTL data on repeatability was an effective method for prioritizing transcripts with a high a priori likelihood of successful eQTL identification. Hoffman et. al. (Hoffman G E, Schadt E E. BMC Bioinformatics. 2016; 17:483) also demonstrated the potential use of within-individual technical variation to narrow candidates and facilitate eQTL prediction, although this study employed single time point replicates. Together, these studies imply that longitudinal analysis of gene expression may facilitate prospective eQTL (and sQTL) gene discovery. Surprisingly, longitudinal analyses of gene expression are scarce in primary cells from healthy individuals, and absent for platelets. The repeatability of alternative splicing over time has not been established for any primary cell type. Disclosed herein are methods for making a transcriptome-wide expression profile of a biological sample that previously has been absent in the art. For example, disclosed herein are methods for making a transcriptome-wide expression profile of a biological sample, said method comprising: a) measuring the expression levels of one or more genes present in a first biological sample, wherein said measuring comprises RNA sequencing; b) determining the expression levels of the genes from the measured expression levels obtained in step a); c) combining the results of steps a) and b) to produce a transcriptome-wide expression profile; and d) providing the transcriptome-wide expression profile as a data set, wherein the first biological sample consists of isolated platelets.

Disclosed herein is the finding that the platelet transcriptome is generally stable over 4 years in healthy individuals providing a longitudinal reference for disease diagnostics. Platelet gene expression signatures have recently been used to accurately classify and diagnose cancers. Changes in the platelet transcriptome, compared to healthy individuals, have also been associated with numerous other diseases (sepsis, influenza infection, lupus, and acute myocardial infarction), raising the possibility for platelet gene expression signatures to diagnose and classify a variety of diseases.

Excessive noise is a known limitation for the use of gene expression signatures for diagnostics, including for platelet gene expression based diagnostics. Most gene expression based diagnostics compare gene expression within a disease to healthy controls. Another approach to gene expression based diagnostics, which minimizes environmental noise and internally corrects for between individual (i.e., genetic) variation, is to compare the gene signature of an individual with disease to the gene signature from same individual at a time when the individual was healthy, i.e., longitudinal sampling. This approach relies on the assumption that the gene signature is relatively stable over time in healthy individuals, or that changes over time in healthy individuals are known and consistent. However, the extent of variation in gene expression over time is unknown for most cell types including platelets. A reference gene signature for longitudinal studies in platelets is not currently available. To solve this problem, gene expression was assessed in platelets up to 4 years in healthy individuals, identifying a transcriptome-wide expression profile of healthy individuals, which includes the least and most stable genes and splicing events within platelets from healthy individuals over time. The transcriptome-wide expression profile can be used as a reference for platelet differential gene expression analysis and diagnostics by providing the amount of expected variation within healthy individuals over time. Deviations from this reference can indicate disease.

As described herein, a transcriptome-wide expression profile can be used as a reference for platelet differential gene expression analysis and diagnostics by providing the amount of expected variation within a single healthy individual or multiple healthy individuals over time. In some aspects, a transcriptome-wide expression profile as disclosed herein can be used as a reference for platelet differential gene expression analysis and diagnostics by providing the amount of expected variation within a single individual or multiple individuals with diseases over time. In some aspects, a transcriptome-wide expression profile as disclosed herein can be used as a reference for platelet differential gene expression analysis and diagnostics by providing the amount of expected variation within a single individual or multiple individuals wherein the single or multiple individuals are healthy, have disease, or a combination of healthy and disease. Deviations from this reference may useful as a regular screen to differentiate healthy versus disease states. For example, provided herein are within- and between-individual variation, and repeatability measurements for the least and most variable genes expressed in platelets. These can be used as a reference for future disease studies that assess gene expression especially longitudinally, i.e., compare gene expression in samples from a healthy individual versus samples isolated longitudinally from the same individual when they have a suspected disease. Demonstrated herein is the repeatability of gene expression in platelets for predicting eQTL presence. This list can be used to narrow the number of genetic variants that might be useful for inclusion in disease diagnostic signatures.

Advantages of the methods disclosed herein include but are not limited to providing a more precise “healthy” reference for diagnostic testing, and individual patients can be tested over time and serve as their own reference. The methods disclosed herein can also be used as a standard for other diagnostic tests, for example, gene testing. Further, using a longitudinal reference can reduce the cost of developing a disease diagnostic signature that would otherwise require sampling gene expression longitudinally in additional healthy individuals.

Disclosed herein are transcriptome-wide expression profiles and methods of generating the same. Disclosed herein are transcriptome-wide expression profiles, wherein the profile is derived from the platelet sample. Disclosed herein is a transcriptome-wide expression profile for platelets. For example, disclosed herein is a transcriptome-wide expression profile obtained from healthy individuals. This transcriptome-wide expression profile was derived by sampling the same individuals over a period of time (4 years) allowing for the transcriptome-wide expression profile to account for variations within an individual over time. In some aspects, a transcriptome-wide expression profile can be obtained from individuals with a particular illness, condition, disease or injury or a set of particular illnesses, conditions, diseases or injuries. Other reference gene signatures rely on the sampling of many different people at a single time point to come up with an average signature. This longitudinal method allows for examination of natural fluctuation in the expression of individual genes within a single individual allowing for the identification of the most and least stable genes over time.

A transcriptome-wide expression profile can be a gene signature or gene expression signature that is a single or combined group of genes in a cell with a uniquely characteristic pattern of gene expression that occurs as a result of an altered or unaltered biological process or pathogenic medical condition. The clinical applications of transcriptome-wide expression profiles breakdown into prognostic, diagnostic and predictive signatures. The phenotypes that may theoretically be defined by a transcriptome-wide expression profile range from those that predict the survival or prognosis of an individual with a disease, those that are used to differentiate between different subtypes of a disease, to those that predict activation of a particular pathway. In summary, the disclosed methods address the need for transcriptome-wide expression profiles for longitudinal studies of platelet gene signatures. The methods disclosed herein encompass platelet RNA gene signatures or maps for disease diagnosis, and as such, can be used as a highly sensitive analysis method in for example, liquid biopsy, to analyze tumor components in body fluids such as blood.

Disclosed herein are methods of using a transcriptome-wide expression profile (within- and between-individual variation) as a reference for platelet differential gene expression analysis and diagnostics by providing the amount of expected variation with healthy individuals over time.

Disclosed herein are methods of using a transcriptome-wide expression profile (within- and between-individual variation) to compare gene expression in samples from healthy individuals with samples isolated longitudinally from the same individual when they have a suspected disease.

Disclosed herein are methods of using a transcriptome-wide expression profile (within- and between-individual variation) to predict expression of quantitative trait loci (eQTL) and splice quantitative trait loci (sQTL) presence over time.

Disclosed herein are methods of using a transcriptome-wide expression profile (within- and between-individual variation) to determine expression of eQTL genes.

Methods for Making a Transcriptome-Wide Expression Profile

Disclosed herein are methods for making a transcriptome-wide expression profile of a biological sample, said methods comprising: a) measuring the expression levels of one or more genes present in a first biological sample, wherein said measuring comprises RNA sequencing; b) determining the expression levels of the genes from the measured expression levels obtained in step a); c) combining the results of steps a) and b) to produce a transcriptome-wide expression profile; and d) providing the transcriptome-wide expression profile as a data set, wherein the first biological sample consists of isolated platelets. In some aspects, the methods can be repeated at least once. In some aspects, the methods can be repeated on a second biological sample, wherein the second biological sample consists of isolated platelets. In some aspects, the first and second biological samples can be obtained from the same subject or from different subjects. In some aspects, steps a)-d) can be repeated on each biological sample. In some aspects, the transcriptome-wide expression profile of the subjects can be compared. In some aspects, the first and second biological samples can be obtained from the same subject at a first time point and a second time point. In some aspects, the first and second biological samples can be obtained from different subjects, further comprising obtaining additional biological samples from the subjects, wherein the additional biological samples are obtained at different time points. In some aspects, the first and second time points can be different time points. In some aspects, the transcriptome-wide expression profile from the first time point can be compared to the transcriptome-wide expression profile from the second time point. In some aspects, said methods further comprise repeating the steps thereof until a validated transcriptome-wide expression profile can be identified. In some aspects, the expression levels can be measured on a device selected from the group consisting of a microarray, a bead array, a liquid array, and a nucleic-acid sequence.

Disclosed herein are methods for making a transcriptome-wide expression profile of a biological sample. In some aspects, the methods can comprise measuring the expression levels of one or more genes present in a first biological sample. In some aspects, said measuring comprises RNA sequencing. In some aspects, the methods can comprise determining the expression levels of the genes from the measured expression levels obtained in the first biological sample, wherein said measuring comprises RNA sequencing. In some aspects, the methods can comprise combining the results of measuring the expression levels of the genes present in the first biological sample and determining the expression levels of the genes from the measures expression levels to produce a transcriptome-wide expression profile. In some aspects, the method can further provide the transcriptome-wide expression profile as a data set. In some aspects, the first biological sample can consist of isolated platelets. In some aspects, the methods can be repeated at least once. In some aspects, the methods can be repeated on a second biological sample, wherein the second biological sample consists of isolated platelets. In some aspects, the first and second biological samples can be obtained from the same subject or from different subjects. In some aspects, steps a)-d) can be repeated on each biological sample. In some aspects, the transcriptome-wide expression profile of the subjects can be compared. In some aspects, the first and second biological samples can be obtained from the same subject at a first time point and a second time point. In some aspects, the first and second biological samples can be obtained from different subjects, further comprising obtaining additional biological samples from the subjects, wherein the additional biological samples are obtained at different time points. In some aspects, the first and second time points can be different time points. In some aspects, the transcriptome-wide expression profile from the first time point can be compared to the transcriptome-wide expression profile from the second time point. In some aspects, said methods further comprise repeating the steps thereof until a validated transcriptome-wide expression profile can be identified. In some aspects, the expression levels can be measured on a device selected from the group consisting of a microarray, a bead array, a liquid array, and a nucleic-acid sequence.

Disclosed herein are methods for making a transcriptome-wide expression profile of a biological sample. Disclosed herein are methods for making a transcriptome-wide expression profile of a biological sample, said method comprising: a) measuring the expression levels of one or more genes present in a first biological sample, wherein said measuring comprises RNA sequencing; b) determining the expression levels of the genes from the measured expression levels obtained in step a); c) combining the results of steps a) and b) to produce a transcriptome-wide expression profile; and d) providing the transcriptome-wide expression profile as a data set, wherein the first biological sample consists of isolated platelets. In some aspects, the steps a)-d) can be repeated on each biological sample. In some aspects, the transcriptome-wide expression profile of the subjects can be compared. In some aspects, the first and second biological samples can be obtained from the same subject at a first time point and a second time point. In some aspects, the first and second biological samples can be obtained from different subjects, further comprising obtaining additional biological samples from the subjects, wherein the additional biological samples are obtained at different time points. In some aspects, the first and second time points can be different time points. In some aspects, the transcriptome-wide expression profile from the first time point can be compared to the transcriptome-wide expression profile from the second time point. In some aspects, said methods further comprise repeating the steps thereof until a validated transcriptome-wide expression profile can be identified. In some aspects, the expression levels can be measured on a device selected from the group consisting of a microarray, a bead array, a liquid array, and a nucleic-acid sequence.

Also disclosed herein are methods of identifying gene expression differences between two transcriptome-wide expression profiles, the methods comprising: determining one or more variations in subject's platelet transcriptome using the method for making a transcriptome-wide expression profile of a biological sample, said method comprising: a) measuring the expression levels of one or more genes present in a first biological sample, wherein said measuring comprises RNA sequencing; b) determining the expression levels of the genes from the measured expression levels obtained in step a); c) combining the results of steps a) and b) to produce a transcriptome-wide expression profile; and d) providing the transcriptome-wide expression profile as a data set, wherein the first biological sample consists of isolated platelets, wherein the method is performed at different time points, thereby identifying a gene expression difference; and comparing said time-dependent changes from the subject to a reference, wherein the method is performed at different time points, thereby identifying a gene expression difference. In some aspects, the methods can be repeated at least once. In some aspects, the methods can be repeated on a second biological sample, wherein the second biological sample consists of isolated platelets. In some aspects, the first and second biological samples can be obtained from the same subject or from different subjects. In some aspects, steps a)-d) can be repeated on each biological sample. In some aspects, the transcriptome-wide expression profile of the subjects can be compared. In some aspects, the first and second biological samples can be obtained from the same subject at a first time point and a second time point. In some aspects, the first and second biological samples can be obtained from different subjects, further comprising obtaining additional biological samples from the subjects, wherein the additional biological samples are obtained at different time points. In some aspects, the first and second time points can be different time points. In some aspects, the transcriptome-wide expression profile from the first time point can be compared to the transcriptome-wide expression profile from the second time point. In some aspects, said methods further comprise repeating the steps thereof until a validated transcriptome-wide expression profile can be identified. In some aspects, the expression levels can be measured on a device selected from the group consisting of a microarray, a bead array, a liquid array, and a nucleic-acid sequence.

Further disclosed herein are methods of measuring time dependent gene expression differences in platelets from a subject, the methods comprising determining one or more variations in subject's platelet transcriptome using the method for making a transcriptome-wide expression profile of a biological sample, said method comprising: a) measuring the expression levels of one or more genes present in a first biological sample, wherein said measuring comprises RNA sequencing; b) determining the expression levels of the genes from the measured expression levels obtained in step a); c) combining the results of steps a) and b) to produce a transcriptome-wide expression profile; and d) providing the transcriptome-wide expression profile as a data set, wherein the first biological sample consists of isolated platelets, wherein the method is performed at different time points, thereby identifying a gene expression difference; and comparing said time-dependent changes from the subject to a reference. In some aspects, the methods can be repeated at least once. In some aspects, the methods can be repeated on a second biological sample, wherein the second biological sample consists of isolated platelets. In some aspects, the first and second biological samples can be obtained from the same subject or from different subjects. In some aspects, steps a)-d) can be repeated on each biological sample. In some aspects, the transcriptome-wide expression profile of the subjects can be compared. In some aspects, the first and second biological samples can be obtained from the same subject at a first time point and a second time point. In some aspects, the first and second biological samples can be obtained from different subjects, further comprising obtaining additional biological samples from the subjects, wherein the additional biological samples are obtained at different time points. In some aspects, the first and second time points can be different time points. In some aspects, the transcriptome-wide expression profile from the first time point can be compared to the transcriptome-wide expression profile from the second time point. In some aspects, said methods further comprise repeating the steps thereof until a validated transcriptome-wide expression profile can be identified. In some aspects, the expression levels can be measured on a device selected from the group consisting of a microarray, a bead array, a liquid array, and a nucleic-acid sequence.

Biological samples can be of any biological tissue or fluid. Frequently the sample will be a “clinical sample: which is a sample derived from a patient. The biological sample can contain any biological material suitable for detecting the desired biomarkers, and may comprise cellular and/or non-cellular material obtained from the individual. A biological sample can be obtained by appropriate methods, such as, by way of examples, blood draw, fluid draw, or biopsy. Examples of such samples include but are not limited to blood, lymph, urine, gynecological fluids, biopsies, amniotic fluid and smears. Samples that are liquid in nature are referred to herein as “bodily fluids.” Body samples can be obtained from a patient by a variety of techniques including, for example, by scraping or swabbing an area or by using a needle to aspirate bodily fluids. Methods for collecting various body samples are well known in the art. Frequently, a sample will be a “clinical sample,” i.e., a sample derived from a patient. Such samples include, but are not limited to, bodily fluids which may or may not contain cells, e.g., blood (e.g., whole blood, serum or plasma), urine, saliva, tissue or fine needle biopsy samples, and archival samples with known diagnosis, treatment and/or outcome history. In some aspects, the biological sample can comprises blood cell. In some aspects, the sample (or biological sample) can be tissue, blood, serum or plasma. In some aspects, the biological sample can comprise platelets. In some aspects, the sample can be isolated platelets. In some aspects, the biological sample can be from a healthy subject. In some aspects, the biological sample can be from a subject with one or more illnesses, conditions, diseases or injuries.

In some aspects, the methods can further comprise extracting RNA from the isolated platelets.

In some aspects, the transcriptome-wide expression profile from the first time point can be compared to the transcriptome-wide expression profile from the second time point.

In some aspects, the method can further comprise repeating the steps thereof until a validated transcriptome-wide expression profile is identified.

In some aspects, the expression levels can be measured on a device selected from the group consisting of a microarray, a bead array, a liquid array, and a nucleic-acid sequence.

Identifying a Marker or Biomarker

Disclosed herein are methods of identifying a biomarker associated with a disease. In some aspects, the methods can comprise: a) obtaining or having obtained a sample from a subject with the disease, wherein the sample comprises isolated platelets, b) sequencing the isolated platelets in the sample, c) determining gene expression of the sequences in step b), d) repeating steps a), b), c) at different time points, and e) comparing the gene expression at the different points, thereby identifying a biomarker associated with a gene in the subject when a change in the gene expression is at least two standard deviations. In some aspects, a biomarker associated with a gene in the subject can be identified when a change in the gene expression is at least three standard deviations. In some aspects, the methods of identifying a biomarker associated with a disease can be used for diagnosing a disease, assessing the severity of the disease, and assessing the recovery from the disease by detecting differentially expressed biomarkers in a biological sample obtained from a subject as compared to a control or reference sample.

Disclosed herein are methods for making a transcriptome-wide expression profile of a biological sample, said methods comprising: a) measuring the expression levels of one or more genes present in a first biological sample, wherein said measuring comprises RNA sequencing; b) determining the expression levels of the genes from the measured expression levels obtained in step a); c) combining the results of steps a) and b) to produce a transcriptome-wide expression profile; and d) providing the transcriptome-wide expression profile as a data set, wherein the first biological sample consists of isolated platelets, wherein said method can be used to diagnose a disease, assess the severity of the disease, and assess the recovery from the disease by detecting differentially expressed biomarkers in a biological sample obtained from a subject as compared to a control or reference sample.

Disclosed herein are transcriptome-wide expression profiles that can be used for diagnosing a disease, assessing the severity of the disease, and assessing the recovery from the disease by detecting differentially expressed biomarkers in a biological sample obtained from a subject as compared to a control or reference sample.

Disclosed herein are transcriptome-wide expression profiles that are generated by methods disclosed herein that can be used to diagnose a disease, assesse the severity of the disease, and assess the recovery from the disease by detecting differentially expressed biomarkers in a biological sample obtained from a subject as compared to a control or reference sample.

Disclosed herein are methods that can be used to identify differences between transcriptome-wide expression profiles. In some aspects, the methods can include identifying differences between a healthy transcriptome-wide expression profile and a non-healthy (e.g., disease) transcriptome-wide expression profile. In some aspects, the methods of identifying gene expression differences between two transcriptome-wide expression profiles can comprise determining one or more variations in subject's platelet transcriptome using the methods for making a transcriptome-wide expression profile of a biological sample, said methods comprising: a) measuring the expression levels of one or more genes present in a first biological sample, wherein said measuring comprises RNA sequencing; b) determining the expression levels of the genes from the measured expression levels obtained in step a); c) combining the results of steps a) and b) to produce a transcriptome-wide expression profile; and d) providing the transcriptome-wide expression profile as a data set, wherein the first biological sample consists of isolated platelets, wherein the method is performed at different time points, thereby identifying a gene expression difference; and comparing said time-dependent changes from the subject to a reference, wherein the method is performed at different time points, thereby identifying a gene expression difference. In some aspects, the methods can be repeated at least once. In some aspects, the methods can be repeated on a second biological sample, wherein the second biological sample consists of isolated platelets. In some aspects, the first and second biological samples can be obtained from the same subject or from different subjects. In some aspects, steps a)-d) can be repeated on each biological sample. In some aspects, the transcriptome-wide expression profile of the subjects can be compared. In some aspects, the first and second biological samples can be obtained from the same subject at a first time point and a second time point. In some aspects, the first and second biological samples can be obtained from different subjects, further comprising obtaining additional biological samples from the subjects, wherein the additional biological samples are obtained at different time points. In some aspects, the first and second time points can be different time points. In some aspects, the transcriptome-wide expression profile from the first time point can be compared to the transcriptome-wide expression profile from the second time point. In some aspects, said methods further comprise repeating the steps thereof until a validated transcriptome-wide expression profile can be identified. In some aspects, the expression levels can be measured on a device selected from the group consisting of a microarray, a bead array, a liquid array, and a nucleic-acid sequence.

Disclosed herein are methods of detecting differentially expressed markers by nucleic acid microarray. In some aspects, the methods can be used are known to those skilled in the art to detect and to measure the level of differentially expressed marker expression products, such as RNA and protein, to measure the level of one or more differentially expressed marker expression products.

Disclosed herein are methods of detecting or measuring gene expression using methods that focus on cellular components (cellular examination), or methods that focus on examining extracellular components (fluid examination). Because gene expression involves the ordered production of a number of different molecules, a cellular or fluid examination can be used to detect or measure a variety of molecules including RNA, protein, and a number of molecules that can be modified as a result of the protein's function. Typical diagnostic methods focusing on nucleic acids include amplification techniques such as PCR and RT-PCR (including quantitative variants), and hybridization techniques such as in situ hybridization, microarrays, blots, and others. Typical diagnostic methods focusing on proteins include binding techniques such as ELISA, immunohistochemistry, microarray and functional techniques such as enzymatic assays.

Disclosed herein are methods of identifying a variation in a transcriptome in a subject. In some aspects, the methods can comprise: a) obtaining or having obtained a sample from the subject, wherein the sample comprises isolated platelets, b) sequencing the transcriptome isolated platelets in the sample, c) determining gene expression of the transcriptome in step b), d) repeating steps a), b), c) at least once at a different time point, and e) comparing the gene expression determined in step c) and d), thereby identifying a variation in the transcriptome in the subject when a change in the gene expression is at least two standard deviations. In some aspects, a variation in the transcriptome in the subject can be identified when a change in the gene expression is at least three standard deviations. In some aspects, the variation can be a biomarker. In some aspects, the biomarkers can be used for diagnosing a disease, assessing the severity of the disease, and assessing the recovery from the disease by detecting differentially expressed biomarkers in a biological sample obtained from a subject as compared to a control or reference sample. In some aspects, a presence of sequence variation can be detected in a platelet quantitative trait loci (eQTL) gene. In some aspects, a presence of sequence variation can be in a platelet splice quantitative trait loci (sQTL) gene.

Disclosed herein are methods of preparing a data set. In some aspects, the methods can comprise: a) obtaining or having obtained a sample from two or more subjects, wherein the sample comprises isolated platelets; b) sequencing transcriptomes in the sample in a); c) determining gene expression of the transcriptomes from the sequences in b); and d) identifying a variable gene wherein the change in gene expression of the variable gene is at least two standard deviations or identifying a repeatable gene wherein a change in the gene expression of the repeatable gene is less than two standard deviations. In some aspects, a variable gene or a repeatable gene can be identified when a change in the gene expression is at least three standard deviations. In some aspects, the variable gene or the repeatable gene can be a biomarker. In some aspects, the biomarkers can be used for diagnosing a disease, assessing the severity of the disease, and assessing the recovery from the disease by detecting differentially expressed biomarkers in a biological sample obtained from a subject as compared to a control or reference sample.

In some aspects, the data set can be the output generated by the analysis of RNA-Seq reads. The data set can include all or substantially all known features (e.g., exons, introns, etc.) of a reference. The data set can be used to identify biomarkers.

In some aspects, the methods disclosed herein can include a step of aligning sequence reads that are substantially comprehensively represented in an annotated reference. Using alignment algorithms, reads can be rapidly mapped despite the large numbers associated with RNA-Seq results and substantially comprehensive references.

In some aspects, the methods can comprise analyzing a transcriptome by obtaining a plurality of sequence reads from a transcriptome, finding alignments—each with an alignment score that meets a predetermined criteria, and identifying transcripts in the transcriptome. The method is suited for analysis of reads obtained by RNA-Seq. The predetermined criteria may be, for example, the highest-scoring alignment.

In some aspects, the methods and systems disclosed herein can include transcriptome analysis in which a data set can be used as a reference.

In some aspects, the data set can represent substantially all known exons for at least one chromosome. In some aspects, the alignments can be found by comparing each sequence read to at least a majority of the possible paths through the data set. The method may include assembling the plurality of sequence reads into a contig based on the found alignments.

In some aspects, identifying transcripts include identifying known biomarkers and novel biomarkers.

The methods disclosed herein can include identifying expression levels of the transcripts.

In some aspects, the methods disclosed herein can further comprise monitoring disease progression, monitoring residual disease, monitoring therapy, diagnosing a condition, prognosing a condition, or selecting a therapy based on discovered variants or biomarkers.

In some aspects, the methods disclosed herein can further comprise identification of a variant or biomarker that can be followed up through an imaging test (e.g., CT, PET-CT, MRI, X-ray, ultrasound) for localization of the tissue abnormality suspected of causing the identified variant or biomarker.

In some aspects, the methods can be used to identify any gene. In some aspects, the gene can be a heritable gene. In some aspects, any of the methods disclosed here can be repeated a plurality of times. In some aspects, any of the methods disclosed here can be repeated 1, 2, 3, 4, 5, 6, 7, 8, 9, 10 or more times.

The genes identified as being differentially expressed can be assessed in a variety of nucleic acid detection assays to detect or quantify the expression level of a gene or multiple genes in a given sample. For example, traditional Northern blotting, nuclease protection, RT-PCR, microarray, and differential display methods can be used for detecting gene expression levels. Methods for assaying for mRNA include Northern blots, slot blots, dot blots, and hybridization to an ordered array of oligonucleotides. Any method for specifically and quantitatively measuring a specific protein or mRNA or DNA product can be used. However, methods and assays are most efficiently designed with array or chip hybridization-based methods for detecting the expression of a large number of genes. Any hybridization assay format can be used, including solution-based and solid support-based assay formats.

The protein products of the genes identified herein can also be assayed to determine the amount of expression. Methods for assaying for a protein include Western blot, immunoprecipitation, and radioimmunoassay. The proteins analyzed can be localized intracellularly (most commonly an application of immunohistochemistry) or extracellularly (most commonly an application of immunoassays such as ELISA).

In some aspects, the biological sample can obtained from the subject before the onset of a sign or symptom of a disease or injury. For example, in some aspects, the biological sample can obtained about 1 minute, 5 minutes, 10 minutes 30 minutes, 1 hour, 2 hours, 4 hours, 6 hours, 12 hours, 18 hours, 24 hours, 2 days, 3 days, 4 days, 5 days, 6 days, 7 days, 10 days, 2 weeks, 3 weeks, 4 weeks, 1 month, 2 months, 3 months, 4 months, 5 months, 6 months, 9 months, 1 year, 2 years before the onset of a sign or symptom of a disease or injury. In some aspects, the sample can be obtained more than 2 years, 3 years, 4 years, 5 years, 6 years, 7 years, 8 years, 9 years, or 10 years before the onset of a sign or symptom of a disease or injury. In some aspects, the sample can obtained less than 1 minute before the onset of a sign or symptom of a disease or injury. In some aspects, a plurality of biological samples can be obtained at one or more different time points.

In some aspects, the biological sample can obtained from the subject following the onset of a sign or symptom of a disease or injury. For example, in some aspects, the biological sample can obtained about 1 minute, 5 minutes, 10 minutes 30 minutes, 1 hour, 2 hours, 4 hours, 6 hours, 12 hours, 18 hours, 24 hours, 2 days, 3 days, 4 days, 5 days, 6 days, 7 days, 10 days, 2 weeks, 3 weeks, 4 weeks, 1 month, 2 months, 3 months, 4 months, 5 months, 6 months, 9 months, 1 year, 2 years, 3 years, 4 years, 5 years, 6 years, 7 years, 8 years, 9 years, or 10 years following the onset of a sign or symptom of a disease or injury. In certain embodiments, the sample is obtained more than 2 years, 3 years, 4 years, 5 years, 6 years, 7 years, 8 years, 9 years, or 10 years following the onset of a sign or symptom of a disease or injury. In some aspects, the sample can obtained less than 1 minute following the onset of a sign or symptom of a disease or injury. In certain embodiments, a plurality of biological samples can be obtained at one or more different time points.

In some aspects, the methods can further comprise obtaining a second sample from the subject. In some aspects, the subject can be the same subject. In some aspects, the second sample from the same subject can be obtained between 4 months and 4 years. In some aspects, the subject has one or more signs or symptoms of a disease.

Control group samples can either be from a normal or healthy subject or samples from subjects with a known disease or injury. In some aspects, the control sample can be either from subjects who have recovered or have not recovered from a known disease or injury. As described herein, comparison of the expression patterns of the sample to be tested with those of the controls can be used to diagnose a disease or injury, assess the severity of the disease or injury, or assess the recovery from the disease or injury. In some aspects, the control groups are for the purposes of establishing initial cutoffs or thresholds for the assays described herein. Therefore, in some aspects, the systems and methods disclosed herein can be used to diagnose a disease or injury, assess the severity of a disease or injury, or assess the recovery from the disease or injury without the need to compare with a control group.

Methods of Diagnosis

Disclosed herein are methods for diagnosing a disease or injury, assessing disease or injury severity, and assessing recovery from the disease or injury in a subject who has or has not experienced a sign or symptom of a disease or injury by generating a transcriptome-wide expression profile.

In some aspects, the methods for making a transcriptome-wide expression profile of a biological sample can be used for diagnosing a disease or injury, assessing disease or injury severity, and assessing recovery from the disease or injury in a subject who has or has not experienced a sign or symptom of a disease or injury by generating a transcriptome-wide expression profile. In some aspects, the methods for making a transcriptome-wide expression profile of a biological sample can comprise: a) measuring the expression levels of one or more genes present in a first biological sample, wherein said measuring comprises RNA sequencing; b) determining the expression levels of the genes from the measured expression levels obtained in step a); c) combining the results of steps a) and b) to produce a transcriptome-wide expression profile; and d) providing the transcriptome-wide expression profile as a data set, wherein the first biological sample consists of isolated platelets. In some aspects, the first and second biological samples can be obtained from the same subject or from different subjects. In some aspects, steps a)-d) can be repeated on each biological sample. In some aspects, the transcriptome-wide expression profile of the subjects can be compared. In some aspects, the first and second biological samples can be obtained from the same subject at a first time point and a second time point. In some aspects, the first and second biological samples can be obtained from different subjects, further comprising obtaining additional biological samples from the subjects, wherein the additional biological samples are obtained at different time points. In some aspects, the first and second time points can be different time points. In some aspects, the transcriptome-wide expression profile from the first time point can be compared to the transcriptome-wide expression profile from the second time point. In some aspects, said methods further comprise repeating the steps thereof until a validated transcriptome-wide expression profile can be identified. In some aspects, the expression levels can be measured on a device selected from the group consisting of a microarray, a bead array, a liquid array, and a nucleic-acid sequence.

Disclosed herein are methods for making a transcriptome-wide expression profile of a biological sample, said methods comprising: a) measuring the expression levels of one or more genes present in a first biological sample, wherein said measuring comprises RNA sequencing; b) determining the expression levels of the genes from the measured expression levels obtained in step a); c) combining the results of steps a) and b) to produce a transcriptome-wide expression profile; and d) providing the transcriptome-wide expression profile as a data set, wherein the first biological sample consists of isolated platelets, wherein said method can be used to diagnose a disease or injury, assess disease or injury severity, and assessing recovery from the disease or injury in a subject who has or has not experienced a sign or symptom of a disease or injury by generating a transcriptome-wide expression profile.

Disclosed herein are transcriptome-wide expression profiles that can be used for diagnosing a disease, assessing the severity of the disease, and assessing the recovery from the disease by detecting differentially expressed biomarkers in a biological sample obtained from a subject as compared to a control or reference sample.

Disclosed herein are transcriptome-wide expression profiles that are generated by methods disclosed herein that can be used to diagnose a disease, assesse the severity of the disease, and assess the recovery from the disease by detecting differentially expressed biomarkers in a biological sample obtained from a subject as compared to a control or reference sample.

Disclosed herein are methods that can be used to identify differences between transcriptome-wide expression profiles. In some aspects, the methods can include identifying differences between a healthy transcriptome-wide expression profile and a non-healthy (e.g., disease) transcriptome-wide expression profile. In some aspects, the methods of identifying gene expression differences between two transcriptome-wide expression profiles can comprise determining one or more variations in subject's platelet transcriptome using the methods for making a transcriptome-wide expression profile of a biological sample, said methods comprising: a) measuring the expression levels of one or more genes present in a first biological sample, wherein said measuring comprises RNA sequencing; b) determining the expression levels of the genes from the measured expression levels obtained in step a); c) combining the results of steps a) and b) to produce a transcriptome-wide expression profile; and d) providing the transcriptome-wide expression profile as a data set, wherein the first biological sample consists of isolated platelets, wherein the method is performed at different time points, thereby identifying a gene expression difference; and comparing said time-dependent changes from the subject to a reference, wherein the method is performed at different time points, thereby identifying a gene expression difference. In some aspects, the methods can be repeated at least once. In some aspects, the methods can be repeated on a second biological sample, wherein the second biological sample consists of isolated platelets. In some aspects, the first and second biological samples can be obtained from the same subject or from different subjects. In some aspects, steps a)-d) can be repeated on each biological sample. In some aspects, the transcriptome-wide expression profile of the subjects can be compared. In some aspects, the first and second biological samples can be obtained from the same subject at a first time point and a second time point. In some aspects, the first and second biological samples can be obtained from different subjects, further comprising obtaining additional biological samples from the subjects, wherein the additional biological samples are obtained at different time points. In some aspects, the first and second time points can be different time points. In some aspects, the transcriptome-wide expression profile from the first time point can be compared to the transcriptome-wide expression profile from the second time point. In some aspects, said methods further comprise repeating the steps thereof until a validated transcriptome-wide expression profile can be identified. In some aspects, the expression levels can be measured on a device selected from the group consisting of a microarray, a bead array, a liquid array, and a nucleic-acid sequence.

In some aspects, the methods for identifying subjects who have a disease or injury, including those subjects who are asymptomatic or exhibit non-specific indicators of disease or injury by generating transcriptome-wide expression profiles and detecting one or more biomarkers. These biomarkers are also useful for monitoring subjects undergoing treatments and therapies for the disease or injury and/or the disease- or injury-related conditions, and for selecting or modifying therapies and treatments that would be efficacious in subjects having a disease or injury, wherein selection and use of such treatments and therapies. Such treatments can treat the disease or injury by slowing or preventing disease- or injury-associated symptoms.

Disclosed herein are improved methods for the diagnosis and prognosis of disease or injury. The diagnosis or prognosis of a disease or injury can be assessed by measuring one or more of the biomarkers described herein, and comparing the measured values to comparator values, reference values, or index values. Such a comparison can be undertaken with mathematical algorithms or formula in order to combine information from results of multiple individual biomarkers (e.g., a signature, a variant gene) and other parameters into a single measurement or index. Subjects identified as having a disease or injury can optionally be selected to receive treatment regimens, such as administration of therapeutic compounds to prevent, treat or delay the disease- or injury-related symptoms.

Identifying a subject as having a disease or injury within a few hours before or after the onset of a sign or symptom of the disease or injury can allow for the selection and initiation of various therapeutic interventions or treatment regimens in order to delay, reduce or prevent disease- or injury-related symptoms as well as improve recovery. Monitoring the levels of at least one biomarker also allows for the course of treatment to be monitored. For example, a sample can be provided from a subject undergoing treatment regimens or therapeutic interventions. Such treatment regimens or therapeutic interventions can include but is not limited to administration of pharmaceuticals, and treatment with therapeutics or prophylactics used in subjects diagnosed or identified with a disease or injury. Samples can be obtained from the subject at various time points before, during, or after treatment.

The biomarkers (or signatures) identified by the methods disclosed herein can thus be used to generate a biomarker profile or signature of the subject(s): (i) who do not have a disease or injury, (ii) who have a disease or injury or a symptom(s) or a sign(s) of the disease or injury, and/or (iii) who are recovering or have recovered from the disease or injury. The biomarker profile of a subject can be compared to a predetermined or comparator biomarker profile or reference biomarker profile to diagnose the disease or injury, to monitor the progression or rate of progression of disease- or injury-related symptoms or pathology, and to monitor the effectiveness of disease or injury treatments. Data concerning the biomarkers identified by the methods disclosed herein can also be combined or correlated with other data or test results, such as, without limitation, measurements of clinical parameters or other algorithms for disease or injury. Other data includes age, gender, ethnicity, body mass index (BMI), neurological testing, EEG recording data, EKG recording data, imaging results (e.g., CT scan, MRI, angiography), and the like. The data may also comprise subject information such as medical history and any relevant family history.

Disclosed herein are methods that can be used for identifying agents for treating a disease or injury that are appropriate or otherwise customized for a specific subject. In this regard, a test sample from a subject, exposed to a therapeutic agent or a drug, can be taken and the level of one or more biomarkers can be determined. The level of one or more biomarkers can be compared to a sample derived from the subject before and after treatment, or can be compared to samples derived from one or more subjects who have shown improvements in risk factors as a result of such treatment or exposure.

In some aspects, the methods described herein can utilize a biological sample (e.g., platelets), for the detection of one or more biomarkers in the sample. In some aspects, the method comprises detection of one or more biomarkers in a platelet of the subject.

In some aspect, the transcriptome-wide expression profiles generated as disclosed herein can be used for the diagnosis of an illness, condition, disease or injury in a subject. In some aspects, the disease or injury includes but is not limited to infectious diseases, which include but are not limited to bacterial, fungal, viral, and parasitic infectious diseases, and diseases arising from the infection including but not limited to sepsis, severe sepsis, and septic shock, COVID-19, multisystem inflammatory syndrome in children (MIS-C), and systemic inflammation. In some aspects, the disease or injury includes but is not limited to cancer, autoimmune disease, skin diseases, eye disease, endocrine diseases, neurological disorders, and cardiovascular diseases.

In some aspects, the cancer can be a solid tumor cancer, including but not limited to colon, pancreas, brain, bladder, breast, prostate, lung, breast, ovary, uterus, liver, kidney, spleen, thymus, thyroid, nerve tissue, epithelial tissue, lymph node, bone, muscle and skin.

In some aspects, the auto-immune disease can include but is not limited to Achlorhydra Autoimmune Active Chronic Hepatitis; Acute Disseminated Encephalomyelitis; Acute hemorrhagic leukoencephalitis; Addison's Disease; Agammaglobulinemia; Alopecia areata; Amyotrophic Lateral Sclerosis; Ankylosing Spondylitis; Anti-GBM/TBM Nephritis; Antiphospholipid syndrome; Antisynthetase syndrome; polyarticular Arthritis; Atopic allergy; Atopic Dermatitis; Autoimmune Aplastic Anemia; Autoimmune cardiomyopathy; Autoimmune enteropathy; Autoimmune hemolytic anemia; Autoimmune hepatitis; Autoimmune inner ear disease; Autoimmune lymphoproliferative syndrome; Autoimmune peripheral neuropathy; Autoimmune pancreatitis; Autoimmune polyendocrine syndrome; Autoimmune progesterone dermatitis; Autoimmune thrombocytopenic purpura; Autoimmune uveitis; Balo disease/Balo concentric sclerosis; Bechets Syndrome; Berger's disease; Bickerstaffs encephalitis; Blau syndrome; Bullous Pemphigoid; Castleman's disease; Celiac disease; Chagas disease; Chronic Fatigue Immune Dysfunction Syndrome; Chronic inflammatory demyelinating polyneuropathy; Chronic recurrent multifocal osteomyelitis; Chronic lyme disease; Chronic obstructive pulmonary disease; Churg-Strauss syndrome; Cicatricial Pemphigoid; Coeliac Disease; Cogan syndrome; Cold agglutinin disease; Complement component 2 deficiency; Cranial arteritis; CREST syndrome; Crohns Disease; Cushing's Syndrome; Cutaneous leukocytoclastic angiitis; Dego's disease; Dercum's disease; Dermatitis herpetiformis; Dermatomyositis; Diabetes mellitus type 1; Diffuse cutaneous systemic sclerosis; Dressler's syndrome; Discoid lupus erythematosus; Eczema; Endometriosis; Enthesitis-related arthritis; Eosinophilic fasciitis; Eosinophilic gastroenteritis; Epidermolysis bullosa acquisita; Erythema nodosum; Essential mixed cryoglobulinemia; Evan's syndrome; Fibrodysplasia ossificans progressiva; Fibromyalgia/Fibromyositis; Fibrosing aveolitis; Gastritis; Gastrointestinal pemphigoid; Giant cell arteritis; Glomerulonephritis; Goodpasture's syndrome; Graves' disease; Guillain-Barre syndrome; Hashimoto's encephalitis; Hashimoto's thyroiditis; Haemolytic anaemia; Henoch-Schonlein purpura; Herpes gestationis; Hidradenitis suppurativa; Hughes syndrome; Hypogammaglobulinemia; Idiopathic Inflammatory Demyelinating Diseases; Idiopathic pulmonary fibrosis; Idiopathic thrombocytopenic purpura; IgA nephropathy; Inclusion body myositis; Inflammatory demyelinating polyneuopathy; Interstitial cystitis; Irritable Bowel Syndrome (IBS); Juvenile idiopathic arthritis; Juvenile rheumatoid arthritis; Kawasaki's Disease; Lambert-Eaton myasthenic syndrome; Leukocytoclastic vasculitis; Lichen planus; Lichen sclerosus; Linear IgA disease; Lou Gehrig's Disease; Lupoid hepatitis; Lupus erythematosus; Majeed syndrome; Meniere's disease; Microscopic polyangiitis; Miller-Fisher syndrome; Mixed Connective Tissue Disease; Morphea; Mucha-Habermann disease; Muckle-Wells syndrome; Multiple Myeloma; Multiple Sclerosis; Myasthenia gravis; Myositis; Narcolepsy; Neuromyelitis optica; Neuromyotonia; Occular cicatricial pemphigoid; Opsoclonus myoclonus syndrome; Ord thyroiditis; Palindromic rheumatism; PANDAS; Paraneoplastic cerebellar degeneration; Paroxysmal nocturnal hemoglobinuria; Parry Romberg syndrome; Parsonnage-Turner syndrome; Pars planitis; Pemphigus; Pemphigus vulgaris; Pernicious anaemia; Perivenous encephalomyelitis; POEMS syndrome; Polyarteritis nodosa; Polymyalgia rheumatica; Polymyositis; Primary biliary cirrhosis; Primary sclerosing cholangitis; Progressive inflammatory neuropathy; Psoriasis; Psoriatic Arthritis; Pyoderma gangrenosum; Pure red cell aplasia; Rasmussen's encephalitis; Raynaud phenomenon; Relapsing polychondritis; Reiter's syndrome; Restless leg syndrome; Retroperitoneal fibrosis; Rheumatoid arthritis; Rheumatoid fever; Sarcoidosis; Schizophrenia; Schmidt syndrome; Schnitzler syndrome; Scleritis; Scleroderma; Sjögren's syndrome; Spondyloarthropathy; Sticky blood syndrome; Still's Disease; Stiff person syndrome; Subacute bacterial endocarditis (SBE); Susac's syndrome; Sweet syndrome; Sydenham Chorea; Sympathetic ophthalmia; Takayasu's arteritis; Temporal arteritis; Tolosa-Hunt syndrome; Transverse Myelitis; Ulcerative Colitis; Undifferentiated connective tissue disease; Undifferentiated spondyloarthropathy; Vasculitis; Vitiligo; Wegener's granulomatosis; Wilson's syndrome; and Wiskott-Aldrich syndrome.

In some aspects, the skin disease can include but is not limited to Acneiform eruptions; Autoinflammatory syndromes; Chronic blistering; Conditions of the mucous membranes; Conditions of the skin appendages; Conditions of the subcutaneous fat; Congenital anomalies; Connective tissue diseases (such as Abnormalities of dermal fibrous and elastic tissue); Dermal and subcutaneous growths; Dermatitis (including Atopic Dermatitis, Contact Dermatitis, Eczema, Pustular Dermatitis, and Seborrheic Dermatitis); Disturbances of pigmentation; Drug eruptions; Endocrine-related skin disease; Eosinophilic; Epidermal nevi, neoplasms, cysts; Erythemas; Genodermatoses; Infection-related skin disease; Lichenoid eruptions; Lymphoid-related skin disease; Melanocytic nevi and neoplasms (including Melanoma); Monocyte- and macrophage-related skin disease; Mucinoses; Neurocutaneous; Noninfectious immunodeficiency-related skin disease; Nutrition-related skin disease; Papulosquamous hyperkeratotic (including Palmoplantar keratodermas); Pregnancy-related skin disease; Pruritic; Psoriasis; Reactive neutrophilic; Recalcitrant palmoplantar eruptions; Resulting from errors in metabolism; Resulting from physical factors (including Ionizing radiation-induced); Urticaria and angioedema; Vascular-related skin disease.

In some aspects, the endocrine disease can include but is not limited to adrenal disorders; glucose homeostasis disorders; thyroid disorders; calcium homeostasis disorders and Metabolic bone disease; Pituitary gland disorders; and Sex hormone disorders.

In some aspects, the eye disease can include but is not limited to H00-H06 disorders of eyelid, lacrimal system and orbit; H10-H13 disorders of conjunctiva; H15-H22 disorders of sclera, cornea, iris and ciliary body; H25-H28 disorders of lens; H30-H36 disorders of choroid and retina (including H30 chorioretinal inflammation, H31 other disorders of choroid, H32 chorioretinal disorders in diseases classified elsewhere, H33 retinal detachments and breaks, H34 retinal vascular occlusions, H35 other retinal disorders, and H36 retinal disorders in diseases classified elsewhere); H40-H42 glaucoma; H43-H45 disorders of vitreous body and globe; H46-H48 disorders of optic nerve and visual pathways; H49-H52 disorders of ocular muscles, binocular movement, accommodation and refraction; H53-H54.9 visual disturbances and blindness; and H55-H59 other disorders of eye and adnexa.

In some aspects, the neurological disorder can include but is not limited to abarognosis; acquired epileptiform aphasia; acute disseminated encephalomyelitis; adrenoleukodystrophy; agenesis of the corpus callosum; agnosia; Aicardi syndrome; Alexander disease; Alien hand syndrome; allochiria; Alpers' disease; alternating hemiplegia; Alzheimer's disease; amyotrophic lateral sclerosis (see motor neuron disease); anencephaly; Angelman syndrome; angiomatosis; anoxia; aphasia; apraxia; arachnoid cysts; arachnoiditis; Arnold-Chiari malformation; arteriovenous malformation; ataxia telangiectasia; attention deficit hyperactivity disorder; auditory processing disorder; autonomic dysfunction; back pain; batten disease; Behcet's disease; Bell's palsy; benign essential blepharospasm; benign intracranial hypertension; bilateral frontoparietal polymicrogyria; Binswanger's disease; blepharospasm; Bloch-Sulzberger syndrome; brachial plexus injury; brain abscess; brain damage; brain injury; brain tumor; Brown-Sequard syndrome; canavan disease; carpal tunnel syndrome; causalgia; central pain syndrome; central pontine myelinolysis; centronuclear myopathy; cephalic disorder; cerebral aneurysm; cerebral arteriosclerosis; Cerebral atrophy; Cerebral gigantism; Cerebral palsy; Cerebral vasculitis; Cervical spinal stenosis; Charcot-Marie-Tooth disease; Chiari malformation; Chorea; Chronic fatigue syndrome; Chronic inflammatory demyelinating polyneuropathy (CIDP); Chronic pain; Coffin Lowry syndrome; Coma; Complex regional pain syndrome; Compression neuropathy; Congenital facial diplegia; Corticobasal degeneration; Cranial arteritis; Craniosynostosis; Creutzfeldt-Jakob disease; Cumulative trauma disorders; Cushing's syndrome; Cytomegalic inclusion body disease (CIBD); Cytomegalovirus Infection; Dandy-Walker syndrome; Dawson disease; De Morsier's syndrome; Dejerine-Klumpke palsy; Dejerine-Sottas disease; Delayed sleep phase syndrome; Dementia; Dermatomyositis; Developmental dyspraxia; Diabetic neuropathy; Diffuse sclerosis; Dravet syndrome; Dysautonomia; Dyscalculia; Dysgraphia; Dyslexia; Dystonia; Empty sella syndrome; Encephalitis; Encephalocele; Encephalotrigeminal angiomatosis; Encopresis; Epilepsy; Erb's palsy; Erythromelalgia; Essential tremor; Fabry's disease; Fahr's syndrome; Fainting; Familial spastic paralysis; Febrile seizures; Fisher syndrome; Friedreich's ataxia; Fibromyalgia; Gaucher's disease; Gerstmann's syndrome; Giant cell arteritis; Giant cell inclusion disease; Globoid Cell Leukodystrophy; Gray matter heterotopia; Guillain-Barré syndrome; HTLV-1 associated myelopathy; Hallervorden-Spatz disease; Head injury; Headache; Hemifacial Spasm; Hereditary Spastic Paraplegia; Heredopathia atactica polyneuritiformis; Herpes zoster oticus; Herpes zoster; Hirayama syndrome; Holoprosencephaly; Huntington's disease; Hydranencephaly; Hydrocephalus; Hypercortisolism; Hypoxia; Immune-Mediated encephalomyelitis; Inclusion body myositis; Incontinentia pigmenti; Infantile phytanic acid storage disease; Infantile Refsum disease; Infantile spasms; Inflammatory myopathy; Intracranial cyst; Intracranial hypertension; Joubert syndrome; Karak syndrome; Kearns-Sayre syndrome; Kennedy disease; Kinsbourne syndrome; Klippel Feil syndrome; Krabbe disease; Kugelberg-Welander disease; Kuru; Lafora disease; Lambert-Eaton myasthenic syndrome; Landau-Kleffner syndrome; Lateral medullary (Wallenberg) syndrome; Learning disabilities; Leigh's disease; Lennox-Gastaut syndrome; Lesch-Nyhan syndrome; Leukodystrophy; Lewy body dementia; Lissencephaly; Locked-In syndrome; Lou Gehrig's disease (See Motor Neurone Disease); Lumbar disc disease; Lumbar spinal stenosis; Lyme disease—Neurological Sequelae; Machado-Joseph disease (Spinocerebellar ataxia type 3); Macrencephaly; Macropsia; Megalencephaly; Melkersson-Rosenthal syndrome; Menieres disease; Meningitis; Menkes disease; Metachromatic leukodystrop hy; Microcephaly; Micropsia; Migraine; Miller Fisher syndrome; Mini-stroke (transient ischemic attack); Mitochondrial myopathy; Mobius syndrome; Monomelic amyotrophy; Motor Neurone Disease; Motor skills disorder; Moyamoya disease; Mucopolysaccharidoses; Multi-infarct dementia; Multifocal motor neuropathy; Multiple sclerosis; Multiple system atrophy; Muscular dystrophy; Myalgic encephalomyelitis; Myasthenia gravis; Myelinoclastic diffuse sclerosis; Myoclonic Encephalopathy of infants; Myoclonus; Myopathy; Myotubular myopathy; Myotonia congenita; Narcolepsy; Neurofibromatosis; Neuroleptic malignant syndrome; Neurological manifestations of AIDS; Neurological sequelae of lupus; Neuromyotonia; Neuronal ceroid lip ofuscinosis; Neuronal migration disorders; Niemann-Pick disease; Non 24-hour sleep-wake syndrome; Nonverbal learning disorder; O'Sullivan-McLeod syndrome; Occipital Neuralgia; Occult Spinal Dysraphism Sequence; Ohtahara syndrome; Olivopontocerebellar atrophy; Opsoclonus myoclonus syndrome; Optic neuritis; Orthostatic Hypotension; Overuse syndrome; Palinopsia; Paresthesia; Parkinson's disease; Paramyotonia Congenita; Paraneoplastic diseases; Paroxysmal attacks; Parry-Romberg syndrome; Pelizaeus-Merzbacher disease; Periodic Paralyses; Peripheral neuropathy; Persistent Vegetative State; Pervasive developmental disorders; Photic sneeze reflex; Phytanic acid storage disease; Pick's disease; Pinched nerve; Pituitary tumors; PMG; Polio; Polymicrogyria; Polymyositis; Porencephaly; Post-Polio syndrome; Postherpetic Neuralgia (PHN); Postinfectious Encephalomyelitis; Postural Hypotension; Prader-Willi syndrome; Primary Lateral Sclerosis; Prion diseases; Progressive hemifacial atrophy; Progressive multifocal leukoencephalopathy; Progressive Supranuclear Palsy; Pseudotumor cerebri; Rabies; Ramsay-Hunt syndrome (Type I and Type II); Rasmussen's encephalitis; Reflex neurovascular dystrophy; Refsum disease; Repetitive motion disorders; Repetitive stress injury; Restless legs syndrome; Retrovirus-associated myelopathy; Rett syndrome; Reye's syndrome; Rhythmic Movement Disorder; Romberg syndrome; Saint Vitus dance; Sandhoff disease; Schizophrenia; Schilder's disease; Schizencephaly; Sensory integration dysfunction; Septo-optic dysplasia; Shaken baby syndrome; Shingles; Shy-Drager syndrome; Sjögren's syndrome; Sleep apnea; Sleeping sickness; Snatiation; Sotos syndrome; Spasticity; Spina bifida; Spinal cord injury; Spinal cord tumors; Spinal muscular atrophy; Spinocerebellar ataxia; Steele-Richardson-Olszewski syndrome; Stiff-person syndrome; Stroke; Sturge-Weber syndrome; Subacute sclerosing panencephalitis; Subcortical arteriosclerotic encephalopathy; Superficial siderosis; Sydenham's chorea; Syncope; Synesthesia; Syringomyelia; Tarsal tunnel syndrome; Tardive dyskinesia; Tarlov cyst; Tay-Sachs disease; Temporal arteritis; Tetanus; Tethered spinal cord syndrome; Thomsen disease; Thoracic outlet syndrome; Tic Douloureux; Todd's paralysis; Tourette syndrome; Toxic encephalopathy; Transient ischemic attack; Transmissible spongiform encephalopathies; Transverse myelitis; Traumatic brain injury; Tremor; Trigeminal neuralgia; Tropical spastic paraparesis; Trypanosomiasis; Tuberous sclerosis; Von Hippel-Lindau disease; Viliuisk Encephalomyelitis; Wallenberg's syndrome; Werdnig-Hoffman disease; West syndrome; Whiplash; Williams syndrome; Wilson's disease; and Zellweger syndrome.

In some aspects, the cardiovascular disease can include but is not limited to aneurysm; Angina; atherosclerosis; cerebrovascular accident (stroke); cerebrovascular disease; congestive heart failure; coronary artery disease; myocardial infarction (heart attack); and peripheral vascular disease.

In some aspects, the transcriptome-wide expression profiles can be used for the determining that a subject has recovered from a disease or injury.

In some aspects, the method comprises detecting that one or more biomarkers is upregulated in a sample obtained in 1 minute, 5 minutes, 10 minutes 30 minutes, 1 hour, 2 hours, 4 hours, 6 hours, 12 hours, 18 hours, 24 hours, 2 days, 3 days, 4 days, 5 days, 6 days, 7 days, 10 days, 2 weeks, 3 weeks, 4 weeks, 1 month, 2 months, 3 months, 4 months, 5 months, 6 months, 9 months, 1 year, 2 years, 3 years, 4 years, 5 years, 6 years, 7 years, 8 years, 9 years, or 10 years before or after the onset of a sign or symptom of a disease or the diagnosis of a disease, as compared to a control sample. In some aspects, the method comprises detecting that one or more biomarkers is downregulated or upregulated in a sample obtained in the 1 minute, 5 minutes, 10 minutes 30 minutes, 1 hour, 2 hours, 4 hours, 6 hours, 12 hours, 18 hours, 24 hours, 2 days, 3 days, 4 days, 5 days, 6 days, 7 days, 10 days, 2 weeks, 3 weeks, 4 weeks, 1 month, 2 months, 3 months, 4 months, 5 months, 6 months, 9 months, 1 year, 2 years, 3 years, 4 years, 5 years, 6 years, 7 years, 8 years, 9 years, or 10 years before or after the onset of a sign or symptom of a disease or the diagnosis of a disease, as compared to a control sample. In some aspects, the control sample is the level of the one or more biomarkers at baseline, as measured in a sample obtained prior to the onset of a sign or symptom of a disease or injury. For example, in some aspects, the methods can comprise detecting the upregulation by determining that the change in expression of the one or more biomarkers in the subject a sample obtained in the 1 minute, 5 minutes, 10 minutes 30 minutes, 1 hour, 2 hours, 4 hours, 6 hours, 12 hours, 18 hours, 24 hours, 2 days, 3 days, 4 days, 5 days, 6 days, 7 days, 10 days, 2 weeks, 3 weeks, 4 weeks, 1 month, 2 months, 3 months, 4 months, 5 months, 6 months, 9 months, 1 year, 2 years, 3 years, 4 years, 5 years, 6 years, 7 years, 8 years, 9 years, or 10 years before or after the onset of a sign or symptom of a disease or the diagnosis of a disease compared to baseline is greater than the change in expression of the one or more biomarkers in a control subject or population that does not have a sign or symptom of the disease or has not been diagnosed with the disease. In some aspects, the methods can comprise detecting that one or more biomarkers is downregulated in a subject a sample obtained in the 1 minute, 5 minutes, 10 minutes 30 minutes, 1 hour, 2 hours, 4 hours, 6 hours, 12 hours, 18 hours, 24 hours, 2 days, 3 days, 4 days, 5 days, 6 days, 7 days, 10 days, 2 weeks, 3 weeks, 4 weeks, 1 month, 2 months, 3 months, 4 months, 5 months, 6 months, 9 months, 1 year, 2 years, 3 years, 4 years, 5 years, 6 years, 7 years, 8 years, 9 years, or 10 years before or after the onset of a sign or symptom of a disease or the diagnosis of a disease, as compared to a control. For example, in some aspects, the methods can comprise detecting the downregulation by determining that the change in expression of the one or more biomarkers in the subject from the a sample obtained in the 1 minute, 5 minutes, 10 minutes 30 minutes, 1 hour, 2 hours, 4 hours, 6 hours, 12 hours, 18 hours, 24 hours, 2 days, 3 days, 4 days, 5 days, 6 days, 7 days, 10 days, 2 weeks, 3 weeks, 4 weeks, 1 month, 2 months, 3 months, 4 months, 5 months, 6 months, 9 months, 1 year, 2 years, 3 years, 4 years, 5 years, 6 years, 7 years, 8 years, 9 years, or 10 years before or after the onset of a sign or symptom of a disease or the diagnosis of a disease compared to baseline is less than the change in expression of the one or more biomarkers in a control subject or population that has not experienced or does not have a sign or symptom of a disease or injury or has not been diagnosed with a disease.

In some aspects, the methods can comprise detecting one or more markers in a biological sample of the subject. In some aspects, the level of one or more of markers in the biological test sample of the subject is compared with the level of the biomarker in a comparator. Non-limiting examples of comparators include, but are not limited to, a negative control, a positive control, standard control, standard value, an expected normal background value of the subject, a historical normal background value of the subject, a reference standard, a reference level, an expected normal background value of a population that the subject is a member of, or a historical normal background value of a population that the subject is a member of. In some aspects, the comparator can be a level of the one or more biomarker in a sample obtained from the subject prior to the onset of a sign or symptom of a disease or injury. In some aspects, the comparator can be a level of the one or more biomarker in an earlier obtained sample from the subject after the onset of a sign or symptom of a disease or injury but before the collection of the test sample.

In some aspects, the methods can include monitoring the progression of a disease or injury in a subject by assessing the level of one or more of the markers in a biological sample of the subject.

In some aspects, the subject can a human subject, and can be of any race, sex and age. In some aspects, the subject can be healthy or have a disease or injury.

In some aspects, the information obtained from the methods described herein can be used alone, or in combination with other information (e.g., disease status, disease history, vital signs, blood chemistry, neurological score, etc.) from the subject or from the biological sample obtained from the subject.

In some aspects of the methods disclosed herein, the level of one or more markers can be determined to be increased when the level of one or more of the markers is increased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator.

In some aspects of the methods disclosed herein, the level of one or more markers can be determined to be decreased when the level of one or more of the markers is decreased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator.

In some aspects, a biological sample from a subject can be assessed for the level of one or more of the markers in the biological sample obtained from the patient. The level of one or more of the markers in the biological sample can be determined by assessing the amount of polypeptide of one or more of the biomarkers in the biological sample, the amount of mRNA of one or more of the biomarkers in the biological sample, the amount of enzymatic activity of one or more of the biomarkers in the biological sample, or a combination thereof.

Disclosed herein are compositions and methods relating to biomarkers that can be used for making a transcriptome-wide expression profile or for the diagnoses of disease in a subject. The biomarkers can be used to screen, diagnose, monitor the onset, monitor the progression, and assess the recovery of a disease. The biomarkers can be used to establish and evaluate treatment plans. In some aspects, the transcriptome-wide expression profiles can be used to identify biomarkers. In some aspects, once the biomarkers are identified, the biomarkers can be used to make gene signature that then can be used screen, diagnose, monitor the onset, monitor the progression, and assess the recovery of a disease.

Disclosed herein are compositions and methods for making a transcriptome-wide expression profile. Disclosed herein are compositions and methods that can be used for diagnosing and providing a prognosis for a disease. In some aspects, the methods can comprise examining relevant biomarkers and their expression. In some aspects, biomarker expression includes transcription into messenger RNA (mRNA) and translation into protein. In aspects, the methods can comprise determining if the expression levels of the relevant biomarkers are differentially expressed as compared to a control. In some aspects, the control can be the level of the relevant biomarkers in a subject not having a disease, a population not having a disease, a subject who has not recovered from a disease, a population that has not recovered from a disease, a subject that has recovered from a disease, a population that has recovered from a disease, and a control sample of the subject being diagnosed where the control sample is obtained prior to a sign or symptom of the disease. In some aspects, the methods can comprise determining if the expression levels of the relevant biomarkers in a sample obtained from the subject are differentially expressed as compared to the expression levels of the relevant biomarkers in an earlier obtained sample from the same subject, which was obtained at an earlier time point following the onset of a risk factor or a sign or symptom of the disease. In some aspects, the methods can comprise detecting the expression levels of the relevant biomarkers across a plurality of samples obtained from the same subject overtime, thereby providing a time course of biomarker expression.

Accordingly, in some aspects, methods for diagnosing a disease are provided. The methods comprise a) providing a biological sample from the subject; b) analyzing the biological sample with an assay that specifically detects at least one biomarker of the invention in the biological sample; c) comparing the level of the at least one biomarker in the sample with the level in a control sample or earlier obtained biological sample, wherein a statistically significant difference between the level of the at least one biomarker in the sample with the level in a control sample or earlier obtained biological sample is indicative of brain injury. In some embodiments, the methods further comprise the step of d) effectuating a treatment regimen based thereon. In some aspects, the method comprises analyzing the change in gene expression over a defined time interval in the subject, and comparing detected change in gene expression with the change in gene expression observed in a control subject.

In some aspects, methods for determining the prognosis or treatment regimen of a disease are provided. The methods can comprise a) providing a biological sample from the subject; b) analyzing the biological sample with an assay that specifically detects at least one biomarker in the biological sample; c) comparing the level of the at least one biomarker in the sample with the level in a control sample or earlier obtained biological sample, wherein a statistically significant difference between the level of the at least one biomarker in the sample with the level in a control sample or earlier obtained biological sample is indicative of the prognosis or treatment regimen of the disease. In some aspects, the method comprises analyzing the change in gene expression over a defined time interval in the subject, and comparing detected change in gene expression with the change in gene expression observed in a control subject.

In some aspects, the biomarker types can comprise mRNA biomarkers. In some aspects, the mRNA can be detected by at least one of mass spectroscopy, PCR microarray, thermal sequencing, capillary array sequencing, solid phase sequencing, and the like.

In some aspects, the biomarker types can comprise polypeptide biomarkers. In some aspects, the polypeptide can detected by at least one of ELISA, Western blot, flow cytometry, immunofluorescence, immunohistochemistry, mass spectroscopy, and the like.

Detecting a Biomarker

Disclosed herein are methods of detecting one or more mRNA biomarkers, polypeptide biomarkers, or a combination thereof in a biological sample. Biomarkers generally can be measured and detected through a variety of assays, methods and detection systems known to one of skill in the art.

Examples of methods include but are not limited to immunoassays, microarray, PCR, RT-PCR, refractive index spectroscopy (RI), ultra-violet spectroscopy (UV), fluorescence analysis, electrochemical analysis, radiochemical analysis, near-infrared spectroscopy (near-IR), infrared (IR) spectroscopy, nuclear magnetic resonance spectroscopy (NMR), light scattering analysis (LS), mass spectrometry, pyrolysis mass spectrometry, nephelometry, dispersive Raman spectroscopy, gas chromatography, liquid chromatography, gas chromatography combined with mass spectrometry, liquid chromatography combined with mass spectrometry, matrix-assisted laser desorption ionization-time of flight (MALDI-TOF) combined with mass spectrometry, ion spray spectroscopy combined with mass spectrometry, capillary electrophoresis, colorimetry and surface plasmon resonance (such as according to systems provided by Biacore Life Sciences). In some aspects, biomarkers can be measured using the above-mentioned detection methods, or other methods known to the skilled artisan. Other biomarkers can be similarly detected using reagents that are specifically designed or tailored to detect them.

Different types of biomarkers and their measurements can be combined in the compositions and methods described herein. In some aspects, the protein form of the biomarkers can be measured. In some aspects, the nucleic acid form of the biomarkers can be measured. In some aspects, the nucleic acid form can be mRNA. In some aspects, measurements of protein biomarkers can be used in conjunction with measurements of nucleic acid biomarkers.

Disclosed herein are methods of measuring polypeptide levels in a biological sample obtained from a subject include, but are not limited to, an immunochromatography assay, an immunodot assay, a Luminex assay, an ELISA assay, an ELISPOT assay, a protein microarray assay, a ligand-receptor binding assay, displacement of a ligand from a receptor assay, displacement of a ligand from a shared receptor assay, an immunostaining assay, a Western blot assay, a mass spectrophotometry assay, a radioimmunoassay (RIA), a radioimmunodiffusion assay, a liquid chromatography-tandem mass spectrometry assay, an ouchterlony immunodiffusion assay, reverse phase protein microarray, a rocket immunoelectrophoresis assay, an immunohistostaining assay, an immunoprecipitation assay, a complement fixation assay, FACS, an enzyme-substrate binding assay, an enzymatic assay, an enzymatic assay employing a detectable molecule, such as a chromophore, fluorophore, or radioactive substrate, a substrate binding assay employing such a substrate, a substrate displacement assay employing such a substrate, and a protein chip assay.

Methods for detecting a nucleic acid (e.g., mRNA), such as RT-PCR, real time PCR, microarray, branch DNA, NASBA and others, are well known in the art. Using sequence information provided by the database entries for the biomarker sequences, expression of the biomarker sequences can be detected (if present) and measured using techniques known to one of ordinary skill in the art. For example, sequences in sequence database entries or sequences disclosed herein can be used to construct probes for detecting biomarker RNA sequences in, e.g., Northern blot hybridization analyses or methods which specifically, and, quantitatively amplify specific nucleic acid sequences. As another example, the sequences can be used to construct primers for specifically amplifying the biomarker sequences in, e.g., amplification-based detection methods such as reverse-transcription based polymerase chain reaction (RT-PCR). When alterations in gene expression are associated with gene amplification, deletion, polymorphisms and mutations, sequence comparisons in test and reference populations can be made by comparing relative amounts of the examined DNA sequences in the test and reference cell populations. In addition to Northern blot and RT-PCR, RNA can also be measured using, for example, other target amplification methods (e.g., TMA, SDA, NASBA), signal amplification methods (e.g., bDNA), nuclease protection assays, in situ hybridization and the like.

In some aspects, quantitative hybridization methods, such as Southern analysis, Northern analysis, or in situ hybridizations, can be used. A “nucleic acid probe,” as used herein, can be a DNA probe or an RNA probe. The probe can be, for example, a gene, a gene fragment (e.g., one or more exons), a vector comprising the gene, a probe or primer, etc. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate target mRNA or cDNA. The hybridization sample can be maintained under conditions which are sufficient to allow specific hybridization of the nucleic acid probe to mRNA or cDNA. Specific hybridization can be performed under high stringency conditions or moderate stringency conditions, as appropriate. In some aspects, the hybridization conditions for specific hybridization can be high stringency. Specific hybridization, if present, is then detected using standard methods. If specific hybridization occurs between the nucleic acid probe having a mRNA or cDNA in the test sample, the level of the mRNA or cDNA in the sample can be assessed. More than one nucleic acid probe can also be used concurrently in this method. Specific hybridization of any one of the nucleic acid probes can be indicative of the presence of the mRNA or cDNA of interest, as described herein.

Alternatively, a peptide nucleic acid (PNA) probe can be used instead of a nucleic acid probe in the quantitative hybridization methods described herein. PNA is a DNA mimic having a peptide-like, inorganic backbone, such as N-(2-aminoethyl)glycine units, with an organic base (A, G, C, T or U) attached to the glycine nitrogen via a methylene carbonyl linker. The PNA probe can be designed to specifically hybridize to a target nucleic acid sequence. Hybridization of the PNA probe to a nucleic acid sequence can be used to determine the level of the target nucleic acid in the biological sample.

In some aspects, arrays of oligonucleotide probes that are complementary to target nucleic acid sequences in the biological sample obtained from a subject can be used to determine the level of one or more biomarkers in the biological sample obtained from a subject. The array of oligonucleotide probes can be used to determine the level of one or more biomarkers alone, or the level of the one or more biomarkers in relation to the level of one or more other nucleic acids in the biological sample. Oligonucleotide arrays typically comprise a plurality of different oligonucleotide probes that are coupled to a surface of a substrate in different known locations. These oligonucleotide arrays, also known as “Genechips,” have been generally described in the art, for example, U.S. Pat. No. 5,143,854 and PCT patent publication Nos. WO 90/15070 and 92/10092. These arrays can generally be produced using mechanical synthesis methods or light directed synthesis methods which incorporate a combination of photolithographic methods and solid phase oligonucleotide synthesis methods.

After an oligonucleotide array is prepared, a nucleic acid of interest can be hybridized with the array and its level can be quantified. Hybridization and quantification are generally carried out by methods described herein. In brief, a target nucleic acid sequence can be amplified by well-known amplification techniques, e.g., PCR. Typically, this involves the use of primer sequences that are complementary to the target nucleic acid. Asymmetric PCR techniques can also be used. Amplified target, generally incorporating a label, can then be hybridized with the array under appropriate conditions. Upon completion of hybridization and washing of the array, the array can be scanned to determine the quantity of hybridized nucleic acid. The hybridization data obtained from the scan can typically be in the form of fluorescence intensities as a function of quantity, or relative quantity, of the target nucleic acid in the biological sample. The target nucleic acid can be hybridized to the array in combination with one or more comparator controls (e.g., positive control, negative control, quantity control, etc.) to improve quantification of the target nucleic acid in the sample.

The probes and primers as described herein can be labeled directly or indirectly with a radioactive or nonradioactive compound, by methods known to those skilled in the art, in order to obtain a detectable and/or quantifiable signal; the labeling of the primers or of the probes is carried out with radioactive elements or with nonradioactive molecules. Among the radioactive isotopes used, mention may be made of ₃₂P, ₃₃P, ₃₅S or ₃H. The nonradioactive entities can selected from ligands such as biotin, avidin, streptavidin or digoxigenin, haptenes, dyes, and luminescent agents such as radioluminescent, chemiluminescent, bioluminescent, fluorescent or phosphorescent agents.

Nucleic acids can be obtained from the cells using known techniques. Nucleic acid herein refers to RNA, including mRNA, and DNA, including cDNA. The nucleic acid can be double-stranded or single-stranded (i.e., a sense or an antisense single strand) and can be complementary to a nucleic acid encoding a polypeptide. The nucleic acid content can also be an RNA or DNA extraction performed on a biological sample, including a biological fluid and fresh or fixed tissue sample.

Many methods are known in the art for the detection and quantification of specific nucleic acid sequences and new methods are continually reported. A great majority of the known specific nucleic acid detection and quantification methods utilize nucleic acid probes in specific hybridization reactions. In some aspects, the detection of hybridization to the duplex form can be a Southern blot technique. In the Southern blot technique, a nucleic acid sample can be separated in an agarose gel based on size (molecular weight) and affixed to a membrane, denatured, and exposed to (admixed with) the labeled nucleic acid probe under hybridizing conditions. If the labeled nucleic acid probe forms a hybrid with the nucleic acid on the blot, the label can be bound to the membrane.

In the Southern blot, the nucleic acid probe can be labeled with a tag. In some aspects, the tag can be a radioactive isotope, a fluorescent dye or the other well-known materials. Another type of process for the specific detection of nucleic acids in a biological sample are hybridization methods. In some aspects, a nucleic acid probe of at least 10 nucleotides, at least 15 nucleotides, or at least 25 nucleotides, having a sequence complementary to a nucleic acid of interest can be hybridized in a sample, subjected to depolymerizing conditions, and the sample can be treated with an ATP/luciferase system, which will luminesce if the nucleic sequence is present. In quantitative Southern blotting, the level of the nucleic acid of interest can be compared with the level of a second nucleic acid of interest, and/or to one or more comparator control nucleic acids (e.g., positive control, negative control, quantity control, etc.).

In some aspects, the method useful for the detection and quantification of nucleic acid can be the polymerase chain reaction (PCR). The PCR process is known in the art. To briefly summarize PCR, nucleic acid primers, complementary to opposite strands of a nucleic acid amplification target sequence, are permitted to anneal to the denatured sample. A DNA polymerase (typically heat stable) extends the DNA duplex from the hybridized primer. The process can be repeated to amplify the nucleic acid target. If the nucleic acid primers do not hybridize to the sample, then there is no corresponding amplified PCR product. In this case, the PCR primer acts as a hybridization probe.

In PCR, the nucleic acid probe can be labeled with a tag as described herein. In some aspects, the detection of the duplex can be done using at least one primer directed to the nucleic acid of interest. In some aspects of PCR, the detection of the hybridized duplex can comprise electrophoretic gel separation followed by dye-based visualization.

Typical hybridization and washing stringency conditions depend in part on the size (i.e., number of nucleotides in length) of the oligonucleotide probe, the base composition and monovalent and divalent cation concentrations.

In some aspects, the process for determining the quantitative and qualitative profile of the nucleic acid of interest as described herein can be characterized in that the amplifications are real-time amplifications performed using a labeled probe or a labeled hydrolysis-probe, capable of specifically hybridizing in stringent conditions with a segment of the nucleic acid of interest. The labeled probe can be capable of emitting a detectable signal every time each amplification cycle occurs, allowing the signal obtained for each cycle to be measured.

The real-time amplification, such as real-time PCR, is known in the art, and the various known techniques can be employed in the best way for the implementation of the present process. These techniques can be performed using various categories of probes, such as hydrolysis probes, hybridization adjacent probes, or molecular beacons. The techniques employing hydrolysis probes or molecular beacons are based on the use of a fluorescence quencher/reporter system, and the hybridization adjacent probes are based on the use of fluorescence acceptor/donor molecules.

Hydrolysis probes with a fluorescence quencher/reporter system are commercially available. Many fluorescent dyes can be employed, such as FAM dyes (6-carboxy-fluorescein), or any other dye phosphoramidite reagents.

Among the stringent conditions applied for any one of the hydrolysis-probes described herein is the Tm, which is in the range of about 65° C. to 75° C. In some aspects, the Tm for any one of the hydrolysis-probes described herein can be in the range of about 67° C. to about 70° C. In some aspects, the Tm applied for any one of the hydrolysis-probes described herein can be about 67° C.

In some aspects, the methods described herein can include a primer that is complementary to a nucleic acid of interest, and more particularly the primer includes 12 or more contiguous nucleotides substantially complementary to the nucleic acid of interest. In some aspects, a primer that can be used in the methods described herein can include a nucleotide sequence sufficiently complementary to hybridize to a nucleic acid sequence of about 12 to 25 nucleotides.

In some aspects, the primer differs by no more than 1, 2, or 3 nucleotides from the target flanking nucleotide sequence. In some aspects, the length of the primer can vary in length, for example, from about 15 to 28 nucleotides in length (e.g., 16, 17, 18, 19, 20, 21, 22, 23, 24, 25, 26, or 27 nucleotides in length).

The concentration of a biomarker in a sample can be determined by any suitable assay. A suitable assay can include one or more of the following methods, an enzyme assay, an immunoassay, mass spectrometry, chromatography, electrophoresis or an antibody microarray, or any combination thereof. Thus, as would be understood by one skilled in the art, the system and methods disclosed herein may include any method known in the art to detect a biomarker in a sample.

Also disclosed herein are methods for a multiplex analysis platform. In some aspects, the method comprises an analytical method for multiplexing analytical measurements of markers.

As used herein, the term “expression,” when used in the context of determining or detecting the expression or expression level of one or more biomarkers, can refer to determining or detecting transcription of the biomarker (e.g., gene; i.e., determining mRNA levels) and/or determining or detecting translation of the biomarker (e.g., determining or detecting the protein produced). To determine the expression level of a biomarker means to determine whether or not a biomarker is expressed, and if expressed, to what relative degree.

The expression level of one or more biomarkers disclosed herein can be determined directly (e.g., immunoassays, mass spectrometry) or indirectly (e.g., determining the mRNA expression of a protein or peptide). Examples of mass spectrometry include ionization sources such as EI, CI, MALDI, ESI, and analysis such as Quad, ion trap, TOF, FT or combinations thereof, spectrometry, isotope ratio mass spectrometry (IRMS), thermal ionization mass spectrometry (TIMS), spark source mass spectrometry, Multiple Reaction Monitoring (MRM) or SRM. Any of these techniques can be carried out in combination with prefractionation or enrichment methods. Examples of immunoassays include immunoblots, Western blots, Enzyme linked Immunosorbant Assay (ELISA), Enzyme immunoassay (EIA), radioimmune assay.

Immunoassay methods use antibodies for detection and determination of levels of an antigen are known in the art. The antibody can be immobilized on a solid support such as a stick, plate, bead, microbead or array.

Expression levels of one or more of the biomarkers described herein can be also be determined indirectly by determining the mRNA expression for the one or more biomarkers in a biological sample. RNA expression methods include but are not limited to extraction of cellular mRNA and Northern blotting using labeled probes that hybridize to transcripts encoding all or part of a gene, amplification of mRNA using gene-specific primers, polymerase chain reaction (PCR), and reverse transcriptase-polymerase chain reaction (RT-PCR), followed by quantitative detection of the gene product by a variety of methods; extraction of RNA from cells, followed by labeling, and then used to probe cDNA or oligonucleotides encoding the gene, in situ hybridization; and detection of a reporter gene.

Methods to measure protein expression levels include but are not limited to Western blot, immunoblot, ELISA, radioimmunoassay, immunoprecipitation, surface plasmon resonance, chemiluminescence, fluorescent polarization, phosphorescence, immunohistochemical analysis, microcytometry, microarray, microscopy, fluorescence activated cell sorting (FACS), and flow cytometry. The method can also include specific protein property-based assays based including but not limited to enzymatic activity or interaction with other protein partners. Binding assays can also be used, and are well known in the art. For instance, a BIAcore machine can be used to determine the binding constant of a complex between two proteins. Other suitable assays for determining or detecting the binding of one protein to another include, immunoassays, such as ELISA and radioimmunoassays. Determining binding by monitoring the change in the spectroscopic can be used or optical properties of the proteins can be determined via fluorescence, UV absorption, circular dichroism, or nuclear magnetic resonance (NMR).

As used herein, the term “comparator” can be used interchangeably with “reference,” “reference expression,” “reference sample,” “reference value,” “control,” “control sample” and the like, when used in the context of a sample or expression level of one or more biomarkers, one or more genes or proteins refers to a reference standard wherein the reference is expressed at a constant level in a sample, and is unaffected by the experimental conditions, and is indicative of the level in a sample of a predetermined disease status. The reference value can be a predetermined standard value or a range of predetermined standard values, representing no disease or illness, or a predetermined type or severity of a disease or illness.

Reference expression can be the level of the one or more biomarkers or genes described herein in a reference sample from a subject, or a pool of subjects, not suffering from a disease or from a predetermined severity or type of disease. In some aspects, the reference value can be the level of one or more biomarkers or genes described herein in the sample from a subject, or subjects, wherein the subject or subjects is considered healthy and not suffering from a particular disease.

In some aspects, the expression level of one or more biomarkers or genes can be compared. By comparing the expression level for one or more biomarkers obtained from a subject with the reference expression level, it is possible to determine a subject's susceptibility to a disease.

Determining the expression level of one or more biomarkers or genes can include determining whether the biomarker or gene is upregulated or increased as compared to a control or reference sample, downregulated or decreased compared to a control or reference sample, or unchanged compared to a control or reference sample. As used herein, the terms, “upregulated” and “increased expression level” or “increased level of expression” refers to a sequence corresponding to one or more biomarkers or genes that is expressed wherein the measure of the quantity of the sequence exhibits an increased level of expression when compared to a reference sample (e.g., from a “diseased control” or a “normal” control). For example, the terms, “upregulated” and “increased expression level” or “increased level of expression” refers to a sequence corresponding to one or more biomarkers or genes that is expressed wherein the measure of the quantity of the sequence exhibits an increased level of expression of the one or more biomarkers (e.g., protein(s) and/or mRNA) when compared to the expression of the same mRNA(s) from a reference sample (e.g., from a “diseased control” or a “normal” control). An “increased expression level” refers to an increase in expression of at least 1%, 2%, 3%, 4%, 5%, 6%, 7%, 8%, 9%, 10% or more, for example, 20%, 30%, 40%, or 50%, 60%, 70%, 80%, 90% or more, or greater than 1-fold, up to 2-fold, 3-fold, 4-fold, 5-fold, 10-fold, 50-fold, 100-fold or more. As used herein, the terms “downregulated,” “decreased level of expression,” or “decreased expression level” refers to a sequence corresponding to one or more biomarkers or genes that is expressed wherein the measure of the quantity of the sequence exhibits a decreased level of expression when compared to a reference sample (e.g., from a “diseased control” or a “normal” control). For example, the terms “downregulated,” “decreased level of expression,” or “decreased expression level” refers to a sequence corresponding to one or more biomarkers or genes that is expressed wherein the measure of the quantity of the sequence exhibits a decreased level of expression of one or more biomarkers (e.g., protein(s) and/or mRNA) when compared to the expression of the same mRNA(s) from a reference sample (e.g., from a “diseased control” or a “normal” control). A “decreased level of expression: refers to a decrease in expression of at least 1%, 2%, 3%, 4%, 5%, 6%, 7%, 8%, 9%, 10% or more, for example, 20%, 30%, 40%, or 50%, 60%, 70%, 80%, 90% or more, or greater than 1-fold, up to 2-fold, 3-fold, 4-fold, 5-fold, 10-fold, 50-fold, 100-fold or more.

In some aspects, the methods can include determining whether a subject has an increased susceptibility to a disease. As described herein, samples from a subject can be compared with reference samples to determine the expression ratio to determine whether a subject has an increased susceptibility to a disease. The reference samples can be from subjects having “normal” levels of one or more biomarkers or genes. Suitable statistical and other analysis can be carried out to confirm a change (e.g., an increase or a higher level of expression) in one or more biomarkers when compared with a reference sample, wherein a ratio of the sample expression level of one or more biomarkers to the reference expression level of one or more biomarkers indicates higher expression level of one or more biomarkers in the sample. In some aspects, the ratio of the sample expression level of two or more, three or more, four or more, five or more, or six or more biomarkers to the reference expression level of two or more, three or more, four or more, five or more, or six or more biomarkers indicates higher expression level of two or more, three or more, four or more, five or more, or six or more biomarkers in the sample, indicating that the subject has an increased susceptibility to a disease.

A higher or increased expression level of one or more biomarkers when compared to the reference expression level the one or more biomarkers can indicate an increased susceptibility to a disease. Signature pattern(s) of increased (higher) or decreased (lower) sample expression levels of one or more biomarkers when compared to the reference expression levels of one or more biomarkers can be observed and indicate the susceptibility (e.g., higher or lower) of a disease in a subject.

The expression level of one or more biomarkers or genes described herein can be a measure of one or more biomarkers or genes, for example, per unit weight or volume. In some aspects, the expression level can be a ratio (e.g., the amount of one or more biomarkers or genes in a sample relative to the amount of the one or more biomarkers of a reference value).

In some aspects, samples from a subject can be compared with reference samples to determine the percent change to determine whether a subject has an increased susceptibility to a disease. In other words, the expression level can be expressed as a percent. For example, the percent change in the expression levels of one or more biomarkers or genes, wherein the expression level of one (or two, three, four, five or six) or more of the biomarkers is increased (or is higher) by 10%, 15%, 20%, 25%, 30%, 35%, 40%, 45%, 50%, 55%, 60%, 65%, 70%, 75%, 80%, 85%, 90%, 95%, or 100% when compared to the reference expression level the biomarker, indicating an increased susceptibility to a disease. Alternatively, the percent change in the expression levels of one or more biomarkers or genes can be decreased (or lower) by 10%, 15%, 20%, 25%, 30%, 35%, 40%, 45%, 50%, 55%, 60%, 65%, 70%, 75%, 80%, 85%, 90%, 95%, or 100% when compared to a reference expression level.

In some aspects, an increase or decrease or some combination thereof in the expression level of biomarkers genes or proteins can indicate an increased susceptibility for a disease or a diagnosis of a disease in a subject. In some aspects, a signature pattern of increased or decreased expression levels of one or more of the biomarkers, genes or proteins disclosed herein is indicative.

In some aspects, the methods disclosed herein can further include a method of prevention of disease morbidity and/or mortality. For example, the method comprises providing to a subject, further testing (which can include testing for the disease), such as, for example, a routine physical examination, wherein an increased susceptibility to a disease has been diagnosed. The method can further include the administration of therapy to prevent the disease from developing or spreading, thereby reducing disease morbidity and/or mortality.

Kits

Disclosed herein are kits useful in the methods described herein. In some aspects, the kits can comprise various combinations of components useful in any of the methods described herein, including for example, materials for quantitatively analyzing a biomarker (e.g., polypeptide and/or nucleic acid), materials for assessing the activity of a biomarker (e.g., polypeptide and/or nucleic acid), and instructional material. For example, in some aspects, the kit can comprise components useful for the quantification of a desired nucleic acid in a biological sample. In some aspects, the kit can comprise components useful for the quantification of a desired polypeptide in a biological sample. In some aspects, the kit can comprise components useful for the assessment of the activity (e.g., enzymatic activity, substrate binding activity, etc.) of a desired polypeptide in a biological sample.

In some aspects, the kit can comprise the components of an assay for monitoring the effectiveness of a treatment administered to a subject in need thereof, containing instructional material and the components for determining whether the level of a biomarker in a biological sample obtained from the subject is modulated during or after administration of the treatment. In some aspects, to determine whether the level of a biomarker is modulated in a biological sample obtained from the subject, the level of the biomarker can be compared with the level of at least one comparator control contained in the kit, such as a positive control, a negative control, a historical control, a historical norm, or the level of another reference molecule in the biological sample. In some aspects, the ratio of the biomarker and a reference molecule can be determined to aid in the monitoring of the treatment.

In an aspect, kits are provided for measuring the RNA (e.g., a RNA product) of one or more biomarkers disclosed herein. The kits can comprise materials and reagents that can be used for measuring the expression of the RNA of one or more biomarkers. Examples of suitable kits include RT-PCR or microarray. These kits can include the reagents needed to carry out the measurements of the RNA expression levels. Alternatively, the kits can further comprise additional materials and reagents. For example, the kits can comprise materials and reagents required to measure RNA expression levels of any number of genes up to 1, 2, 3, 4, 5, 10, or more genes that are not biomarkers disclosed herein.

Methods of Treatment

Disclosed herein are methods for diagnosing a disease or injury, assessing disease or injury severity, and assessing recovery from the disease or injury in a subject who has or has not experienced a sign or symptom of a disease or injury by generating a transcriptome-wide expression profile. In some aspects, the methods for making a transcriptome-wide expression profile of a biological sample can be used for diagnosing a disease or injury, assessing disease or injury severity, and assessing recovery from the disease or injury in a subject who has or has not experienced a sign or symptom of a disease or injury by generating a transcriptome-wide expression profile. In some aspects, the methods for making a transcriptome-wide expression profile of a biological sample can comprise: a) measuring the expression levels of one or more genes present in a first biological sample, wherein said measuring comprises RNA sequencing; b) determining the expression levels of the genes from the measured expression levels obtained in step a); c) combining the results of steps a) and b) to produce a transcriptome-wide expression profile; and d) providing the transcriptome-wide expression profile as a data set, wherein the first biological sample consists of isolated platelets. In some aspects, the first and second biological samples can be obtained from the same subject or from different subjects. In some aspects, steps a)-d) can be repeated on each biological sample. In some aspects, the transcriptome-wide expression profile of the subjects can be compared. In some aspects, the first and second biological samples can be obtained from the same subject at a first time point and a second time point. In some aspects, the first and second biological samples can be obtained from different subjects, further comprising obtaining additional biological samples from the subjects, wherein the additional biological samples are obtained at different time points. In some aspects, the first and second time points can be different time points. In some aspects, the transcriptome-wide expression profile from the first time point can be compared to the transcriptome-wide expression profile from the second time point. In some aspects, said methods further comprise repeating the steps thereof until a validated transcriptome-wide expression profile can be identified. In some aspects, the expression levels can be measured on a device selected from the group consisting of a microarray, a bead array, a liquid array, and a nucleic-acid sequence. In some aspects, the methods can further comprising treating a subject diagnosed with disease or injury or one or more symptoms of a disease or injury.

Disclosed herein are methods of treating comprising administering a disease-modulating drug to a subject. The drug can be a therapeutic or prophylactic used in subjects diagnosed or identified with a disease or at risk of having the disease. In some aspects, the modifying therapy refers to altering the duration, frequency or intensity of therapy, for example, altering dosage levels.

In some aspects, effecting a therapy can comprise causing a subject to or communicating to a subject the need to undergo a therapy. In some aspects, the therapy can be surgery.

In some aspects, the measurement of biomarker levels allows for the course of treatment of a disease to be monitored. The effectiveness of a treatment regimen for a disease can be monitored by detecting one or more biomarkers in an effective amount from samples obtained from a subject over time and comparing the amount of biomarkers detected. For example, a first sample can be obtained prior to the subject receiving treatment and one or more subsequent samples can be taken after or during treatment of the subject. Changes in biomarker levels across the samples may provide an indication as to the effectiveness of the therapy.

In some aspects, to identify therapeutics or drugs that are appropriate for a specific subject, a test sample from the subject can also be exposed to a therapeutic agent or a drug, and the level of one or more biomarkers can be determined. Biomarker levels can be compared to a sample derived from the subject before and after treatment or exposure to a therapeutic agent or a drug, or can be compared to samples derived from one or more subjects who have shown improvements relative to a disease as a result of such treatment or exposure. Thus, disclosed herein are methods of assessing the efficacy of a therapy comprising taking a first measurement of a biomarker panel in a first sample from the subject; effecting the therapy with respect to the subject; taking a second measurement of the biomarker panel in a second sample from the subject and comparing the first and second measurements to assess the efficacy of the therapy.

Additionally, therapeutic agents suitable for administration to a particular subject can be identified by detecting one or more biomarkers in an effective amount from a sample obtained from a subject and exposing the subject-derived sample to a test compound that determines the amount of the biomarker(s) in the subject-derived sample. Accordingly, treatments or therapeutic regimens for use in subjects having a disease (or one or more signs or symptoms of a disease) can be selected based on the amounts of biomarkers in samples obtained from the subjects and compared to a reference value. Two or more treatments or therapeutic regimens can be evaluated in parallel to determine which treatment or therapeutic regimen would be the most efficacious for use in a subject to delay onset, or slow progression of a disease. In some aspects, a recommendation can be made on whether to initiate or continue treatment of a disease.

In some aspects, effecting a therapy can comprise administering a disease-modulating drug to the subject. The subject can be treated with one or more drugs until altered levels of the measured biomarkers return to a baseline value measured in a population not having a disease, having a less severe form of the disease, or showing improvements in disease biomarkers as a result of treatment with a drug. In some aspects, the subject can be treated with one or more drugs until altered levels of the measured biomarkers return to a baseline value measured in a pre-symptomatic sample obtained from the subject. Additionally, improvements related to a changed level of a biomarker or clinical parameter can be the result of treatment with a disease-modulating drug.

Any drug or combination of drugs disclosed herein may be administered to a subject to treat a disease. The drugs herein can be formulated in any number of ways, often according to various known formulations in the art or as disclosed or referenced herein.

In some aspects, any drug or combination of drugs disclosed herein is not administered to a subject to treat a disease. In some aspects, the practitioner may refrain from administering the drug or combination of drugs, may recommend that the subject not be administered the drug or combination of drugs or may prevent the subject from being administered the drug or combination of drugs.

In some aspects, one or more additional drugs can be optionally administered in addition to those that are recommended or have been administered. An additional drug will typically not be any drug that is not recommended or that should be avoided.

### EXAMPLES

### Example 1: Longitudinal RNA-Seq Analysis of the Repeatability of Gene Express and Splicing in Human Platelets Identifies a SELP Splice QTL

Longitudinal studies are required to distinguish within versus between-individual variation, and repeatability of gene expression. They are positioned to decipher genetic signal from environmental noise, with potential application to gene variant and expression studies. However, longitudinal analyses of gene expression in healthy individuals—especially with regards to alternative splicing—are lacking for most primary cell types, including platelets.

A method to assess repeatability of gene expression and splicing in platelets and use repeatability to identify novel platelet eQTLs and sQTLs was performed.

The transcriptome of platelets isolated repeatedly up to 4 years from healthy individuals was sequenced. Within and between-individual variation and repeatability of platelet RNA-expression and exon skipping, a readily measured alternative splicing event was examined. The results show that platelet gene expression is generally stable between and within individuals over time—with the exception of a subset of genes enriched for the inflammation gene ontology. The results also show an enrichment among repeatable genes for associations with heritable traits, including known and novel platelet eQTLs. Several exon skipping events were also highly repeatable, suggesting heritable patterns of splicing in platelets. One of the most repeatable was exon 14 skipping of SELP. Accordingly, rs6128 was identified as a platelet sQTL and defined an rs6128-dependent association between SELP exon 14 skipping and race. In vitro experiments demonstrate that this single nucleotide variant directly affects exon 14 skipping, and changes the ratio of transmembrane versus soluble P-selectin protein production.

The platelet transcriptome is generally stable over 4-years. The findings demonstrated the use of repeatability of gene expression and splicing to identify novel platelet eQTLs and sQTLs. rs6128 is a platelet sQTL that alters SELP exon 14 skipping and soluble versus transmembrane P-selectin protein production.

In this Example, longitudinal RNA-seq analysis was used to examine within (intra-) and between (inter-) individual variability of the human platelet transcriptome. Repeatability of gene abundance and exon skipping, a readily measured alternative splicing event, was examined. Repeatability demonstrated retrospectively its use to decipher heritable signal from environmental noise, and identify eQTL genes. Repeatability also prospectively was used to prioritize eQTL and sQTL gene candidates for novel platelet eQTL and sQTL discovery.

Material and Methods. Human Subjects and Platelet Isolation.

Subjects were healthy and without active medical conditions. No subjects had undergone surgery for the past 4 months. Any illness required resolution of symptoms for at least 7 days prior to sampling. Cohort 2 subjects were prospectively recruited. Cohort 1 subjects were part of a clinical study where they were previously exposed to aspirin, however, no subjects were on aspirin for at least 4 weeks prior to each blood sampling. Blood was drawn by venipuncture into citrate tubes (cohort 1) or acid-citrate-dextrose (cohort 2) and sample processing initiated within 30 minutes of phlebotomy. Platelets were isolated via magnetic leukocyte depletion using CD45 microbeads (Miltenyi) (Rowley J W, et al. Blood. 2011; 118:e101-e111; Bray P F, et al. BMC Genomics. 2013; 14:1; and Voora D, Cyr D, et al. J Am Coll Cardiol. 2013; 62:1267-76).

RNA isolation and sequencing. RNA was isolated using phenol-chloroform extraction (cohort 1) (Rowley J W, et al. Blood. 2011; 118:e101-e111) or DirectZol kit (cohort 2, Zymogen). Sequencing libraries for cohorts 1 and 2 were barcoded and prepared using kits: KAPA Stranded mRNA-Seq Kit (Roche #KK8421) and TruSeq unstranded v2 with poly(A) selection (Illumina #RS-122) respectively. Libraries were sequenced 50 cycles, single end, on Illumina HiSeq 4000 (cohort 1) or Illumina HiSeq 2000 (cohort 2) to a depth of ˜20-40 million mapped reads per sample. Fastq files have been deposited in the NIH Sequence Read Archive PRJNA531691.

RNA-seq analysis. For analysis of expression variation, reads were aligned to GRCh38/hg38 using Novoalign (Novocraft) (Rowley J W, et al. Blood. 2011; 118:e101-e111). Reads were assigned to flattened Ensembl gene annotations using the USeq analysis package (Nix D A, et al. BMC Bioinformatics. 2010; 11:455). Read counts were normalized separately for each cohort using the DESeq2 analysis package (Love M I, et al. Genome Biology. 2014; 15:550). Non-coding RNAs were selected according to Ensembl transcript biotype. Heatmaps, clustering (complete linkage), density plots, boxplots, and scatterplots, were generated in R (R Development Core Team. R: A Language and Environment for Statistical Computing. 2018; Available from: http://www.r-project.org). Read distribution plots were generated using integrative genomics viewer (IGV) (Thorvaldsdóttir H, et al. Brief Bioinform. 2013; 14:178-92). Gene ontology enrichment was analyzed using DAVID (Huang D W, et al. Nat Protoc. 2009; 4:44-57).

RNA isolation and sequencing. Following isolation, fresh platelet pellets were suspended in 1 mL of Trizol and frozen at −80 c until RNA isolation. RNA from cohort 1 was isolated using phenol-chloroform extraction, isopropanol precipitation in the presence of glycogen, and 75% ethanol washes. Samples were DNAse treated (Invitrogen #AM1907), and RNA re-purified using ammonium acetate/isopropanol precipitation (Rowley J W, et al. Blood. 2011; 118:e101-e111). RNA from cohort 2 was isolated and DNAse treated using DirectZol kit and columns (Zymogen).

Analysis of individual transcript variation. RNA-seq data has a strong mean-variance relationship. The DESeq2 regularized log transformation (RLD) (Love M I, et al. Genome Biology. 2014; 15:550) was applied to counts which preferentially shrinks the overall variance among low abundant transcripts (while retaining outliers), thus allowing a more straightforward comparison of transcript variation across the expression levels. Lowest abundant transcripts were also arbitrarily removed. Within-individual variation was calculated as the standard deviation of the samples from the same individual. Total variation was calculated as the standard deviation of samples across the individuals in each cohort, thus within-individual variation is a sub-component of total individual variation. Sources of variance were quantified with a linear mixed model using the R package variance Partition (Hoffman G E, et al. BMC Bioinformatics. 2016; 17:483). Repeatability was calculated with the formula σ_{b}²/(σ_{w}²+σ_{b}²) in the R package ‘heritability’(Kruijer W, et al. Genetics. 2015; 199:379-98), including correction for sex in repeatability calculations for eQTL enrichment analysis and gene prioritization for eQTL and sQTL discovery.

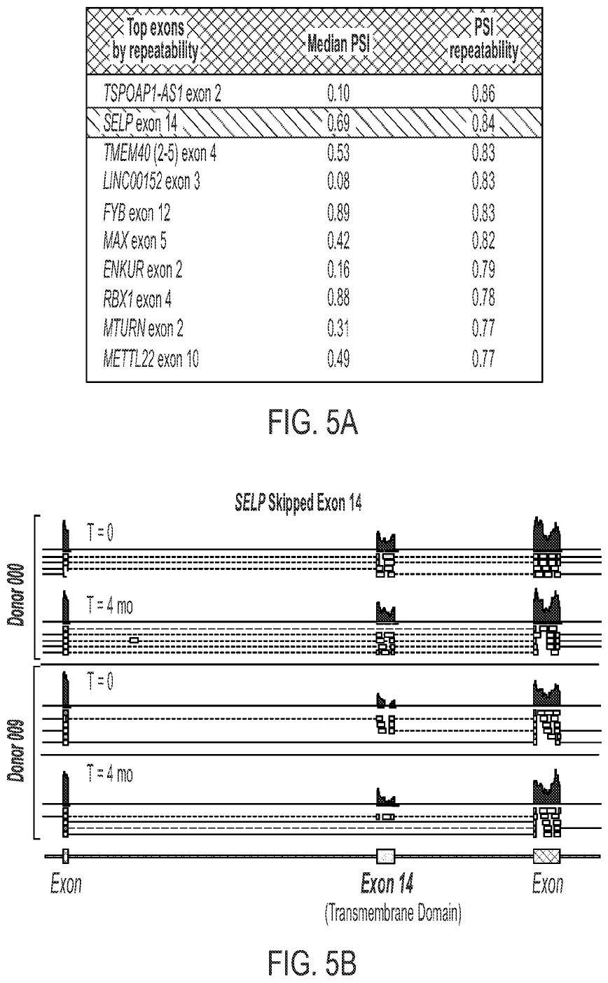

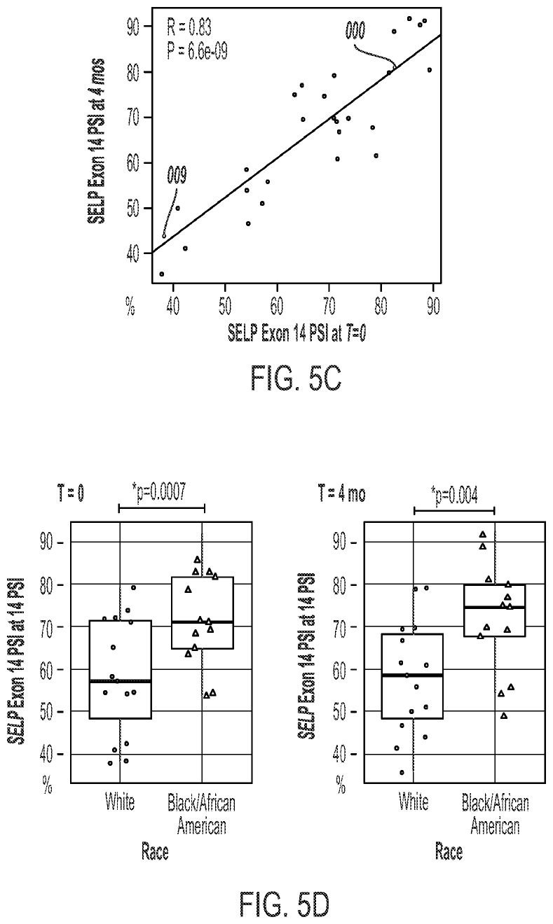

Exon skipping analysis. Exon skipping events were identified from triads of Exon/Exon and Exon/Intron junctions with >5 reads per junction and the calculated percent spliced in (PSI; see FIG. 5D)>0.05 and <0.95 in >30% of samples. Junctions were excluded where the flanking Exon/Intron junction pairs varied by more than 10 fold in >70% of samples. By this strategy, 245 exons (from 194 different transcripts) were identified that were skipped in a significant fraction of the transcripts in some samples.

SELP mini-gene. The complete open reading frame for SELP transcript ENST00000263686.10 with a c-terminal DYK tag, and introns 13 and 14 that flank exon 14, were cloned into PCDNA-CMV and pCDH-MSCV-GFP vectors. A single nucleotide change C->T was made at rs6128. 293T cells (HEK 293T/17, ATCC CRL-11268) were maintained according to ATCC recommendations and used between passages 10-20. 293T cells were transfected with lipofectamine 2000. 24 hours after transfection, SELP RNA splicing was analyzed by PCR at cycles 25 and 30 using primers flanking exon 14 (5′-gtcaactaccgtgccaacct (SEQ ID NO: 1); 5′-taaggactcgggtcaaatgc (SEQ ID NO: 2). For flow cytometry experiments, cells were either co-transfected with a GFP plasmid (PCDNA-CMV) or GFP was contained within the same backbone as SELP (PCDH-MSCV). Surface expression of P-selectin was assessed by staining with Psel.K02.3 APC antibody (ThermoFisher #17-0626-82) and analyzed by flow cytometry on a CytoFLEX analyzer (Beckman Coulter). MFI of P-selectin was normalized to GFP expression as assessed on live/transfected cells gated according to forward/side scatter (live) and FL-1 (GFP+) intensity. Soluble P-selectin was measured using a Quantikine ELISA kit (R&D Systems #DPSE00). For western blots, cells were lysed with RIPA, lysates denatured and reduced, proteins separated using an 10% SDS-PAGE, transferred onto a PVDF membrane, and blotted for tagged P-selectin with anti-DYK antibody (Cell Signaling Tech. #2368S), followed by anti-rabbit HRP secondary antibody (Rockland #18-8816-33) and chemiluminescent detection (ThermoFisher #34580).

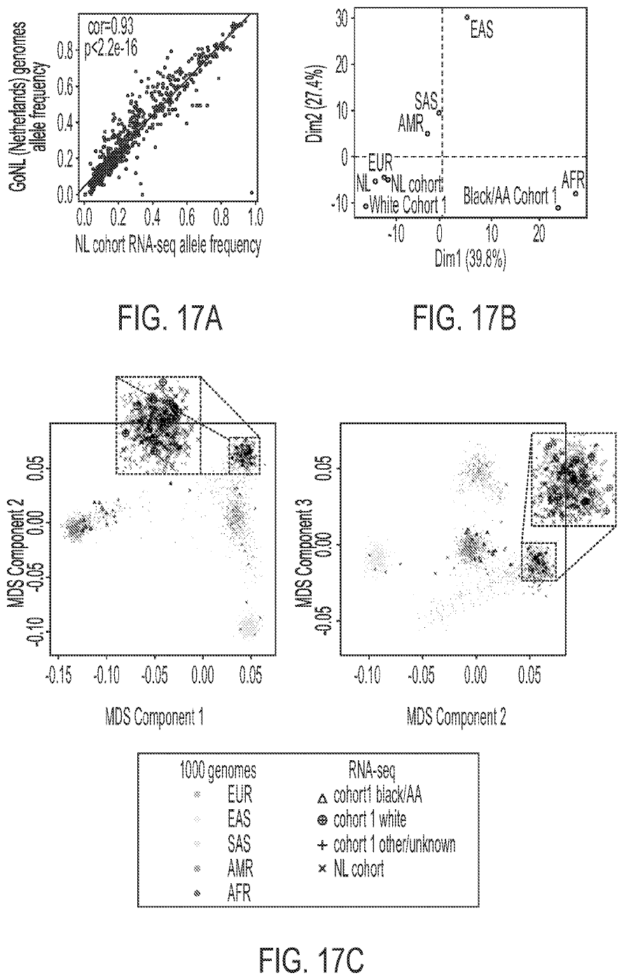

Novel platelet eQTL and sQTL analysis. RNA-seq fastq files for 234 previously published samples (Best et. al. (Best M G, et. al. Cancer Cell. 2015; 28:666-676; and Best M G, et. al. Cancer Cell. 2017; 32:238-252), Netherlands cohort; hereafter referred to as NL cohort) were retrieved from NCBI short read archive PRJNA353588 (Best M G, et. al. Cancer Cell. 2017; 32:238-252). These, and fastq files for cohort 1, were aligned with STAR (Dobin A, et al. Bioinformatics. 2013; 29:15-21) to human reference genome (build HG38) in a splice-aware manner, and variants were called and filtered using the workflow built from the Genome Analysis Toolkit (GATK) (McKenna A, et al. Genome Res. 2010; 20:1297-1303) best practices for variant calling on RNA-seq. Variants tested for eQTLs were limited to within 2 kb of the genes not identified as eQTLs by the PRAX1 (Simon L M, et al. Am J Hum Genet. 2016; 98:883-97) dataset, and with repeatability >0.9 (238 genes) in the cohort 1 dataset. For comparison, the equivalent number of genes with lowest repeatability were also included. Combined filtering resulted in 641 variants across 181 genes that were tested for gene abundance-variant association. The RNA-seq allele frequencies of these variants was comparable to DNA allele frequencies reported by the Genome of the Netherlands project (GoNL (Genome of the Netherlands Consortium, Francioli L C, Menelaou A, et. al. Whole-genome sequence variation, population structure and demographic history of the Dutch population. Nat Genet. 2014; 46:818-825)) and 1000 genomes (Gibbs R A, et al. Nature. 2015; 526:68-74) (FIG. 17A), and clustered according to the expected populations by PCA analysis of allele frequencies (FIG. 17B). RNA-seq and expected allele frequencies for significant variants. To assess population stratification, transcriptome wide variants were called. Multidimensional scaling (MDS) was implemented in Plink 1.9 (Purcell S, et al. Am J Hum Genet. 2007; 81:559-75; and Chang C C, et al. Gigascience. 2015; 4:7) on 1994 variants (filtered and pruned: FS>30.0; QD<2.0; clusterSize=3, clusterWindowSize=35; --geno 0.2; --hwe 10e-6; --maf 0.01; --indep-pairwise 50 5 0.2) co-identified from RNA-seq in cohorts 1, the NL cohort, and from 1000 genomes. Visual inspection of MDS plots indicated that individual RNA-seq samples clustered according to expected genetic ancestry, and that population structure was captured within the first 4 MDS components (FIG. 17C). Variance Stabilizing Transformation (VST) in the package DESeq2 (Love M I, et al. Genome Biology. 2014; 15:550) was used to normalize gene expression. Gene-variant association was tested in the R package SNPassoc (Gonzalez J R, et al. Bioinformatics. 2007; 23:654-655) using an additive model of variant-allele dosage (0,1,2), while controlling for the covariates sex, age, and population structure (Purcell S, et al. Am J Hum Genet. 2007; 81:559-75; and Chang C C, et al. Gigascience. 2015; 4:7) (see FIG. 17 and Methods for details). Benjamini and Hochberg FDR correction for multiple testing (641 gene-variant tests) is reported. However, a conservative significance threshold of p<1e-6 was used to filter novel eQTLs as if genome wide analysis had been performed (Simon L M, et al. Am J Hum Genet. 2016; 98:883-97). Allelic imbalance of each significant eQTL was evaluated with the Wilcoxon rank sum test on the proportion of reference variant reads to total reads in each heterozygote individual. After significance testing, eQTLs were further limited to those reported in dbSNP to minimize the possibility of RNA-specific (i.e. RNA-editing) calls. At this threshold, 27 variants across 11 new platelet eQTL genes were identified. These remained significant after further controlling for the first 5 latent variables estimated from surrogate variable analysis (SVA) (Leek J T, Storey J D. PLoS Genet. 2007; 3:e161) with explicit adjustment for sex and age. The 27 significant gene-variant associations were then tested in Cohort 1 using the strategy described herein for the NL cohort, with sex, age, and race included as covariates. However, because of the small sample size, a codominant model was allowed when 1 of the 3 genotypes required for additive model testing was missing. The results of cohort 1 eQTL analysis included a comparatively small sample size, thus, significance in cohort 1 was not expected and was not considered as criteria for novel platelet eQTL selection.

A generalized linear model was used for logistic regression analysis in R to test the association of SELP exon 14 splicing and self-reported race or rs6128 variant-allele dosage. For this, SELP exon 14 inclusion counts versus total inclusion+exclusion counts were used as the binomial response variable. Where specified, models controlled for potential covariates including race or population structure, rs6128 genotype, sex, and age.

There are recognized strengths and limitations of using RNA-seq to infer genetic variants (Piskol R, Ramaswami G, Li J B. Am J Hum Genet. 2013; 93:641-51). RNA variant calls are of different quality than genome calls. They are limited to expressed regions which has precluded fine mapping of causal eQTLs. Where there is extreme allelic imbalance, genetic variants can also be missed. As with any eQTL analysis, false positives are also possible, such as from confounding LD and genetic substructure not accounted for by large-scale population stratification. Because of the low density of variants in RNA-seq data, fine-scale structure analysis is not possible. With such limitations, additional observations are helpful in interpreting the results: the allele frequencies of RNA-seq calls for the significant variants aligned with expected allele frequencies (with the exception of rs879095052 in HBG1), 22/27 of the eQTLs (for 7/11 of the eGenes) have been reported in other tissues (The Genotype-Tissue Expression (GTEx) Project), and variants for 8/11 eGenes demonstrated allelic imbalance that was directionally consistent with the eQTL effect on expression.

Statistics. Significance for multimodality was calculated according to Hartigan's dip test statistic. Wilcoxon test with adjustment for multiple comparisons was used to test for similarity in distributions of between and within sample correlations. Kolmogorov-Smirnov test was used to test for enrichment in the rank of genes associated with genetic/heritable traits. Gene Set Enrichment Analysis (GSEA) (Subramanian A, et al. Proc Natl Acad Sci US A. 2005; 102:15545-50) pre-ranked was used to evaluate enrichment among genes tested for the presence of significant eQTLs as identified in the PRAX1 cohort. For this, the subset of genes tested by PRAX1 were included. The association between eQTL presence and different repeatability thresholds was estimated with odds ratio (odds at each threshold compared to no threshold) and significance evaluated with Chi-square test of independence. Two sided T-tests and correlation tests for significance (alpha=0.05) were performed in R (R Development Core Team. R: A Language and Environment for Statistical Computing. 2018; Available from: www.r-project.org) using functions cor.test and t.test, which sets a lower p value limit of 2.2e-16.

Results. To assess within and between individual variation of the platelet transcriptome, RNA-seq analysis of leuko-depleted platelets isolated longitudinally from two independent cohorts of healthy individuals was used. For cohort 1, platelets from 31 individuals were analyzed at an initial visit (TO) and 4 months later. For cohort 2, platelets from 7 individuals at TO and then longitudinally over 4 years were analyzed. The characteristics of the two cohorts are detailed in Table 1.

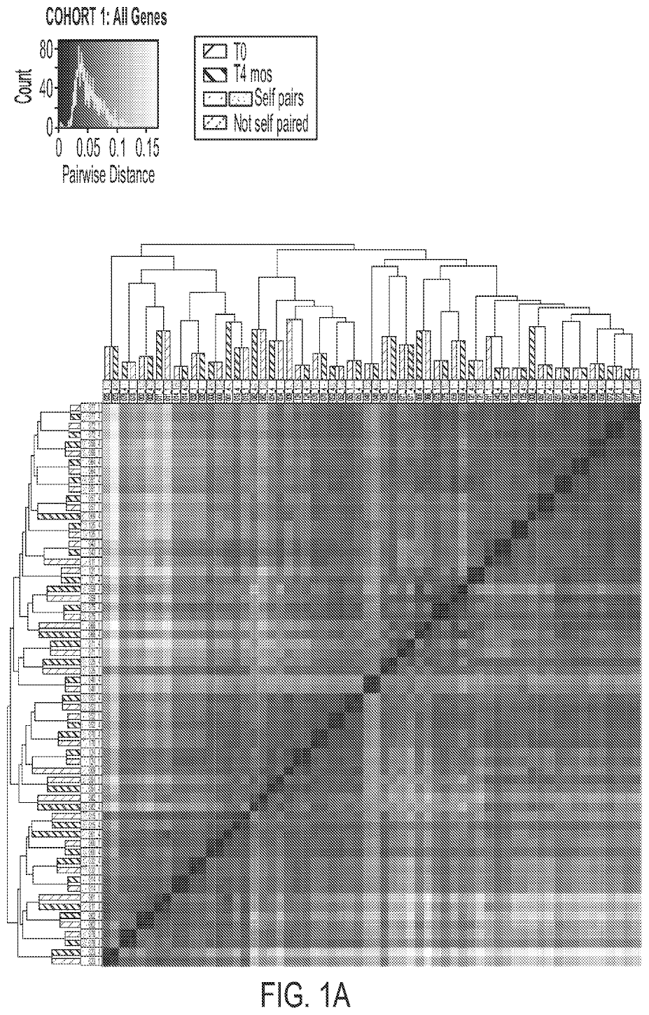

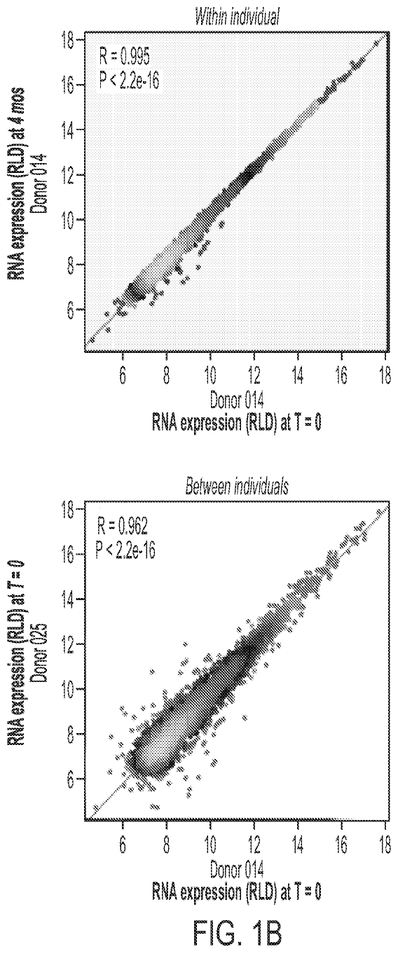

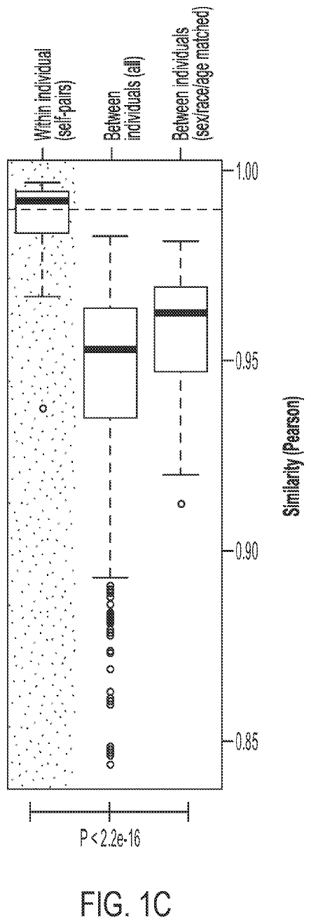

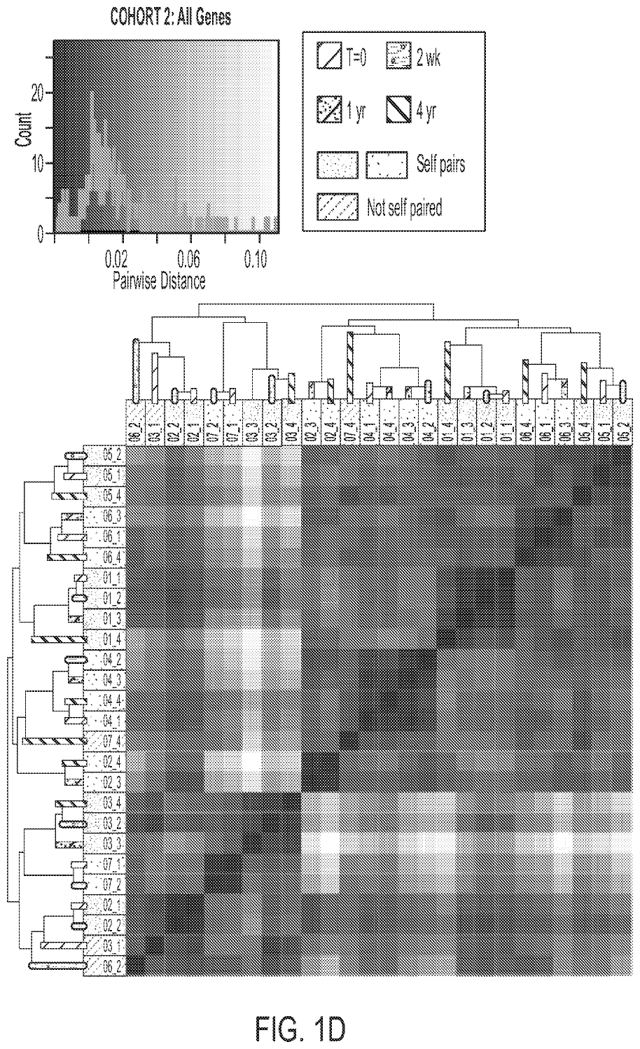

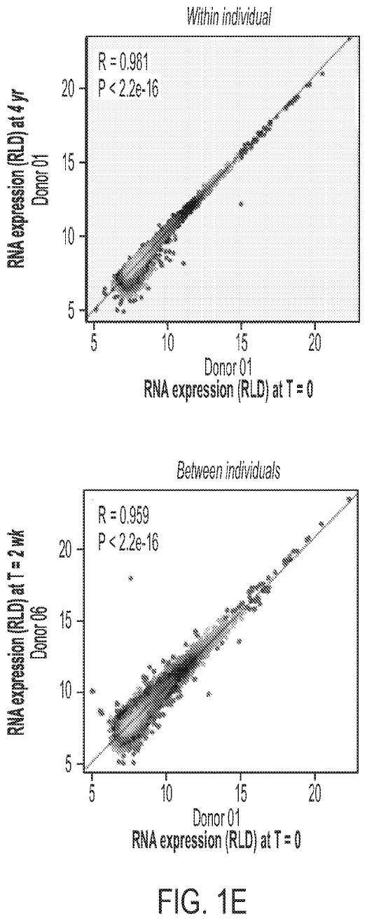

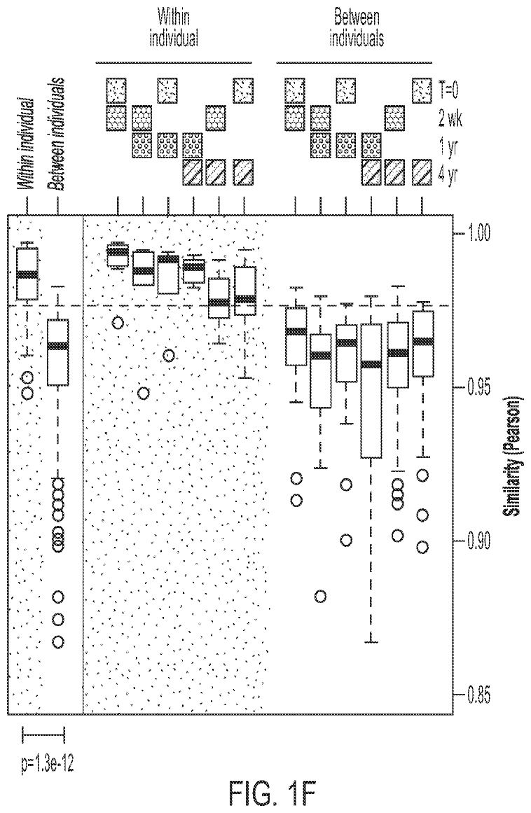

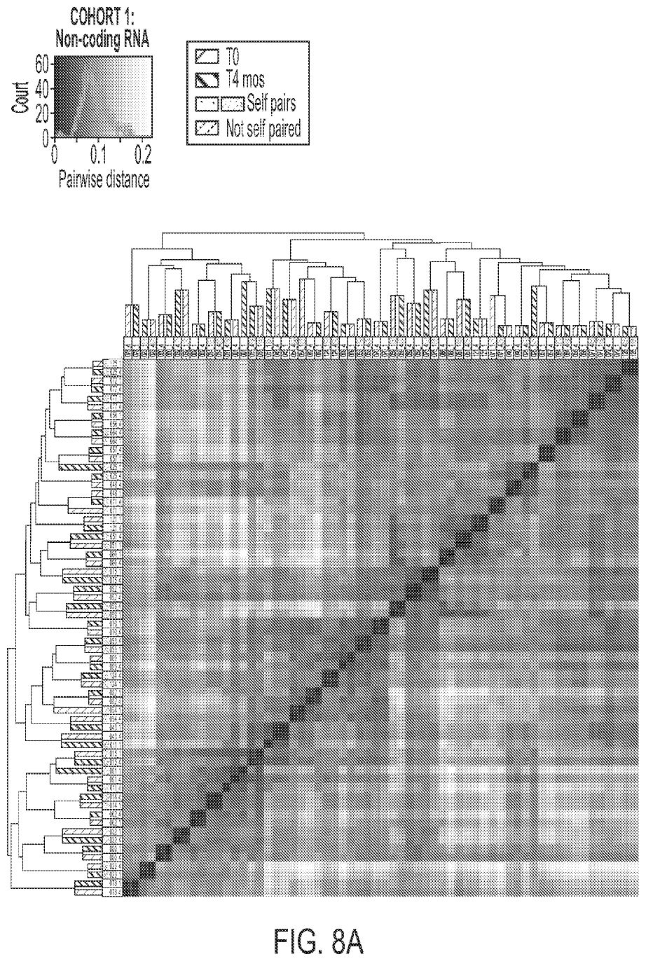

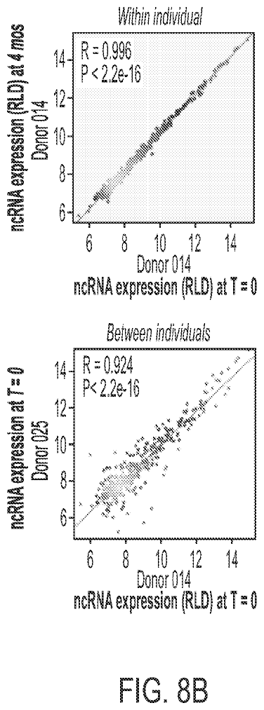

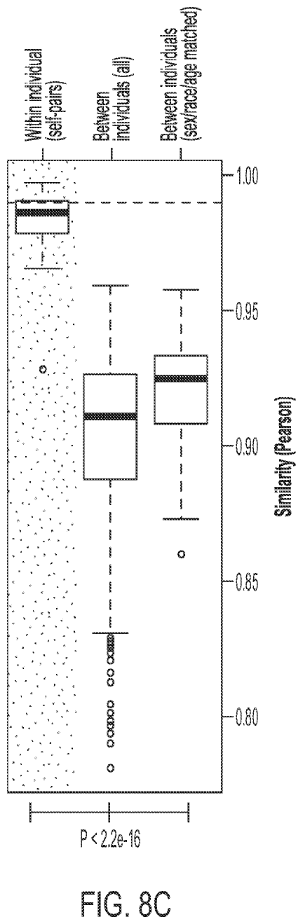

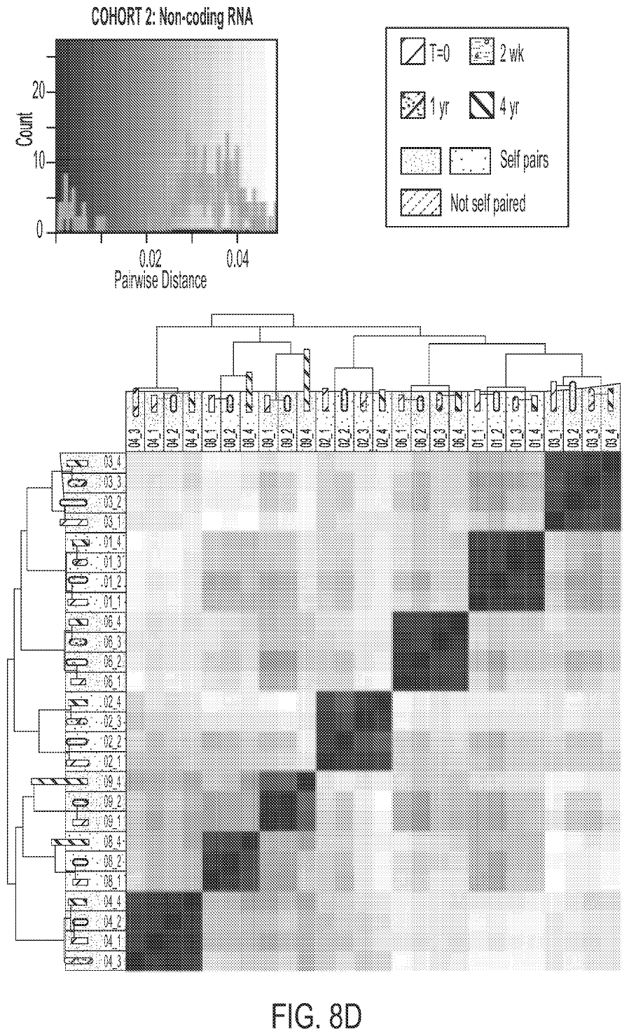

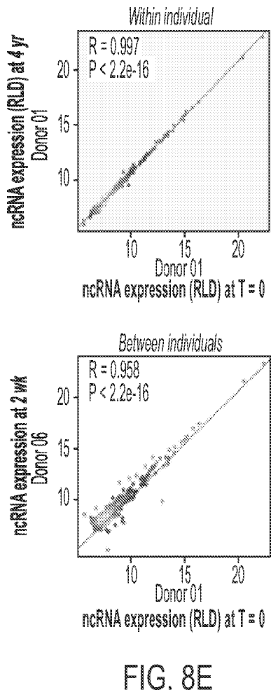

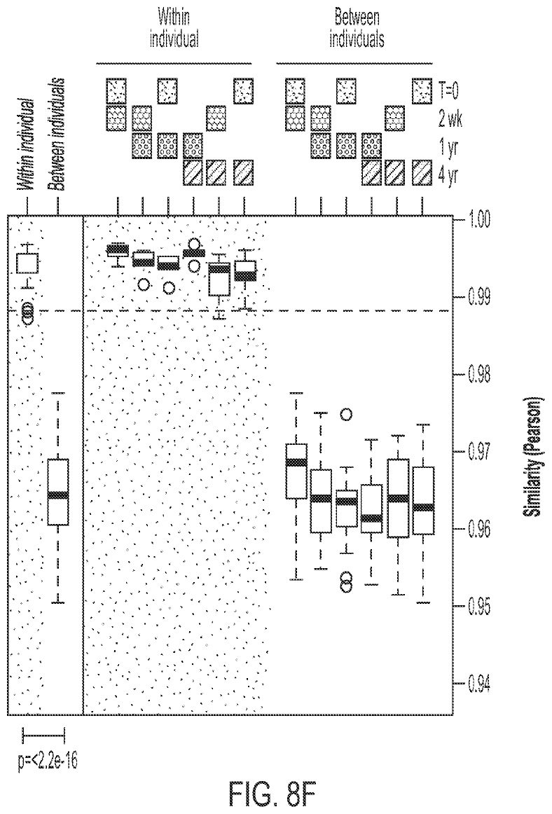

Platelets contain a stable within-individual gene expression signature. Unsupervised clustering analysis of the pair-wise distances within and between individual transcriptomes in cohort 1 indicated a robust within-individual (self) RNA expression signature (FIG. 1A), with most self-pairs clustering as nearest neighbor pairs. As depicted in FIGS. 1B-C, the mean within-individual correlation of platelet transcriptomes isolated 4 months apart was very high (r mean±sd=0.987±0.012). Between-individual correlations of the global platelet transcriptome were also high (0.947±0.024), but significantly lower (p<2.2e-16) than within-individual correlations. Grouping samples by race, age, or sex partly corrected the difference (FIG. 1C). Within individual clustering of non-coding RNAs was also robust (FIG. 8A), with a mean within-individual correlation of 0.984±0.013. The mean between individual correlation for non-protein coding transcripts (0.905±0.031, FIG. 8B-C) was significantly weaker compared to protein coding transcripts (p<2.2e-16), which is consistent with previous cross-sectional studies. Raw and normalized counts for each transcript was determined in cohort 1.

Unsupervised clustering of total RNA transcriptomes in cohort 2 resulted in robust self-clustering and suggested minimal transcriptional drift over 4 years (FIG. 1D). Within-individual correlations for samples isolated 4 years apart remained comparable to within-individual correlations for samples isolated 2 weeks apart, and significantly higher than between-individual correlations regardless of time-point (FIG. 1E-F). As shown in FIG. 8D, the within-self non-coding RNA signature was also robust, and uniquely identified individuals at the time points over the 4 years without exception—a reflection of the significantly higher within-self correlations in non-coding RNA expression compared to those between-individuals (FIG. 8E-F). Raw and normalized counts for each transcript in cohort 2 were determined.

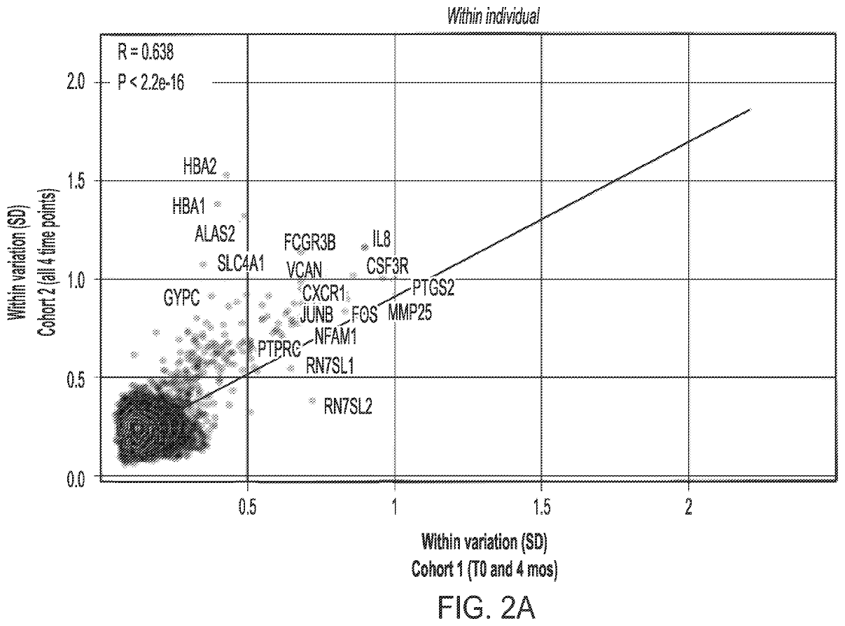

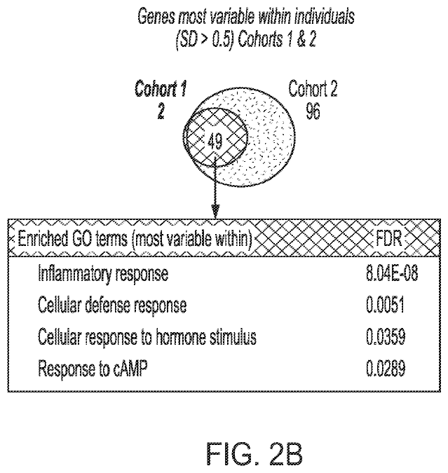

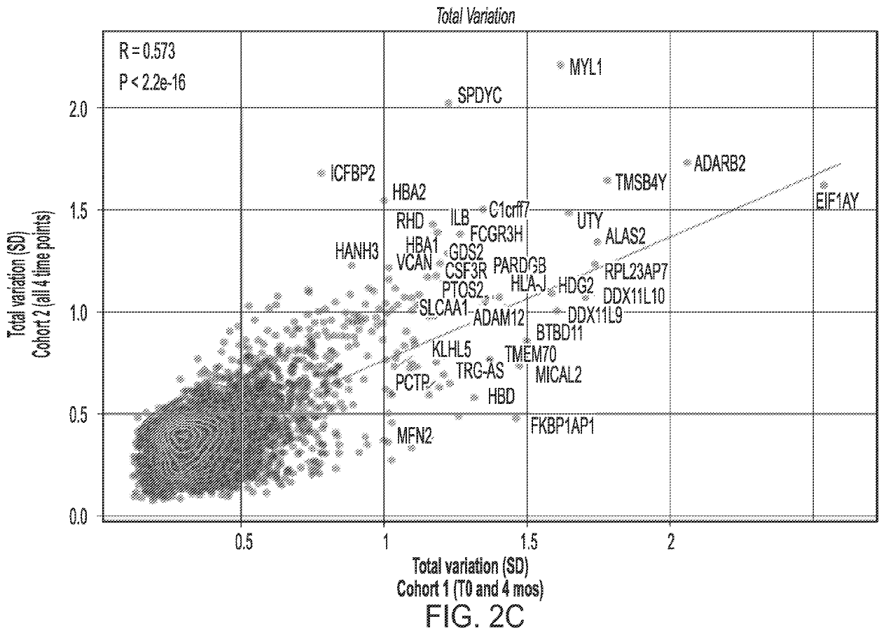

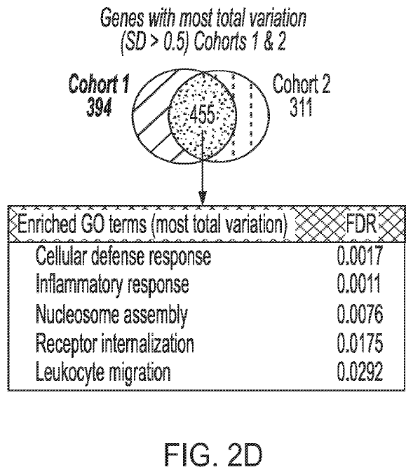

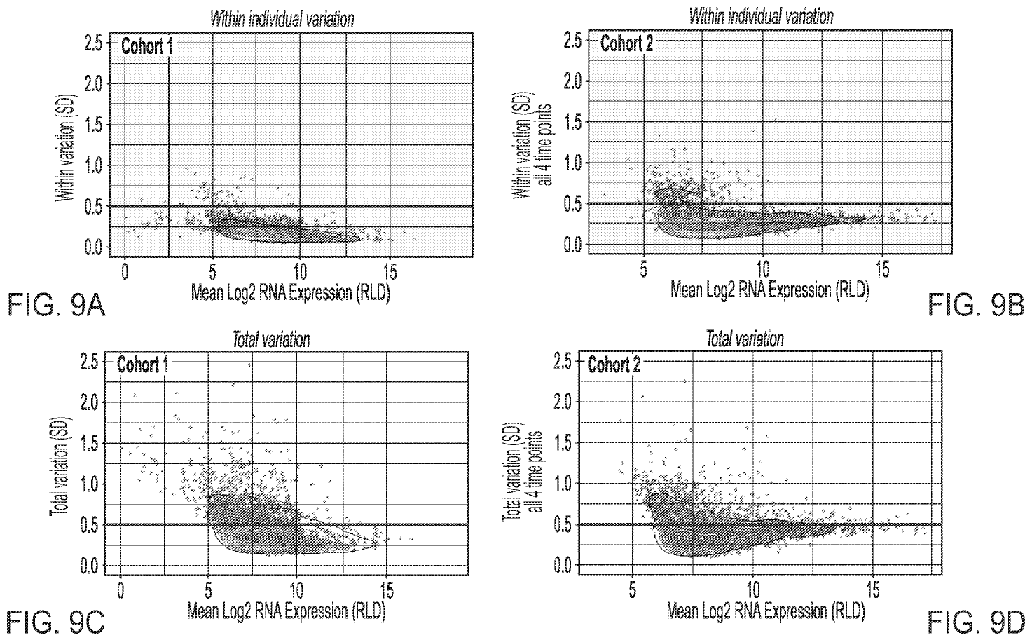

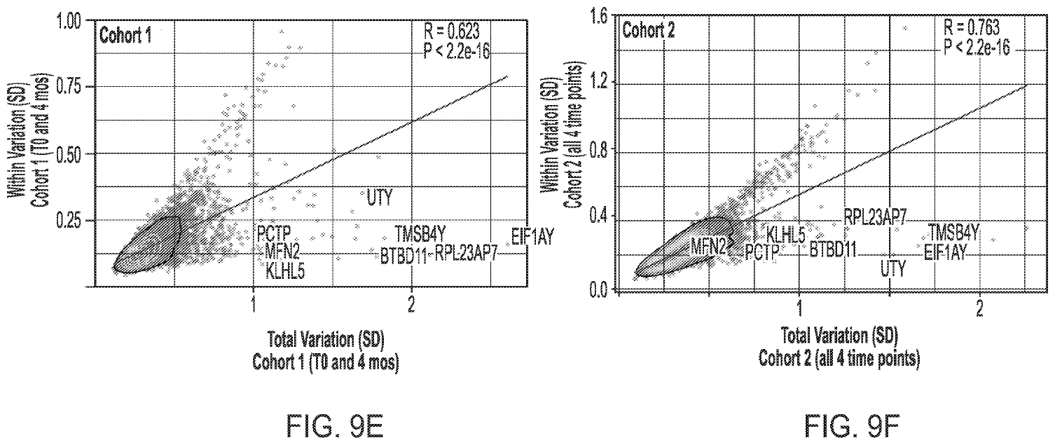

Within and between individual transcript variation in platelets is reproducible across cohorts. Together, the data in FIG. 1 indicate similar patterns of gene expression variation between cohorts 1 and 2: the average within-individual correlations were similar (0.983±0.13 vs 0.987±0.12) for each cohort, as were the average between-individual correlations (0.958±0.021 vs 0.947±0.024). The specific transcripts in each cohort that displayed the least and most overall (total) variation compared to within individual variation were further defined. As depicted in FIG. 2A and FIG. 9A-B, high within individual variation was limited to a small number of moderately expressed transcripts that were consistently variable in both cohorts 1 and 2. Transcripts that varied the most within individuals in both cohorts were enriched for those within the inflammatory and defense response gene ontologies (GO; FIG. 2B) (Bryois J, et al. Genome Res. 2017; 27:545-552; and Whitney A R, et al. Proc Natl Acad Sci USA. 2003; 100:1896-901). As shown in FIG. 2C and FIG. 9C-D, transcripts with high total variation spanned a broader range of expression levels, yet the extent of variation was still consistent between cohorts 1 and 2. The transcripts with the highest total variation predominantly overlapped those that varied the most within-individuals (FIG. 9E-F) leading to an enrichment in the inflammatory and defense response GO (FIG. 2D). In addition to inflammatory transcripts, several genes were noted with high total variation that were previously associated with sex, race, or platelet eQTLs (Edelstein L C, et al. Nat Med. 2013; 19:1609-16; Simon L M, et al. Am J Hum Genet. 2016; 98:883-97; and Simon L M, et al. Blood. 2014; 123(16):e37-45). However, unlike the inflammatory transcripts, most of these did not demonstrate high within individual variation (see bottom right quadrants of FIG. 9E-F).

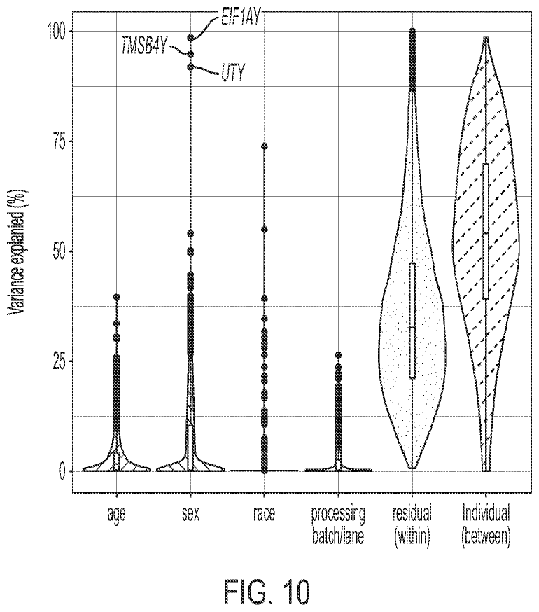

Variance partition analysis (Hoffman G E, Schadt E E. BMC Bioinformatics. 2016; 17:483) was used to further partition and quantify for each gene the amount of variation attributable to sex, race, and other covariates, and to decouple between from within and total variation. As shown in FIG. 10, for more than half of the genes, between individual variation was responsible for the majority (>50%) of gene expression variation, followed by residual within individual variation. On the other hand, sex and race affected a small number of genes. Other known covariates including age and sample processing contributed to a minor portion of variation for each gene.

Repeatability Defines Heritable Platelet Gene Expression and Predicts eQTL Genes.

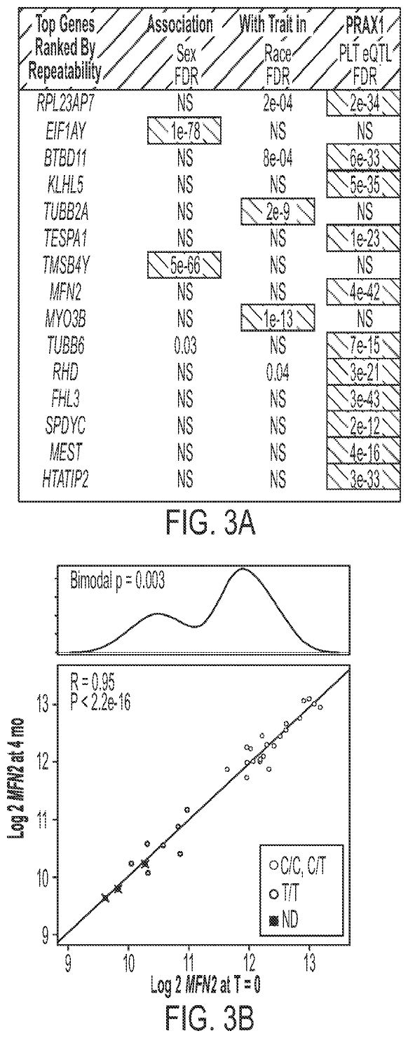

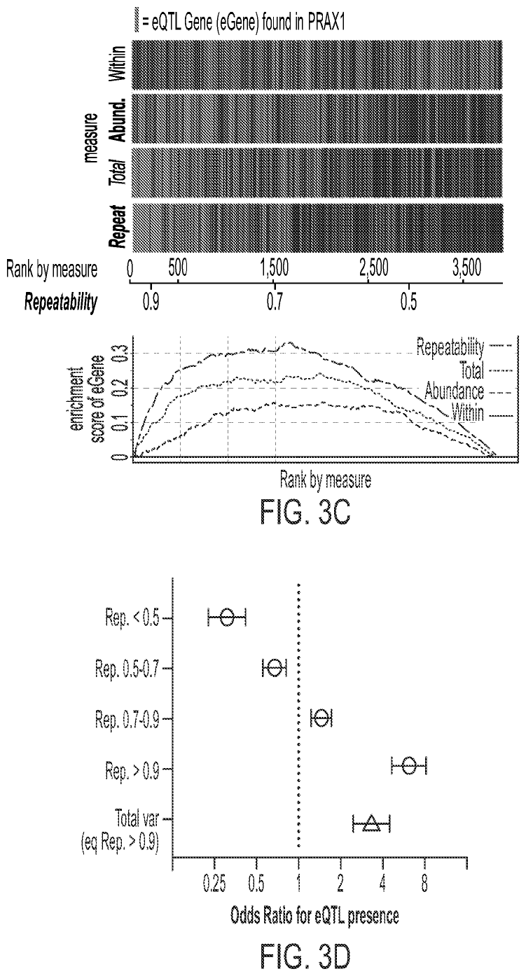

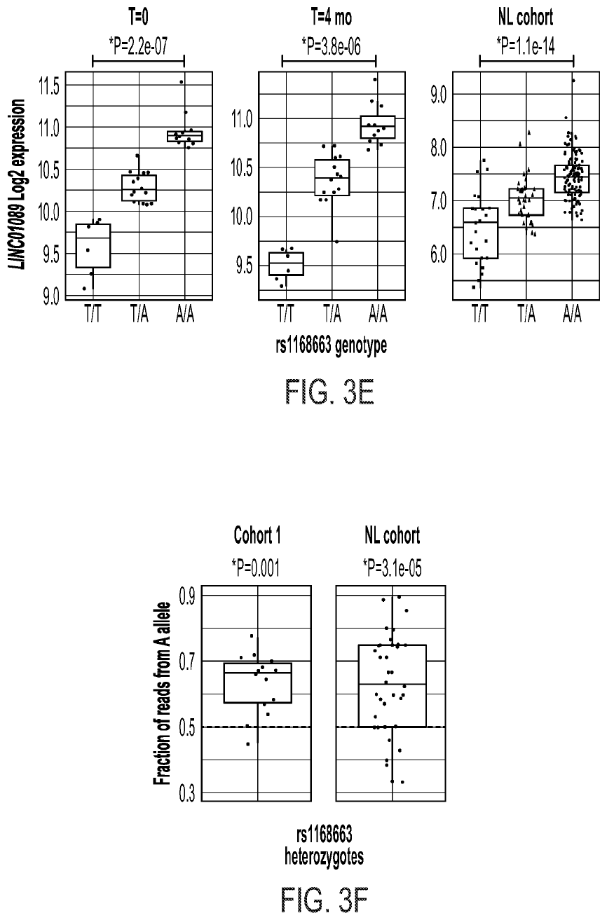

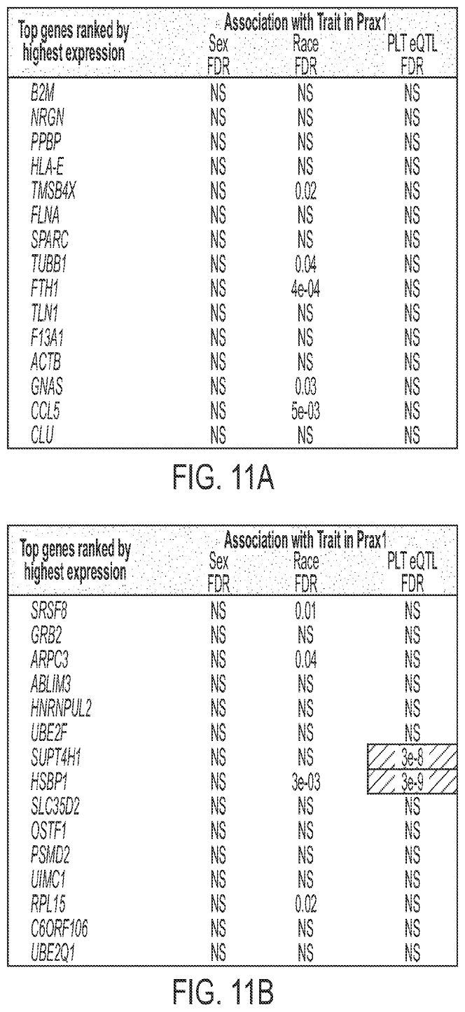

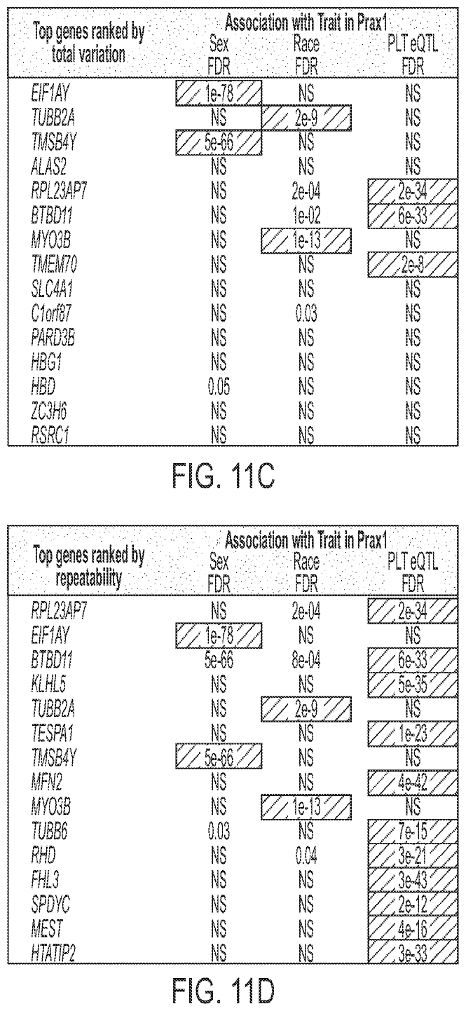

As discussed herein, it was tested whether repeatability, which captures the within and between individual variation in a single index (see methods), could be used to prioritize genes for eQTL analysis. Therefore, repeatability was calculated for each gene, and retrospectively tested whether repeatability is associated with genetic and heritable regulation of gene expression in platelets. To do this, the published PRAX1 (Simon L M, et al. Am J Hum Genet. 2016; 98:883-97) dataset was utilized as an independent (no overlap with the current cohorts) and cross-platform (microarray) validation dataset. PRAX1 previously associated platelet transcripts with sex and race, and identified 612 platelet eQTL genes. Cohort 1 was used for the analysis because it was larger in size than cohort 2 and better matched the diversity and demographics of PRAX1. The genes in the PRAX1 microarray were ranked according to the repeatability calculated by RNA-seq in cohort 1. Ranking by repeatability resulted in a significant enrichment for genes associated with sex, race, or eQTLs (p<2e-16), that was significantly greater than ranking genes by abundance, within-individual, or total variation (p<2e-6; also see FIG. 11). As shown in FIG. 3A, 100% of the top 15 repeatability ranked genes are significantly associated in expression with sex, race, or a cis-eQTL. As an example, MFN2 is an established platelet eQTL gene (Simon L M, et al. Am J Hum Genet. 2016; 98:883-97) that was among the most repeatable genes (repeatability=0.98). This is because MFN2 expression varies according to eQTL genotype (Simon L M, et al. Am J Hum Genet. 2016; 98:883-97) by more than 8 fold between individuals, while remaining relatively constant within individuals over time (FIG. 3B).

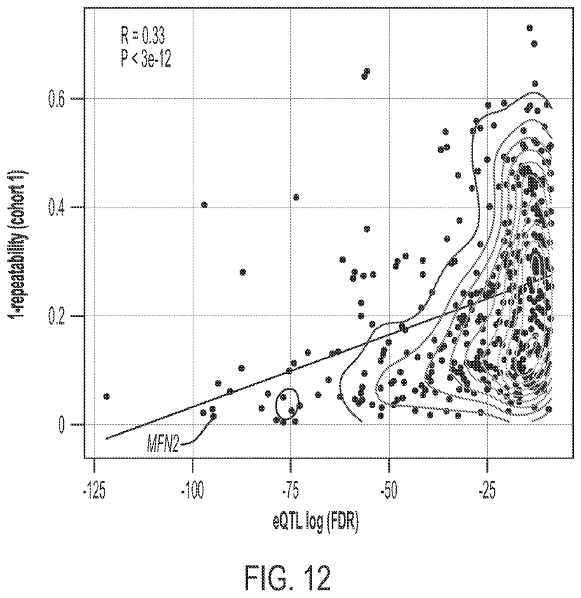

When assessing specifically the enrichment for eQTL genes, GSEA indicated a significant enrichment (p=0) of known eQTLs as repeatability increased, with those with repeatability >0.68 (leading edge) accounting for the enrichment (FIG. 3C). Significant enrichment was also observed when ranking by mean expression abundance or total variation, although the enrichment score for these measures was lower than for repeatability. According to binned analysis of odds ratios, the odds of identifying an eQTL for a gene with a repeatability <0.5 is 3.2 fold lower (p=5e-15) than testing a gene at random (FIG. 3D). The odds of identifying an eQTL for a gene with a repeatability >0.9 is 6.2 fold higher (p=6e-45) than random and 1.8 fold higher (p=0.006, adjusted) than ranking according to total variation. Furthermore, for known platelet cis-eQTLs, there was a significant correlation between the eQTL FDR and the repeatability of its associated transcript (FIG. 12). Thus, repeatability indicates an enrichment for and strength of cis-eQTL signal in platelets, and may be a useful filtering and prioritization strategy to identify genes with an eQTL signal.

Microarrays differ from RNA-seq in accuracy, sensitivity, and comprehensiveness, and some eQTL genes identifiable by RNA-seq may have been missed by PRAX1. Therefore, RNA-seq data was used to re-interrogate 238 genes with high repeatability (>0.9), yet with no cis-eQTLs previously found. These were tested for cis-eQTLs using a publicly available dataset (Simon L M, et al. Am J Hum Genet. 2016; 98:883-97; and Simon L M, et al. Blood. 2014; 123(16):e37-45) collected in the Netherlands, of platelet RNA-seq from 234 healthy individuals (NL cohort). Genetic variants were called from RNA-seq reads across each gene, and tested for association with RNA-seq abundance. Despite the known limitations of RNA-seq in calling genetic variants (e.g. most variants are located in promoters and introns), 11 new probable platelet eQTL genes were identified. In contrast, when analyzing the same number of genes with the lowest repeatability, no additional eQTLs were identified—a difference which was statistically significant (11/238 vs 0/238, p=0.0009, Fisher Exact). Allele Specific Expression (ASE) analysis of allelic imbalance, which measures the ratio of read counts coming from each allele within heterozygotes, confirmed a significant and directionally consistent within-sample eQTL effect on allelic imbalance for 8 of the 11 genes. An example of one of the novel platelet eQTL genes is long non-coding RNA LINC01089—one of the most repeatable (0.95) and abundant (top 10% by RNA-seq) transcripts in platelets. As shown in FIG. 3E, there is a strong additive allele dosage effect of rs1168663 on LINC01089 expression among cohort 1 individuals at both time points, and among individuals in the NL cohort. As shown in FIG. 3F, LINC01089 expression demonstrates significant allelic imbalance in cohort 1 and in the NL cohort. Together these data define several novel platelet cis-eQTLs, demonstrating the utility of repeatability from longitudinal expression data to predict heritable gene expression variation and prioritize targets for prospective identification of cis-eQTL genes.

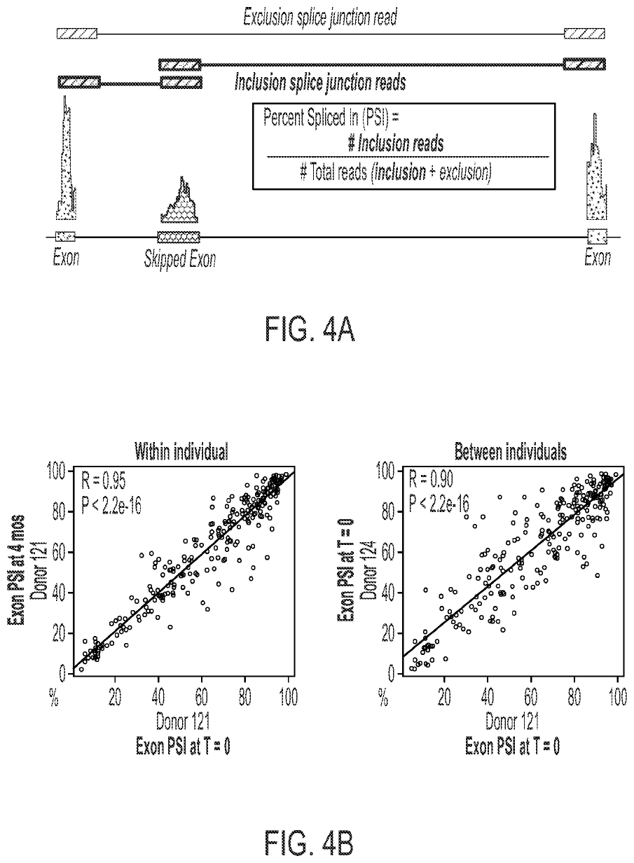

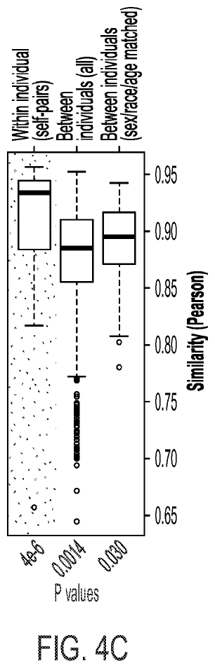

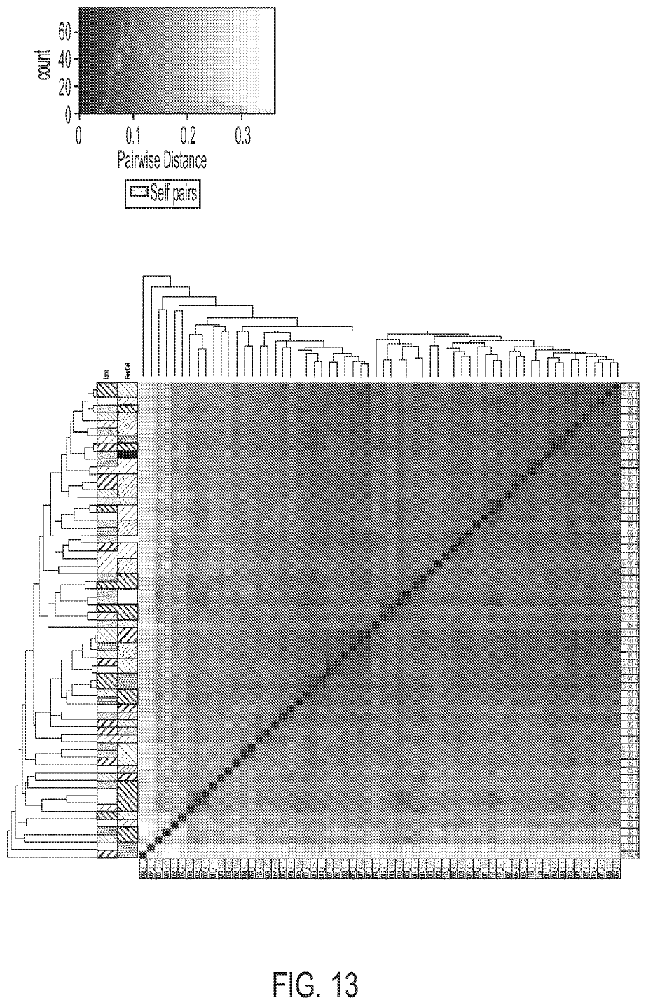

Alternative exon skipping in platelets is maintained within-individuals over time. To assess within- versus between-individual stability of alternative splicing, an alternative splicing event that is readily measured in RNA-seq data: exon skipping was the focus. Exon skipping events in cohort 1 (see methods) were stringently identified, and Percent of exon Spliced In (PSI; FIG. 4A) for each was calculated. As shown in FIG. 4B, there was a broad range of exon skipping levels among the different exon skipping events that was mostly consistent between individuals and within-individuals over time. Unsupervised clustering analysis using PSI resulted in a preference for within-individual clustering compared to between-individual clustering (FIG. 13), although this was not as robust as clustering based on expression. Nonetheless, the within-individual correlation of PSI was significantly higher than between-individual correlation, independent of age, race, or sex (FIG. 4B-C), suggesting a heritable component of exon skipping levels in platelets.

Identification of a platelet splice QTL associated with race that affects exon 14 skipping in SELP. Unlike eQTLs, platelet sQTLs have not previously been identified. Therefore, repeatability was used to prioritize exon skipping events, with the goal of identifying novel, robust, and physiologically relevant platelet cis-sQTLs. To this end, the within/between individual variation of PSI was assessed for each exon skipping event, and ranked each by repeatability. As shown in FIG. 5A, Exon 14 of SELL), which codes the leukocyte adhesion and platelet activation marker P-selectin, ranked second among repeatable exon skipping events. The difference in exon 14 exon skipping between donors, but stability within individuals is illustrated by the alignment plots in FIG. 5B and the correlation plot in FIG. 5C.

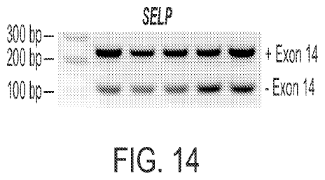

Exon 14 skipping predicts an in-frame deletion of the transmembrane domain of P-selectin. An exon 14 deficient isoform of P-selectin was previously detected in endothelial cells and platelets (Johnston G I, et al. J Biol Chem. 1990; 265:21381-5; McEver R P. Blood Cells. 1990; 16:73-80; and Ishiwata N, et. J Biol Chem. 1994; 269:23708-15) and at significant levels in the human circulation (McEver R P. Blood Cells. 1990; 16:73-80; Ishiwata N, et al. J Biol Chem. 1994; 269:23708-15; and Semenov A V, et al. Biochem Biokhimiia. 1999; 64:1326-35). PCR (FIG. 14), cloning, and Sanger sequencing verified that the RNA isoform predicted by RNA-seq in the cohorts matches the previously described soluble protein isoform in plasma. Together, this implicates exon 14 skipping as a heritable source of variability in P-selectin protein cell surface and soluble plasma levels between individuals.

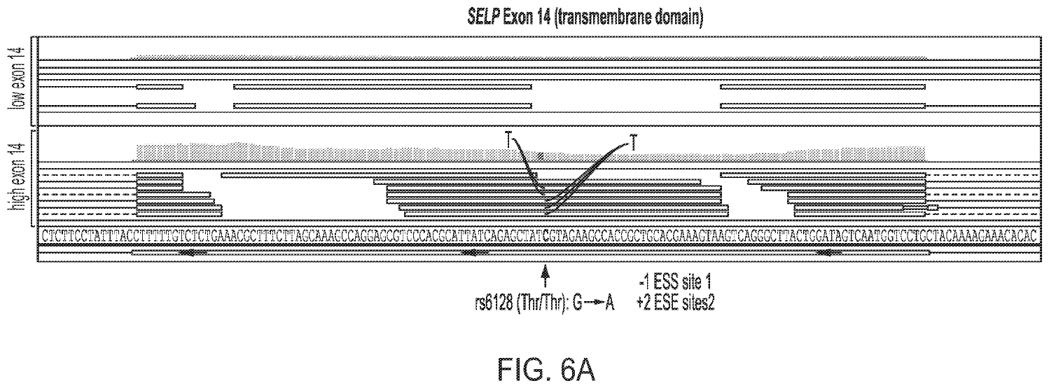

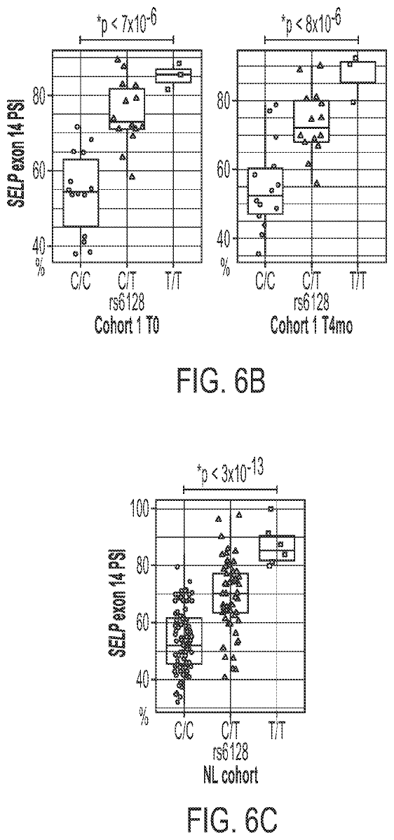

Previous studies have associated soluble P-selectin in the plasma with a variety of clinical and genetic factors including race and single nucleotide polymorphisms (SNPs) (Ataga K I, et al. N Engl J Med. 2017; 376:429-439; Lee D S, et al. J Thromb Haemost. 2007; 6:20-31; Burger P C, Wagner D D. Blood. 2003; 101:2661-2666; and Penman A, Hoadley S, et al. Am J Ophthalmol. 2015; 159:1152-1160.e2). As shown in FIG. 5D, a significant increase of SELL) exon 14 inclusion was observed among blacks/African Americans compared to whites at both time points. A search for the most likely responsible genetic variants identified a SNP, rs6128, within exon 14 of SELL) with a homozygous MAF (T/T) that is much higher among Africans (0.29) compared to Europeans (0.04) (Gibbs R A, et al. Nature. 2015; 526:68-74). Intriguingly, rs6128 has been associated with plasma P-selectin (Sun B B, et. al. Nature. 2018; 558:73-79) levels and diabetic retinopathy, especially among African Americans (Penman A, et al. Am J Ophthalmol. 2015; 159:1152-1160.e2). However, the direct relationship of rs6128 to soluble P-selectin is unclear since the C to T transition does not change the protein sequence or modify canonical splice sites. Bioinformatic analysis (Raponi M, et al. Hum Mutat. 2011; 32:436-444) of the sequence surrounding rs6128 predicted a net loss of an exonic splicing silencer (ESS) motif and a net gain of 2 exonic splicing enhancer motifs (ESE) (FIG. 6A). To determine whether rs6128 is associated with SELP exon 14 splicing in platelets, rs6128 genotypes was inferred from RNA-seq reads in cohort 1 and the NL cohort (Best M G, et. al. Cancer Cell. 2017; 32:238-252). As shown in FIG. 6B-C, the rs6128 SNP is significantly associated in both cohorts with SELP exon 14 skipping in platelets. The association of rs6128 with SELP exon 14 skipping was independent of age, sex, race, or population structure.

It was then tested whether the difference in rs6128 MAF between Africans and Europeans might account for the association of exon 14 skipping with race indicated in FIG. 5D. Consistent with this, there was no difference in exon 14 skipping levels between blacks/African Americans and whites after correcting for rs6128 (p=0.4 and 0.3 for T=0 and T=4 months respectively).

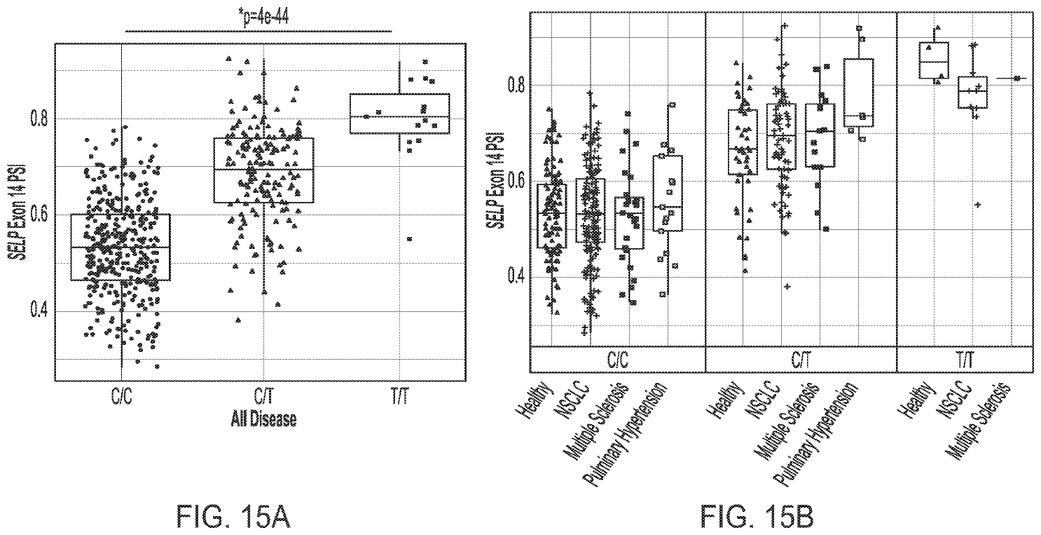

Given the importance of P-selectin in disease, the SELP exon 14 splicing analysis was extended to additional diseases also available through Best et. al. (Best M G, et. al. Cancer Cell. 2015; 28:666-676; and Best M G, et. al. Cancer Cell. 2017; 32:238-252). As shown in FIG. 15, none of the diseases examined (non-small cell lung cancer, multiple sclerosis, or pulmonary hypertension) was significantly associated with SELP exon 14 splicing, or the effect of rs6128 on splicing. This indicates that the levels of exon 14 skipping are stable within the individual, even in the context of environmental stressors which have been shown to trigger changes in platelet transcript abundance (Best M G, et al. Cancer Res. 2018; 78:3407-3412; and Best M G, et. al. Cancer Cell. 2017; 32:238-252).

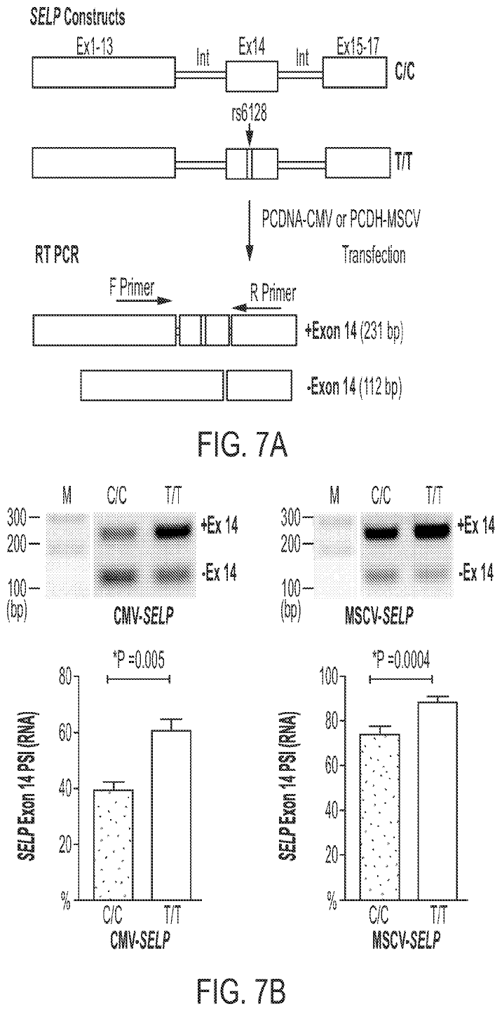

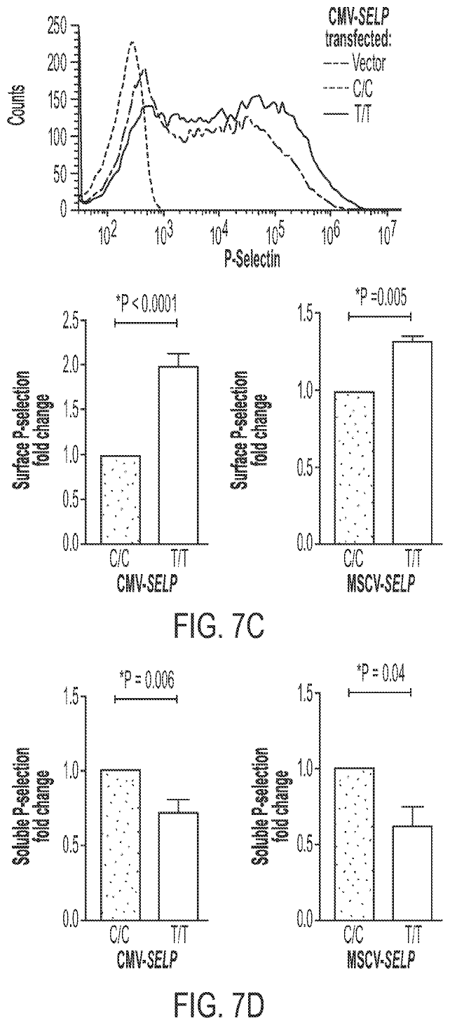

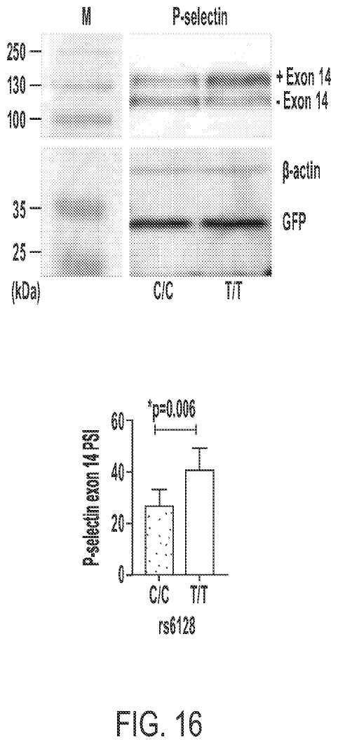

Rs6128 directly affects SELP exon 14 skipping and the proportion of soluble to surface P-selectin in vitro. Non-causal markers are commonly falsely identified in genetic association studies because of linkage with other unobserved variables (Platt A, et al. Genetics. 2010; 186:1045-52). To specifically test a causative effect of rs6128 on SELP exon 14 splicing, mini-gene constructs of the SELP ORF with rs6128 C/C or T/T were generated, and included introns flanking exon 14 (FIG. 7A). Since it is known that promoter differences can influence splicing (Cramer P, et al. Proc Natl Acad Sci USA. 1997; 94:11456-60), two different promoters (CMV or MSCV) were tested for each minigene construct. Constructs were expressed in HEK 293 cells, which lack endogenous P-selectin. Following transfection, RT-PCR analysis confirmed that the single nucleotide change from C/C to T/T resulted in a significant shift in the ratio of SELP RNA isoforms (FIG. 7B), in the direction consistent with RNA-seq results. This occurred for both promoters, but with a more pronounced shift observed for the CMV promoter. Western blot analysis indicated that the T/T variant resulted in a shift toward exon 14 inclusion in P-selectin protein (FIG. 16). As shown in FIG. 7C, and consistent with inclusion of the transmembrane domain, the single nucleotide change from C/C to T/T significantly increased (2 fold) the amount of surface P-selectin on HEK 293 cells. In contrast, the T/T variant significantly decreased the amount of soluble P-selectin in the supernatant as measured by ELISA (FIG. 7D). Together this data demonstrates a causal relationship between rs6128 genotype, the amount of exon 14 inclusion in SELP RNA, and the proportion of soluble to surface P-selectin expression.

Discussion Analysis of within-individual versus between individual variation in two independent cohorts indicated that the human platelet transcriptome is highly stable in healthy individuals for up to 4 years. There are few longitudinal studies available on primary nucleated cells for comparison. One study with similar design by Radich et. al. (Radich J P, et al. Genomics. 2004; 83:980-988) observed a 30% within-individual misclassification rate for leukocyte transcriptomes even after selecting for a gene signature that maximized variation between individuals. Although differences exist between this published study and results described herein, it was identified that platelets had a lower within-individual misclassification rate (10-12%) without signature selection. It is thought that their anucleate nature and 7-10 day lifespan moderate in vitro and in vivo RNA changes in platelets, promoting a stable and defined in vivo healthy gene expression signature.

Platelet gene expression profiling via RNA-sequencing is emerging as a relevant tool for platelet function studies, for defining the consequences and causes of disease, and for disease diagnostics (Best M G, et. al. Cancer Cell. 2015; 28:666-676; Kondkar A A, et al. J Thromb Haemost. 2010; 8:369-78; Edelstein L C, et al. Nat Med. 2013; 19:1609-16; Schubert S, et al. Blood. 2014; 124:493-502; Kong X, et al. Thromb Haemost. 2017; 117:962-970; Best M G, et. al. Cancer Cell. 2017; 32:238-252; and Campbell R A, et. al. Blood. 2019; blood-2018-09-873984). However, most published studies to date, have relied on single-time point comparisons of the platelet transcriptome between a disease cohort and healthy subjects. As healthy subjects are often used as the “baseline” or “control” condition in these studies, understanding whether the platelet transcriptome is durable—or not—in health is important to understanding the robustness of these comparisons. The finding that the platelet transcriptome is generally stable over 4 years in healthy individuals lends validity to these comparisons. In depth analysis of the individual transcripts that do vary within and between individuals also suggest some limitations and caveats.

Although most transcripts were stable, a few transcripts varied substantially within individuals. Most within-variable transcripts were related to inflammation. These may inform studies evaluating the effects of inflammation on platelet gene expression, and may be of relevance to clinical findings that inflammatory stress is associated with platelet counts and function⁵⁸. The range of variation for inflammatory transcripts in health compared to overt inflammatory disease may be worth investigating when assessing the impact of inflammatory gene changes on disease.

Major platelet expression differences were observed between individuals. Sex and race accounted for some major differences. Other sources of individual variation contributed more. The data suggest a prominent role for cis-eQTLs. Regardless of the source of variation, the propensity of a gene to vary between (or within) healthy individuals might be taken into consideration when interpreting differential disease-gene studies and designing experiments for validation. Genes with high inherent variation require larger sample sizes to reach statistical confidence. When sample sizes are small, false positives are more likely for genes with high inherent variation. Correction for known covariates might be helpful in this regard. While corrections for sex, race, and age are often considered in differential gene expression analyses, eQTLs are normally unavailable or ignored.

Stability information may also guide (along with the Minimum Information for Publication of Quantitative Real-Time PCR Experiments (MIQE (Bustin S A, et al. Clin Chem. 2009; 55:611-622) guidelines) the selection of reference genes for normalization controls. Within/between stable genes such as SYK, AKT1/2, GP1BA, ACTB might be good choices. On the other hand, highly within-variable genes, such as the gene TUBB1, should generally be avoided as reference genes.

As an application of the longitudinal dataset, repeatability was used as a filtering strategy to identify new platelet eQTLs. Because of the limitation of multiple testing (Altshuler D, et al. Science. 2008; 322:881-888), even large scale gene expression association studies employ a filtering and prioritization strategy to circumvent testing thousands of genes (and even more alternative splice events) against millions of genetic loci. Common filtering strategies include hard filtering on abundance (Kumar V, et. al. PLoS Genet. 2013; 9:e1003201) or total variance. However, filtering for total variance alone will also enrich for transcripts overly-influenced by technical or environmental noise. To avoid this, several studies (Barendse W. BMC Genomics. 2011; 12:232; Carlborg O, et al. Bioinformatics. 2004; 21:2383-93; and Hoffman G E, Schadt E E. BMC Bioinformatics. 2016; 17:483) have suggested using repeatability instead. Here, platelet longitudinal data was used to experimentally test this idea. A significant enrichment for cis-eQTLs was observed among the most repeatable genes that significantly improved the ability to identify eQTL genes compared to using abundance or total variation.

Although significantly enriched for eQTLs, the association with repeatability was not perfect. Cohort 1 was assayed on a different platform (RNA-seq vs microarray), was smaller (31 vs 154), and had similar, but not the same demographics to PRAX1 (race (Blacks/African Americans): 45% vs 42%, ns; sex (M): 49% vs 32%, ns; age: 42+/−11 vs 29+/−7, p<0.05). Repeatability measured in the same samples used for eQTL analysis would presumably result in the best predictions. However, large longitudinal studies are often impractical because of the costs and challenges of repeated sampling. Moreover, repeatability can add a layer of confidence to eQTL results if measured in an independent cohort. For this, larger sample sizes that reflect the demographics and environment of the test cohorts will presumably fare better. Cohorts that are too small to capture genetic variation (i.e. low MAF eQTLs), or are subject to systematic environmental perturbations, will suffer from overall lower repeatability, and lack sensitivity to predict eQTLs. Additional studies are needed to determine how repeatability might be applied more generally in the analysis of additional datasets, cell types, and to handle gene-environment interactions.

An additional RNA-seq cohort (NL cohort) was used to test for unreported platelet eQTL genes among the most repeatable genes in cohort 1. Of the identified eQTLs, 22/27 (for 7/11 eQTL genes) have been reported in other tissues (The Genotype-Tissue Expression (GTEx) Project). Noteworthy among eQTL candidate genes is TECPR2, which was previously associated with platelet counts by GWAS (Astle W J, et. al. Cell. 2016; 167:1415-1429.e19). Long non-coding RNA eQTL genes were also identified: LINC01089, MAGI2-AS3, KANSL1-AS1. Like these 3 genes, it was found that non-coding RNAs are generally stable within individuals, yet more variable between individuals compared to protein-coding RNAs, suggesting more genetic diversity among non-coding RNAs. Long non-coding RNAs have gained attention for their multi-faceted ability to regulate gene expression, but are understudied in platelets.

Repeatability was further applied to prioritize exon skipping events to find those with the greatest likelihood of identifying a biologically tractable sQTL signal. A robust association between rs6128 and SELP exon 14 skipping was identified. A significant association between rs6128 splicing and exon 14 skipping was also identified in whole blood samples (Zhernakova D V, et. al. Nat Genet. 2017; 49:139-145), further strengthening the findings.

To establish causality, we performed transfection experiments in HEK 293 cells, which advantageously do not express endogenous SELP. The results strongly implicate rs6128 as causal for differential SELP exon 14 splicing in platelets, but do not rule out potential contributing effects of the endogenous promoter, or of additional associated or linked variants. The confirmation of a platelet observation in an unrelated cell line suggests rs6128 may affect SELP splicing in multiple tissues such as endothelial cells—another major producer of P-selectin.

Surface P-selectin mediates leukocyte interactions and inflammation, is involved in atherogenesis, and plays a role in tumor metastasis. Soluble P-selectin is a functionally and clinically relevant platelet protein in the circulation associated with a variety of diseases (Ataga K I, et. al. N Engl J Med. 2017; 376:429-439; and Ludwig R J, et al. Expert Opin Ther Targets. 2007; 11:1103-1117). While a major source of soluble P-selectin is related to activation induced shedding, a significant amount is heritable. Associations between rs6128 and soluble P-selectin protein levels in plasma have been reported (Penman A, et al. Am J Ophthalmol. 2015; 159:1152-1160.e2; and Sun B B, Maranville J C, Peters J E, et. al. Genomic atlas of the human plasma proteome. Nature. 2018; 558:73-79). The observed effects of rs6128 on exon 14 SELP splicing and soluble versus surface P-selectin localization establishes a link between these observations. They may explain previous clinical studies that have associated rs6128 with plasma P-selectin and diabetic retinopathy in African Americans (Penman A, et al. Am J Ophthalmol. 2015; 159:1152-1160.e2). Finally, the finding that SELP is differentially spliced according to race may be therapeutically relevant in light of promising clinical trials that effectively used P-selectin blockade to treat pain crisis in Sickle Cell Anemia, a disease that predominantly affects individuals of African descent (Ataga K I, et. al. N Engl J Med. 2017; 376:429-439).

While human platelets have a rich repertoire of RNAs, platelet RNA expression differs between individuals in health and disease, and is associated with platelet function; and single time-point studies of the platelet transcriptome are increasingly utilized for biological discovery in human health and disease, the results disclosed herein provides new information.

The results disclosed show that platelet RNA expression is stable and repeatable for up to 4 years when assessed over time in healthy human donors; the integrated use of longitudinal repeatability metrics significantly enhances the discovery of genetic variants that affect gene expression and splicing; and genetic variant in the SELP gene directs the removal of the P-selectin transmembrane domain.

The results disclosed show that although anucleate, platelets possess a rich and dynamic transcriptome. Platelet transcriptomics are increasingly used, with applications ranging from cancer diagnostics to gene discovery. The results disclosed herein adds to the field by establishing, the stability—or reproducibility—of platelet gene expression and splicing in healthy donors assessed repeatedly for up to four years. This type of longitudinal assessment has been lacking for any primary human cell, let alone platelets. The results show that the platelet transcriptome is exquisitely stable in health, which may aid comparisons in disease settings, and enhance diagnostics and prognostics that use platelet RNA. Moreover, the data show that integrating measures of repeatability (e.g. between versus within-individual variation of platelet RNA expression and splicing) results in improved detection of genes affected by nearby genetic variants. This technique can be applied in the discovery of a platelet SELP sQTL. Functionally, this splice QTL directs the removal of the transmembrane domain of P-selectin. The findings disclosed herein show that this transmembrane domain deletion is reduced in blacks compared to whites. In vitro, this increases surface, but decreases soluble, P-selectin, suggesting that this may have implications in diseases more common in blacks where P-selectin is a therapeutic target (e.g., sickle cell disease).

1. A method for making a transcriptome-wide expression profile of a biological sample, said method comprising:
a) measuring the expression levels of one or more genes present in a first biological sample, wherein said measuring comprises RNA sequencing;
b) determining the expression levels of the genes from the measured expression levels obtained in step a);
c) combining the results of steps a) and b) to produce a transcriptome-wide expression profile; and
d) providing the transcriptome-wide expression profile as a data set, wherein the first biological sample consists of isolated platelets.

2. The method of claim 1, wherein the method is repeated at least once.

3. The method of claim 1, wherein the method is repeated on a second biological sample, wherein the second biological sample consists of isolated platelets.

4. The method of claim 3, wherein the first and second biological samples are obtained from the same subject or from different subjects.

5. The method of claim 4, wherein the first and second biological samples are obtained from the same subject at a first time point and a second time point.

6. The method of claim 5, wherein the first and second time points are different time points.

7. The method of claim 6, wherein the transcriptome-wide expression profile from the first time point is compared to the transcriptome-wide expression profile from the second time point.

8. The method of claim 1, wherein said method further comprises repeating the steps thereof until a validated transcriptome-wide expression profile is identified.

9. The method according to claim 1, wherein the expression levels are measured on a device selected from the group consisting of a microarray, a bead array, a liquid array, and a nucleic-acid sequence.

10. The method of claim 4, wherein steps a)-d) are repeated on each biological sample.

11. The method of claim 10, wherein the transcriptome-wide expression profile of the subjects is compared.

12. The method of claim 5, wherein the first and second biological samples are obtained from different subjects, further comprising obtaining additional biological samples from the subjects, wherein the additional biological samples are obtained at different time points.

13. The method of claim 12, wherein the transcriptome-wide expression profiles from the first time point is compared to the transcriptome-wide expression profiles from the second time point.

14. A method of identifying gene expression differences between two transcriptome-wide expression profiles, the method comprising: determining one or more variations in subject's platelet transcriptome using the method of claim 1, wherein the method is performed at different time points, thereby identifying a gene expression difference.

15. A method of measuring time dependent gene expression differences in platelets from a subject, the method comprising determining one or more variations in subject's platelet transcriptome using the method of claim 1, wherein the method is performed at different time points, thereby identifying a gene expression difference; and comparing said time-dependent changes from the subject to a reference.


SEQUENCE LISTING (2 sequences)

SEQ ID NO: 1 - DNA, 20 bp, Artificial Sequence
  Synthetic construct
gtcaactaccgtgccaacct

SEQ ID NO: 2 - DNA, 20 bp, Artificial Sequence
  Synthetic construct
taaggactcgggtcaaatgc